(19) United States
(12) Patent Application Publication
(10) Pub. No.: US 2019/0010167 A1
(43) Pub. Date: Jan. 10, 2019

(54) INHIBITORS OF THE MENIN-MLL INTERACTION

(71) Applicant: Vitae Pharmaceuticals, Inc., Madison, NJ (US)

(72) Inventors: David A. Claremon, Maple Glen, PA (US); Chenggou Dong, Staten Island, NY (US); Yi Fan, Doylestown, PA (US); Lanqi Jia, Horsham, PA (US); Suresh B. Singh, Kendall Park, NJ (US); Shankar Venkatraman, Lansdale, PA (US); Jing Yuan, Lansdale, PA (US); Yajun Zheng, Hockessin, DE (US); Linghang Zhuang, Chalfont, PA (US)

(21) Appl. No.: 16/064,998

(22) PCT Filed: Dec. 21, 2016
(86) PCT No.: PCT/US2016/068016

§ 371 Date: Jun. 21, 2018

Related U.S. Application Data
(60) Provisional application No. 62/270,973, filed on Dec. 22, 2015.

(51) Int. Cl. C07D 495/04; C07D 513/04; C07D 471/10; C07D 487/10

(52) U.S. Cl. CPC C07D 495/04; C07D 487/10; C07D 471/10; C07D 513/04

(57) ABSTRACT

The present invention is directed to inhibitors of the interaction of menin with MLL and MLL fusion proteins, pharmaceutical compositions containing the same, and their use in the treatment of cancer and other diseases mediated by the menin-MLL interaction.

### TECHNICAL FIELD

The present invention is directed to inhibitors of the interaction of menin with MLL and MLL fusion proteins, pharmaceutical compositions containing the same, and their use in the treatment of cancer and other diseases mediated by the menin-MLL interaction.

### BACKGROUND

The mixed-lineage leukemia (MLL) protein is a histone methyltransferase that is mutated in clinically and biologically distinctive subsets of acute leukemia. Rearranged mixed lineage leukemia (MLL-r) involves recurrent translocations of the 11q23 chromosome locus which lead to an aggressive form of acute leukemia with limited therapeutic options. These translocations target the MLL gene creating an oncogenic fusion protein comprising the amino-terminus of MLL fused in frame with more than 60 different fusion protein partners. Menin, a ubiquitously expressed, nuclear protein encoded by the multiple endocrine neoplasia type 1 (MEN1) tumor suppressor gene, has a high affinity binding interaction with MLL fusion proteins and is an essential co-factor of oncogenic MLL-r fusion proteins (Yokoyama et al., 2005, Cell, 123:207-18; Cierpicki & Grembecka, 2014, Future Med. Chem., 6:447-462). Disruption of this interaction leads to selective growth inhibition and apoptosis of MLL-r leukemia cells both in vitro (Grembecka et al., 2012, Nat. Chem. Biol., 8:277-284) and in vivo (Yokoyama et al., 2005, op. cit.; Borkin et al., 2015, Cancer Cell, 27:589-602).

The menin-MLL complex plays a role in castration-resistant/advanced prostate cancer, and a menin-MLL inhibitor has been shown to reduce tumor growth in vivo (Malik et al., 2015, Nat. Med., 21:344-352). Additionally, a menin-MLL inhibitor has been shown to enhance human β cell proliferation (Chamberlain et al., 2014, J. Clin. Invest., 124:4093-4101), supporting a role for inhibitors of the menin-MLL interaction in the treatment of diabetes (Yang et al., 2010, Proc Natl Acad Sci USA., 107:20358-20363). The interaction between menin and MLL or MLL fusion proteins is an attractive target for therapeutic intervention, and there is a need for novel agents that inhibit the menin-MLL interaction for the treatment of various diseases and conditions, including leukemia, other cancers and diabetes.

### SUMMARY

The present invention provides inhibitors of the menin-MLL interaction, such as a compound of Formula (I):

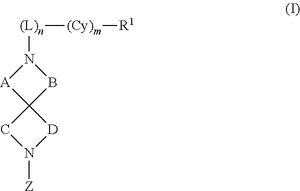

or a pharmaceutically acceptable salt thereof, wherein constituent variables are defined herein.

The present invention further provides a pharmaceutical composition comprising a compound of Formula (I), or a pharmaceutically acceptable salt thereof, and at least one pharmaceutically acceptable carrier.

The present invention further provides a method of inhibiting the interaction between menin and MLL comprising contacting the menin and MLL with a compound of any one of Formula (I), or a pharmaceutically acceptable salt thereof.

The present invention further provides a method of treating cancer in a patient comprising administering to the patient a therapeutically effective amount of a compound of Formula (I), or a pharmaceutically acceptable salt thereof.

The present invention further provides a method of treating insulin resistance, pre-diabetes, diabetes, risk of diabetes, or hyperglycemia in a patient comprising administering to the patient a therapeutically effective amount of a compound of Formula (I), or a pharmaceutically acceptable salt thereof.

### DETAILED DESCRIPTION

The present invention provides inhibitors of the menin-MLL interaction, such as a compound of Formula (I):

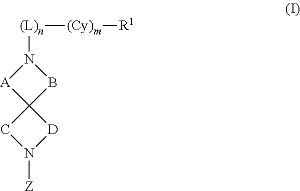

or a pharmaceutically acceptable salt thereof, wherein:

A, B, C, and D are each independently selected from —C(R^{A1})(R^{A2})—, —C(R^{A1})(R^{A2})—C(R^{A1})(R^{A2})—, —C(═O)— and —C(R^{A1})(R^{A2})—C(═O)—, wherein no more than one of A, B, C, and D is —C(R^{A1})(R^{A2})—C(═O)— or —C(═O)—;

L is selected from —C₁₋₆ alkylene- and —(C₁₋₄ alkylene)ₐ-Y—(C₁₋₄ alkylene)_{b}-, wherein the C₁₋₆ alkylene group and any C₁₋₄ alkylene group of the —(C₁₋₄ alkylene)ₐ-Y—(C₁₋₄ alkylene)_{b}- group is optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino;

Y is independently selected from —O—, —S—, —S(═O)—, —S(═O)₂—, —C(═O)—, —C(═O)NR^{y}—, —C(═O)O—, —OC(═O)NR^{y}—, —NR^{y}—, —NR^{y}C(═O)O—, —NR^{y}C(═O)NR^{y}—, —S(═O)₂NR^{y}—, —C(═NR^{z})—, and —C(═NR^{z})—NR^{y}—, wherein each R^{y} is independently selected from H or C₁₋₆ alkyl, and wherein each R^{z} is independently selected from H, C₁₋₆ alkyl, and CN;

Cy is C₆₋₁₄ aryl, C₃₋₁₈ cycloalkyl, 5-16 membered heteroaryl, or 4-18 membered heterocycloalkyl, each of which is optionally substituted with 1, 2, 3, or 4 substituents independently selected from R^{Cy};

each R^{Cy} is independently selected from halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R_{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R_{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R¹ is H, Cy¹, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2} and S(O)₂NR^{c2}R^{d2}, wherein said C₁₋₆ alkyl, C₂₋₆ alkenyl, and C₂₋₆ alkynyl are each optionally substituted by 1, 2, 3, or 4 substituents independently selected from CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

Z is a group of Formula (Z-1) or (Z-2):

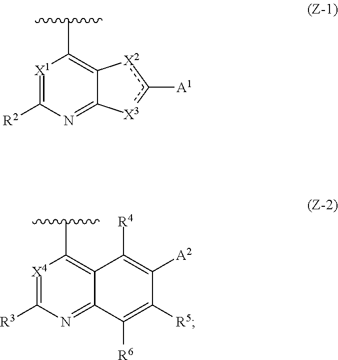

X¹ is selected from CR⁷ and N;

X² is selected from S, CR⁸, and N;

X³ is selected from CH, S, O, and NR^{N};

X⁴ is selected from CR⁹ and N;

[symbol] represents a single or double bond, wherein one [symbol] in the group of Formula (Z-1) is a double bond and the other is a single bond;

each R², R³, R⁴, R⁵, R⁶, R⁷, R⁸, and R⁹ is independently selected from H, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, and C₂₋₆ alkynyl are each optionally substituted by 1, 2, 3, or 4 substituents independently selected from CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

R^{N} is H or C₁₋₆ alkyl optionally substituted by 1, 2, 3, or 4 substituents independently selected from CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

A¹ is selected from H, halo, C₁₋₆ alkyl, C₁₋₄ haloalkyl, C₁₋₄ haloalkoxy, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, are each optionally substituted by 1, 2, 3, or 4 substituents independently selected from CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};

A² is selected from H, halo, C₁₋₆ alkyl, C₁₋₄ haloalkyl, C₁₋₄ haloalkoxy, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};

each R^{A1} is independently selected form H, halo, C₁₋₄ alkyl, C₁₋₄ alkoxy, C₁₋₄ haloalkyl, C₁₋₄ haloalkoxy, CN, NO₂, and OH;

each R^{A2} is independently selected form H, halo, C₁₋₄ alkyl, C₁₋₄ alkoxy, C₁₋₄ haloalkyl, C₁₋₄ haloalkoxy, CN, NO₂, and OH;

each Cy¹ is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy1};

each R^{Cy1} is independently selected from halo, C₁₋₆ alkyl, C₁₋₄ haloalkyl, C₁₋₄ cyanoalkyl, C₂₋₆ alkenyl, C₂₋₆ alkynyl, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, C(═NR^{e6})NR^{c6}R^{d6}, NR^{c6}C(═NR^{e6})NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}, wherein said C₁₋₆ alkyl, C₂₋₆ alkenyl, and C₂₋₆ alkynyl are each optionally substituted by 1, 2, 3, or 4 substituents independently selected from CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, C(═NR^{e6})NR^{c6}R^{d6}, NR^{c6}C(═NR^{e6})NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6};

each R^{a1}, R^{b1}, R^{c1}, R^{d1}, R^{a2}, R^{b2}, R^{c2}, R^{d2}, R^{a3}, R^{b3}, R^{c3}, R^{d3}, R^{a4}, R^{b4}, R^{c4}, R^{d4}, R^{a5}, R^{b5}, R^{c5}, R^{d5}, R^{a6}, R^{b6}, R^{c6}, and R^{d6} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycloalkyl)-C₁₋₆ alkyl, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl, C₃₋₁₀ cycloalky-C₁₋₆ alkyl, (5-10 membered heteroaryl)-C₁₋₆ alkyl, and (4-10 membered heterocycloalkyl)-C₁₋₆ alkyl are each optionally substituted with 1, 2, 3, 4, or 5 substituents independently selected from R^{g};

each R^{e1}, R^{e2}, R^{e3}, R^{e4}, R^{e5}, and R^{e6} is independently selected from H, C₁₋₄ alkyl, and CN;

each R^{g} is independently selected from the group consisting of OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO-C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, C₁₋₆ alkylcarbonyl, and C₁₋₆ alkoxycarbonyl;

n is 0 or 1;

m is 0 or 1;

a is 0 or 1; and

b is 0 or 1,

wherein any cycloalkyl or heterocycloalkyl group is optionally further substituted by 1 or 2 oxo groups; and

wherein the compound is not:

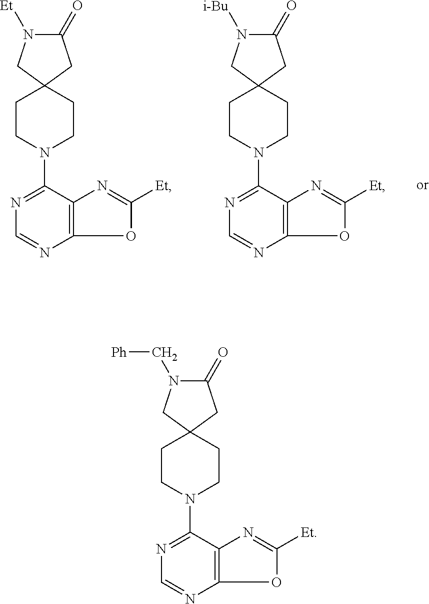

In some embodiments, A, B, C, and D are each independently selected from —C(R^{A1})(R^{A2})—, —C(R^{A1})(R^{A2})—C(R^{A1})(R^{A2})—, and —C(═O)—, wherein no more than one of A, B, C, and D is —C(═O)—.

In some embodiments, A, B, C, and D are each independently selected from —C(R^{A1})(R^{A2})— and —C(R^{A1})(R^{A2})—C(R^{A1})(R^{A2})—.

In some embodiments, the moiety formed by A, B, C, D, the nitrogen atom to which A and B are attached, the nitrogen atom to which C and D are attached, and the spirocyclic carbon atom, is selected from the following spirocycles having Formulae (i) to (x):

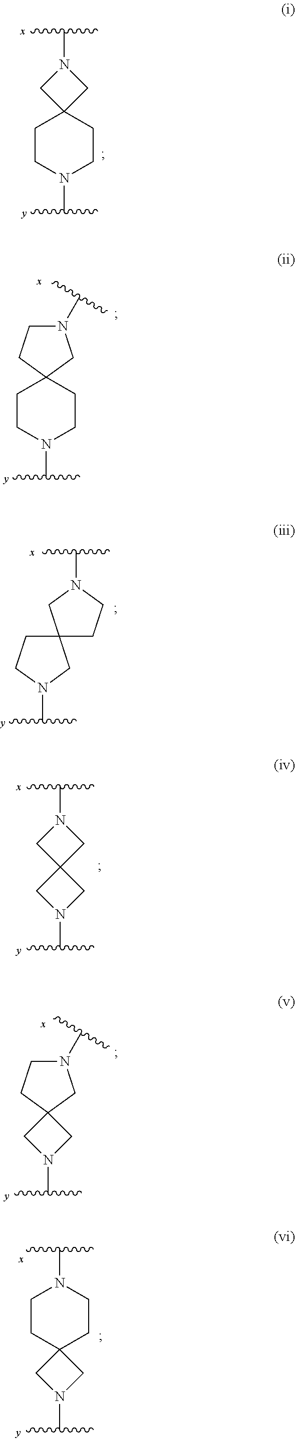

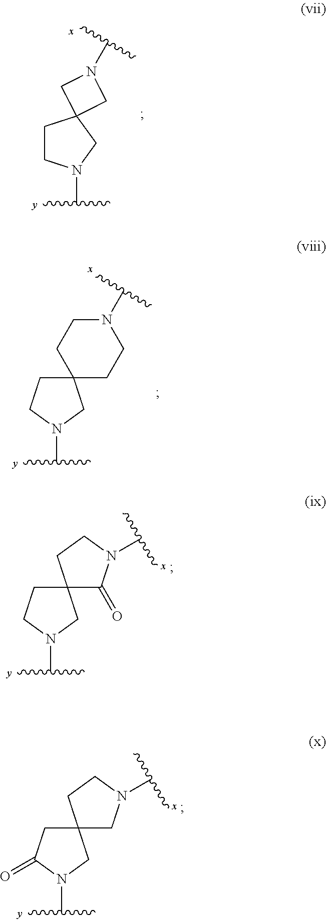 [SMILES: *N1CCC2(C1)CN(*)C2 ; *N1CCC2(CC1)CCN(*)C2 ; *N1CCC2(CCN(*)C2=O)C1 ; *N1CCC2(CC(=O)N(*)C2)C1]

wherein “x” indicates the point of attachment to L and “y” indicates the point of attachment to Z.

In some embodiments, L is —C₁₋₆ alkylene optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino.

In some embodiments, L is selected from —(C₁₋₄ alkylene)ₐ-Y—(C₁₋₄ alkylene)_{b}- wherein the any C₁₋₄ alkylene group of the —(C₁₋₄ alkylene)ₐ-Y—(C₁₋₄ alkylene)_{b}- group is optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino.

In some embodiments, L is selected from methylene, ethylene, and —Y—(C₁₋₄ alkylene)_{b}- wherein the methylene, ethylene, and C₁₋₄ alkylene group are each optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino.

In some embodiments, L is selected from methylene, ethylene, and —(C═O)—(C₁₋₄ alkylene)_{b}-, wherein the methylene, ethylene, and C₁₋₄ alkylene group are each optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino.

In some embodiments, n is 0.

In some embodiments, n is 1.

In some embodiments, Cy is phenyl, C₃₋₇ cycloalkyl, 5-10 membered heteroaryl, or 4-10 membered heterocycloalkyl, each of which is optionally substituted with 1, 2, or 3 substituents independently selected from R^{Cy} and wherein the cycloalkyl or heterocycloalkyl group is optionally further substituted by 1 or 2 oxo groups.

In some embodiments, Cy is selected from phenyl, tetrahydrofuranyl, tetrahydropyranyl, cyclohexyl, cyclopentyl, cyclobutyl, cyclopropyl, 4,5-dihydrothiazol-2-yl, indolyl, dihydrobenzo[d]oxazolyl, 1,3-dihydro-2H-benzo[d]imidazolyl, piperidinyl, pyrrolidinyl, oxetanyl, and tetrahydro-2H-thiopyran-1,1-dioxide-4-yl.

In some embodiments, Cy is selected from phenyl, tetrahydrofuran-2-yl, tetrahydrofuran-3-yl, tetrahydro-2H-pyran-2-yl, tetrahydro-2H-pyran-3-yl, tetrahydro-2H-pyran-4-yl, cyclohexyl, cyclopentyl, cyclobutyl, cyclopropyl, 4,5-dihydrothiazol-2-yl, indol-3-yl, indol-5-yl, indol-6-yl, dihydrobenzo[d]oxazol-6-yl, 1,3-dihydro-2H-benzo[d]imidazol-5-yl, piperidin-3-yl, piperidin-2-yl, pyrrolidin-2-yl, oxetan-3-yl, and tetrahydro-2H-thiopyran-1,1-dioxide-4-yl.

In some embodiments, R^{Cy} is independently selected from halo, C₁₋₆ alkyl, C₁₋₄ haloalkyl, C₆₋₁₀ aryl, C₃₋₁₀ cycloalkyl, 5-10 membered heteroaryl, 4-10 membered heterocycloalkyl, CN, NO₂, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, R^{c1}S(O)₂R^{b1}, S(O)₂R^{b1}, and S(O)₂NR^{c1}R^{d1}, wherein said C₁₋₆ alkyl, C₆₋₁₀ aryl, C₃₋₁₀ cycloalkyl, 5-10 membered heteroaryl, and 4-10 membered heterocycloalkyl are each optionally substituted by 1, 2, or 3 substituents independently selected from CN, NO₂, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1} and wherein the cycloalkyl or heterocycloalkyl group is optionally further substituted by 1 or 2 oxo groups.

In some embodiments, R^{Cy} is independently selected from halo, C₁₋₆ alkyl, C₁₋₄ haloalkyl, CN, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein said C₁₋₆ alkyl is optionally substituted by 1 or 2 substituents independently selected from CN, OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}.

In some embodiments, m is 0.

In some embodiments, m is 1.

In some embodiments, R¹ is independently selected from H, halo, C₁₋₆ alkyl, C₁₋₄ haloalkyl, C₁₋₄ cyanoalkyl, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}, wherein said C₁₋₆ alkyl is optionally substituted by 1, 2 or 3 substituents independently selected from CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}S(O)₂R^{b2}, S(O)₂R^{b2}, and S(O)₂NR^{c2}R^{d2}.

In some embodiments, R¹ is H.

In some embodiments, Z is a group of Formula (Z-1):

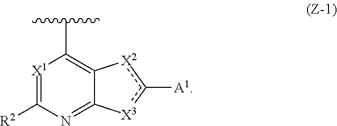

In some embodiments, Z is a group of Formula (Z-1a):

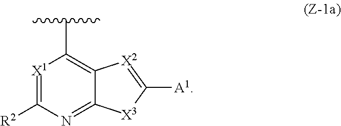

In some embodiments, Z is a group of Formula (Z-1b):

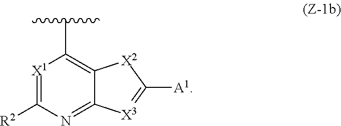

In some embodiments, X¹ is N.

In some embodiments, X² is selected from CR⁸ and N.

In some embodiments, X² is selected from CR⁸.

In some embodiments, X² is selected from CH.

In some embodiments, X² is selected from N.

In some embodiments, X³ is selected from CH, S, and NR^{N};

In some embodiments, X³ is selected from S and NR^{N}.

In some embodiments, X³ is selected from S, O, and NR^{N}.

In some embodiments, X³ is NR^{N}.

In some embodiments, X³ is S.

In some embodiments, R² is H.

In some embodiments, A¹ is selected from H, halo, C₁₋₄ alkyl, C₁₋₄ haloalkyl, C₁₋₄ haloalkoxy, CN, NO₂, OR^{a4}, C(O)R^{b4}, C(O)NR^{c4}R^{d4}, C(O)OR^{a4}, NR^{c4}R^{d4}, NR^{c4}C(O)R^{b4}, NR^{c4}S(O)₂R^{b4}, S(O)₂R^{b4}, and S(O)₂NR^{c4}R^{d4}, wherein said C₁₋₄ alkyl, is optionally substituted by 1, 2 or 3 substituents independently selected from CN, NO₂, OR^{a4}, C(O)R^{b4}, C(O)NR^{c4}R^{d4}, C(O)OR^{a4}, NR^{c4}R^{d4}, NR^{c4}C(O)R^{b4}, NR^{c4}S(O)₂R^{b4}, S(O)²R^{b4}, and S(O)²NR^{c4}R^{d4}.

In some embodiments, A¹ is selected from H, C₁₋₄ alkyl, C₁₋₄ haloalkyl, and C₁₋₄ haloalkoxy.

In some embodiments, A¹ is C₁₋₄ haloalkyl.

In some embodiments, A¹ is 2,2,2-trifluoroethyl.

In some embodiments, A¹ is 2,2-difluoroethyl.

In some embodiments, Z is a group of Formula (Z-2):

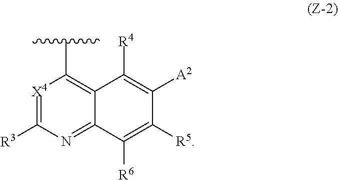

In some embodiments, X⁴ is CR⁹.

In some embodimetns, X⁴ is CH.

In some embodiments, X⁴ is N.

In some embodiments, A² is selected from H, halo, C₁₋₄ alkyl, C₁₋₄ haloalkyl, C₁₋₄ haloalkoxy, CN, NO₂, OR^{a4}, C(O)R^{b4}, C(O)NR^{c4}R^{d4}, C(O)OR^{a4}, NR^{c4}R^{d4}, NR^{c4}C(O)R^{b4}, NR^{c4}S(O)₂R^{b4}, S(O)₂R^{b4}, and S(O)₂NR^{c4}R^{d4}, wherein said C₁₋₄ alkyl, is optionally substituted by 1, 2 or 3 substituents independently selected from CN, NO₂, OR^{a4}, C(O)R^{b4}, C(O)NR^{c4}R^{d4}, C(O)OR^{a4}, NR^{c4}R^{d4}, NR^{c4}C(O)R^{b4}, NR^{c4}S(O)₂R^{b4}, S(O)₂R^{b4}, and S(O)₂NR^{c4}R^{d4}.

In some embodiments, A² is selected from H, C₁₋₄ alkyl, C₁₋₄ haloalkyl, and C₁₋₄ haloalkoxy.

In some embodiments, A² is C₁₋₄ haloalkyl.

In some embodiments, A² is 2,2,2-trifluoroethyl.

In some embodiments, A² is 2,2-difluoroethyl. In some embodiments, R³, R⁴, R⁵ and R⁶ are each independently selected from H, C₁₋₃ alkyl and C₁₋₃ haloalkyl.

In some embodiments, R³, R⁴, R⁵ and R⁶ are each H.

In some embodiments, the compounds of the invention have Formula (IIa), (IIb), (IIc), (IId), (IIe), (IIf), (IIg), (IIg), (IIi), or (IIj):

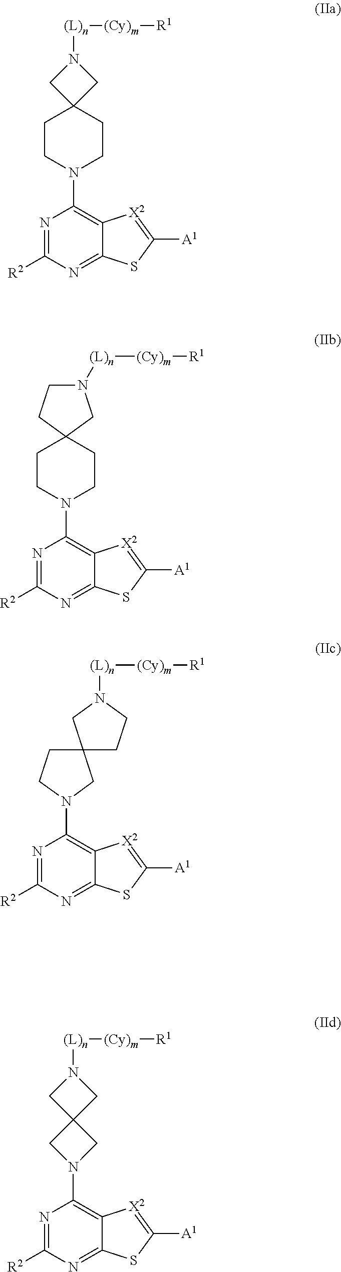

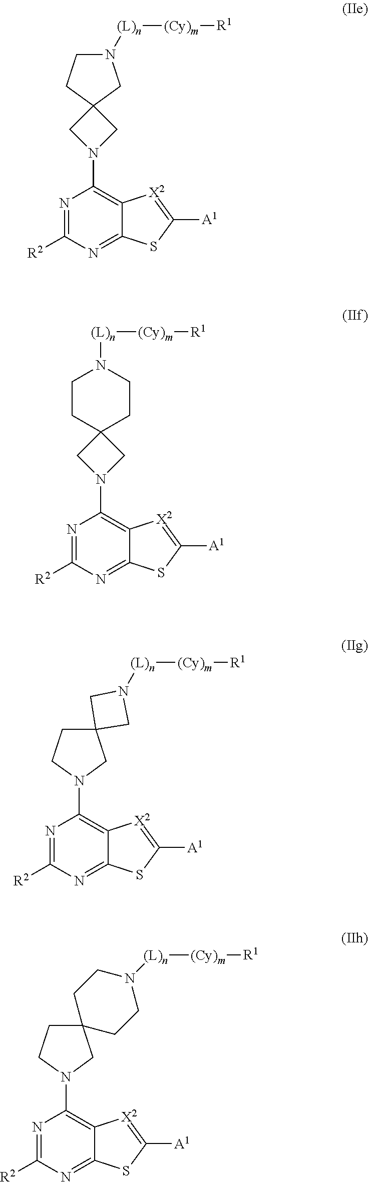

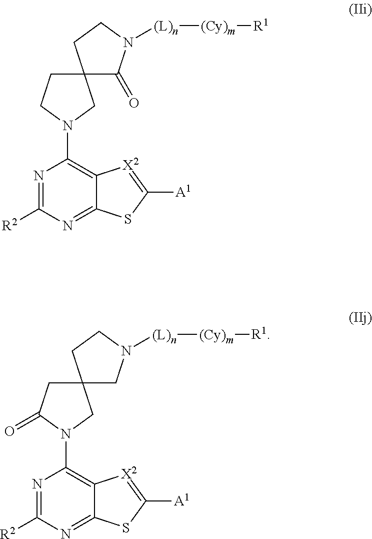

In some embodiments, the compounds of the invention have Formula (IIIa), (IIIb), or (IIIc):

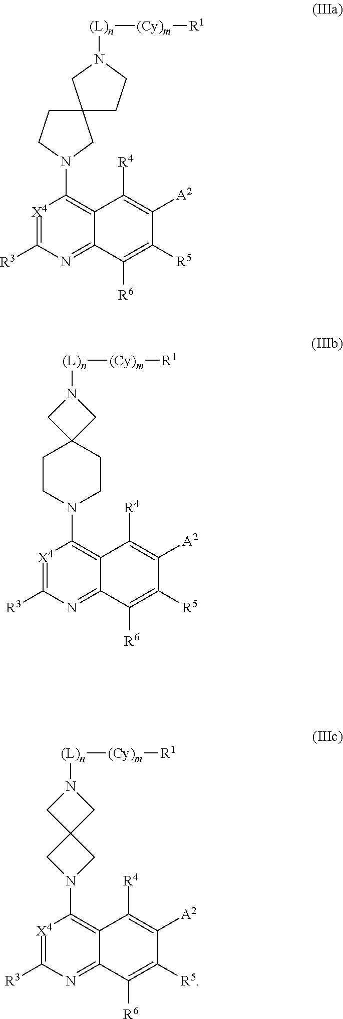

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. As used herein, the term “substituted” means that a hydrogen atom is removed and replaced by a substituent. The term “substituted” may also mean that two hydrogen atoms are removed and replaced by a divalent substituent such as an oxo or sulfide group. It is to be understood that substitution at a given atom is limited by valency.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

The term “z-membered” (where z is an integer) typically describes the number of ring-forming atoms in a moiety where the number of ring-forming atoms is z. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, linking substituents are described. It is specifically intended that each linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the term “Cᵢ₋ⱼ alkyl,” employed alone or in combination with other terms, refers to a saturated hydrocarbon group that may be straight-chain or branched, having i to j carbons. In some embodiments, the alkyl group contains from 1 to 6 carbon atoms, or from 1 to 4 carbon atoms, or from 1 to 3 carbon atoms. Examples of alkyl moieties include, but are not limited to, chemical groups such as methyl, ethyl, n-propyl, isopropyl, n-butyl, s-butyl, and t-butyl. In some embodiments, where an alkyl group is a linking group, it may be refered to as “Cᵢ₋ⱼ alkylene.”

As used herein, the term “Cᵢ₋ⱼ alkoxy,” employed alone or in combination with other terms, refers to a group of formula —O-alkyl, wherein the alkyl group has i to j carbons. Example alkoxy groups include methoxy, ethoxy, and propoxy (e.g., n-propoxy and isopropoxy). In some embodiments, the alkyl group has 1 to 3 carbon atoms.

As used herein, “Cᵢ₋ⱼ alkenyl,” employed alone or in combination with other terms, refers to an unsaturated hydrocarbon group having one or more double carbon-carbon bonds and having i to j carbons. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “Cᵢ₋ⱼ alkynyl,” employed alone or in combination with other terms, refers to an unsaturated hydrocarbon group having one or more triple carbon-carbon bonds and having i to j carbons. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, the term “Cᵢ₋ⱼ alkylamino,” employed alone or in combination with other terms, refers to a group of formula —NH(alkyl), wherein the alkyl group has i to j carbon atoms. In some embodiments, the alkyl group has 1 to 6 or 1 to 4 carbon atoms.

As used herein, the term “di-Cᵢ₋ⱼ-alkylamino,” employed alone or in combination with other terms, refers to a group of formula —N(alkyl)₂, wherein each of the two alkyl groups has, independently, i to j carbon atoms. In some embodiments, each alkyl group independently has 1 to 6 or 1 to 4 carbon atoms. In some embodiments, the dialkylamino group is —N(C₁₋₄ alkyl)₂ such as, for example, dimethylamino or diethylamino.

As used herein, the term “Cᵢ₋ⱼ alkylthio,” employed alone or in combination with other terms, refers to a group of formula —S-alkyl, wherein the alkyl group has i to j carbon atoms. In some embodiments, the alkyl group has 1 to 6 or 1 to 4 carbon atoms. In some embodiments, the alkylthio group is C₁₋₄ alkylthio such as, for example, methylthio or ethylthio.

As used herein, the term “thiol,” employed alone or in combination with other terms, refers to —SH.

As used herein, the term “amino,” employed alone or in combination with other terms, refers to a group of formula —NH₂.

As used herein, “Cᵢ₋ⱼ haloalkoxy,” employed alone or in combination with other terms, refers to a group of formula —O-haloalkyl having i to j carbon atoms. An example haloalkoxy group is OCF₃. An additional example haloalkoxy group is OCHF₂. In some embodiments, the haloalkoxy group is fluorinated only. In some embodiments, the alkyl group has 1 to 6 or 1 to 4 carbon atoms. In some embodiments, the haloalkoxy group is C₁₋₄ haloalkoxy.

As used herein, the term “halo,” employed alone or in combination with other terms, refers to a halogen atom selected from F, Cl, I or Br. In some embodiments, “halo” refers to a halogen atom selected from F, Cl, or Br. In some embodiments, the halo substituent is F.

As used herein, the term “Cᵢ₋ⱼ haloalkyl,” employed alone or in combination with other terms, refers to an alkyl group having from one halogen atom to 2s+1 halogen atoms which may be the same or different, where “s” is the number of carbon atoms in the alkyl group, wherein the alkyl group has i to j carbon atoms. In some embodiments, the haloalkyl group is fluorinated only. In some embodiments, the haloalkyl group is fluoromethyl, difluoromethyl, or trifluoromethyl. In some embodiments, the haloalkyl group is trifluoromethyl. In some embodiments, the haloalkyl group is 2,2,2-trifluoroethyl. In some embodiments, the haloalkyl group is 2,2-difluoroethyl. In some embodiments, the haloalkyl group has 1 to 6 or 1 to 4 carbon atoms.

As used herein, “Cᵢ₋ⱼ cyanoalkyl,” employed alone or in combination with other terms, refers to a group of formula CN—(Cᵢ₋ⱼ alkyl)-.

As used herein, the term “aryl,” employed alone or in combination with other terms, refers to a monocyclic or polycyclic (e.g., having 2, 3 or 4 fused rings) aromatic hydrocarbon, such as, but not limited to, phenyl, 1-naphthyl, 2-naphthyl, anthracenyl, phenanthrenyl, and the like. In some embodiments, aryl is C₆₋₁₀ aryl. In some embodiments, aryl is C₆₋₁₄ aryl. In some embodiments, the aryl group is a naphthalene ring or phenyl ring. In some embodiments, the aryl group is phenyl.

As used herein, the term “Cᵢ₋ⱼ cycloalkyl,” employed alone or in combination with other terms, refers to a non-aromatic cyclic hydrocarbon moiety having i to j ring-forming carbon atoms, which may optionally contain one or more alkenylene groups as part of the ring structure. Cycloalkyl groups can include mono- or polycyclic ring systems. Polycyclic ring systems can include fused ring systems and spirocycles. Also included in the definition of cycloalkyl are moieties that have one or more aromatic rings fused (i.e., having a bond in common with) to the cycloalkyl ring, for example, benzo or pyrido derivatives of cyclopentane, cyclopentene, cyclohexane, and the like. A heterocycloalkyl group that includes a fused aromatic (e.g., aryl or heteroaryl) moiety can be attached to the molecule through an atom from either the aromatic or non-aromatic portion. One or more ring-forming carbon atoms of a cycloalkyl group can be oxidized to form carbonyl linkages. In some embodiments, cycloalkyl is C₃₋₁₀ cycloalkyl, C₃₋₇ cycloalkyl, or C₅₋₆ cycloalkyl. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. Further exemplary cycloalkyl groups include cyclopropyl, cyclobutyl, cyclopentyl, and cyclohexyl. Additional example cycloalkyl groups, where the cycloalkyl group has a fused aryl or heteroaryl moiety, include tetrahydronaphthalen-2-yl, 2,3-dihydro-1H-inden-2-yl; 2,3,4,9-tetrahydro-1H-carbazol-7-yl; 2,6,7,8-tetrahydrobenzo[cd]indazol-4-yl; and 5,6,7,8,9,10-hexahydrocyclohepta[b]indol-3-yl.

As used herein, the term “heteroaryl,” employed alone or in combination with other terms, refers to a monocyclic or polycyclic (e.g., having 2, 3 or 4 fused rings) aromatic heterocylic moiety, having one or more heteroatom ring members selected from nitrogen, sulfur and oxygen. In some embodiments, the heteroaryl group has 1, 2, 3, or 4 heteroatom ring members. In some embodiments, the heteroaryl group has 1, 2, or 3 heteroatom ring members. In some embodiments, the heteroaryl group has 1 or 2 heteroatom ring members. In some embodiments, the heteroaryl group has 1 heteroatom ring member. In some embodiments, the heteroaryl group is 5- to 10-membered or 5- to 6-membered. In some embodiments, the heteroaryl group is 5-membered. In some embodiments, the heteroaryl group is 6-membered. In some embodiments, the heteroaryl group is 9- or 10-membered bicyclic. In some embodiments, the heteroaryl is 9-membere bicyclic. When the heteroaryl group contains more than one heteroatom ring member, the heteroatoms may be the same or different. The nitrogen atoms in the ring(s) of the heteroaryl group can be oxidized to form N-oxides. Example heteroaryl groups include, but are not limited to, pyridinyl, pyrimidinyl, pyrazinyl, pyridazinyl, pyrrolyl, pyrazolyl, azolyl, oxazolyl, isoxazolyl, thiazolyl, isothiazolyl, imidazolyl, furanyl, thiophenyl, triazolyl, tetrazolyl, thiadiazolyl, quinolinyl, isoquinolinyl, indolyl, benzothiopheneyl, benzofuranyl, benzisoxazolyl, benzoimidazolyl, imidazo[1, 2-b]thiazolyl, purinyl, triazinyl, and the like. In some embodiments, the heteroaryl group is 9H-carbazol-2-yl; 1H-benzo[d]imidazol-6-yl; 1H-indol-6-yl; 1H-indazol-6-yl; 2H-indazol-4-yl; 1H-benzo[d][1,2,3]triazol-6-yl; benzo[d]oxazol-2-yl; quinolin-6-yl; or benzo[d]thiazol-2-yl.

As used herein, the term “heterocycloalkyl,” employed alone or in combination with other terms, refers to a non-aromatic heterocyclic ring system, which may optionally contain one or more unsaturations as part of the ring structure, and which has at least one heteroatom ring member independently selected from nitrogen, sulfur and oxygen. In some embodiments, the heterocycloalkyl group has 1, 2, 3, or 4 heteroatom ring members. In some embodiments, the heterocycloalkyl group has 1, 2, or 3 heteroatom ring members. In some embodiments, the heterocycloalkyl group has 1 or 2 heteroatom ring members. In some embodiments, the heterocycloalkyl group has 1 heteroatom ring member. When the heterocycloalkyl group contains more than one heteroatom in the ring, the heteroatoms may be the same or different. Example ring-forming members include CH, CH₂, C(O), N, NH, O, S, S(O), and S(O)₂. Heterocycloalkyl groups can include mono- or polycyclic (e.g., having 2, 3 or 4 fused rings) ring systems. Polycyclic rings can include both fused systems and spirocycles. Also included in the definition of heterocycloalkyl are moieties that have one or more aromatic rings fused (i.e., having a bond in common with) to the non-aromatic ring, for example, 1,2,3,4-tetrahydro-quinoline, dihydrobenzofuran and the like. A heterocycloalkyl group that includes a fused aromatic moiety can be attached to the molecule through an atom from either the aromatic or non-aromatic portion. The carbon atoms or heteroatoms in the ring(s) of the heterocycloalkyl group can be oxidized to form a carbonyl, sulfinyl, or sulfonyl group (or other oxidized linkage) or a nitrogen atom can be quaternized. In some embodiments, heterocycloalkyl is 5- to 10-membered, 4- to 10-membered, 4- to 7-membered, 5-membered, or 6-membered. Examples of heterocycloalkyl groups include 1,2,3,4-tetrahydro-quinolinyl, dihydrobenzofuranyl, azetidinyl, azepanyl, pyrrolidinyl, piperidinyl, piperazinyl, morpholinyl, thiomorpholinyl, and pyranyl. Examples of heterocycloalkyl groups that include one or more fused aromatic groups (e.g., aryl or heteroaryl) include N-(2′-oxospiro[cyclohexane-1,3′-indolin]-6′-yl; 1,2,3,4-tetrahydroisoquinolin-6-yl; 2,3-dihydro-1H-benzo[d]imidazol-5-yl; 1,3-dihydrospiro[indene-2,3′-indolin]-6′-yl; 2,3-dihydrobenzo[d]oxazol-5-yl; 1,2-dihydroquinolin-7-yl; indolin-6-yl; spiro[cyclopentane-1,3′-indolin]-6′-yl; spiro[cyclohexane-1,3′-indolin]-6′-yl; chroman-6-yl; 3,4-dihydro-2H-benzo[b][1,4]oxazin-6-yl; and benzo[d][1,3]dioxol-5-yl.

As used herein, the term “arylalkyl,” employed alone or in combination with other terms, refers to an alkyl group substituted by an aryl group.

As used herein, the term “cycloalkylalkyl,” employed alone or in combination with other terms, refers to an alkyl group substituted by a cycloalkyl group.

As used herein, the term “heteroarylalkyl,” employed alone or in combination with other terms, refers to an alkyl group substituted by a heteroaryl group.

As used herein, the term “hetercycloalkylalkyl,” employed alone or in combination with other terms, refers to an alkyl group substituted by a heterocycloalkyl group.

As used herein, the term “Cᵢ₋ⱼ alkylsulfinyl,” employed alone or in combination with other terms, refers to a group of formulat —S(═O)—(Cᵢ₋ⱼ alkyl).

As used herein, the term “Cᵢ₋ⱼ alkylsulfinyl,” employed alone or in combination with other terms, refers to a group of formulat —S(═O)₂—(Cᵢ₋ⱼ alkyl).

As used herein, the term “carboxy,” employed alone or in combination with other terms, refers to a —C(═O)OH group.

As used herein, the term “Cᵢ₋ⱼ alkylcarbonyl,” employed alone or in combination with other terms, refers to a group of formula —C(═O)—(Cᵢ₋ⱼ alkyl).

As used herein, the term “Cᵢ₋ⱼ alkoxycarbonyl,” employed alone or in combination with other terms, refers to a group of formula —C(═O)O—(Cᵢ₋ⱼ alkyl).

The compounds described herein can be asymmetric (e.g., having one or more stereocenters). All stereoisomers, such as enantiomers and diastereoisomers, are intended unless otherwise indicated. Where a compound name or structure is silent with respect to the stereochemistry of a stereocenter, all possible configurations at the stereocenter are intended. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

When the compounds of the invention contain a chiral center, the compounds can be any of the possible stereoisomers. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

When a disclosed compound is named or depicted without indicating the stereochemistry of one or more stereocenters, each of the stereoisomers resulting from the possible stereochemistries at the undefined stereocenter(s) are intended to be encompassed. For example, if a stereocenter is not designated as R or S, then either or both are intended.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. Isotopes of constituent atoms of the compounds of the invention can be present in natural or non-natural abundance. Examples of isotopes of hydrogen include deuterium and tritium. In some embodiments, the compounds of the invention are deuterated, meaning at least one deuterium atom is present in the place of a hydrogen atom. In some embodiments, 1, 2, 3, 4, 5, 6, 7, or 8 hydrogens in a compound of the invention are replaced by deuterium. Methods for replacing hydrogen with deuterium in a molecule are known in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified (e.g., in the case of purine rings, unless otherwise indicated, when the compound name or structure has the 9H tautomer, it is understood that the 7H tautomer is also encompassed).

All compounds, and pharmaceutically acceptable salts thereof, can be found together with other substances such as water and solvents (e.g., hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in a compound of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19, and in Stahl et al., Handbook of Pharmaceutical Salts: Properties, Selection, and Use, (Wiley, 2002).

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject or patient is a human in need of treatment.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups (“Pg”), can be readily determined by one skilled in the art. The chemistry of protecting groups (“Pg”) can be found, for example, in P. G. M. Wuts and T. W. Greene, Protective Groups in Organic Synthesis, 4^{th} Ed., Wiley & Sons, Inc., New York (2006), which is incorporated herein by reference in its entirety.

Compounds of the invention can be prepared employing conventional methods that utilize readily available reagents and starting materials. The reagents used in the preparation of the intermediates of this invention can be either commercially obtained or can be prepared by standard procedures described in the literature. Various technologies such as solid phase chemistry, microwave chemistry or flow chemistry etc., can also be utilized to synthesize intermediates or final compounds. Furthermore, other methods of preparing compounds of the invention will be readily apparent to person of ordinary skill in the art in light of the following reaction and schemes and examples. Unless otherwise indicated all the variables are defined below. Suitable method of synthesis are described in the following references: March, Advanced Organic Chemistry, 3^{rd} edition, John Wiley & Sons, 1985; Greene and Wuts, Protective Groups in Organic Chemistry, 2^{nd} edition, John Wiley & Sons 1991; and Larock, Comprehensive Organic Transformations, 4^{th} edition, VCH publishers Inc., 1989. Furthermore, in any one synthesis, one or more of the reagents, intermediates or chemicals may be used in excess amount to ensure the completion of reaction. Suitable reaction temperatures generally range from about 0° C. to about the boiling point of the solvent. More typically, temperatures are sufficiently high to allow refluxing, for example, about 68° C. for tetrahydrofuran. In some cases, such as microwave conditions, the temperature of the reaction may exceed the boiling point of the solvent.

The compounds of the invention can be synthesized by the methods described in Schemes 1-3 below. Many of the synthetic steps are well described in as in F. A. Carey, R. J. Sundberg, Advanced Organic Chemistry, 2^{nd} ed., Plenum publication in 1983. The synthesis of various hydroxyl-substituted heterocycles (1) is well documented in the literature and can be synthesized by known literature methods. The general synthesis of useful heterocyclic rings are referenced in The Handbook of Heterocyclic Chemistry, Alan R. Katritzky; Pergamon Press, NY, USA, 1^{st} ed. 1986. The depicted intermediates may also available as commercial reagents from numerous vendors.

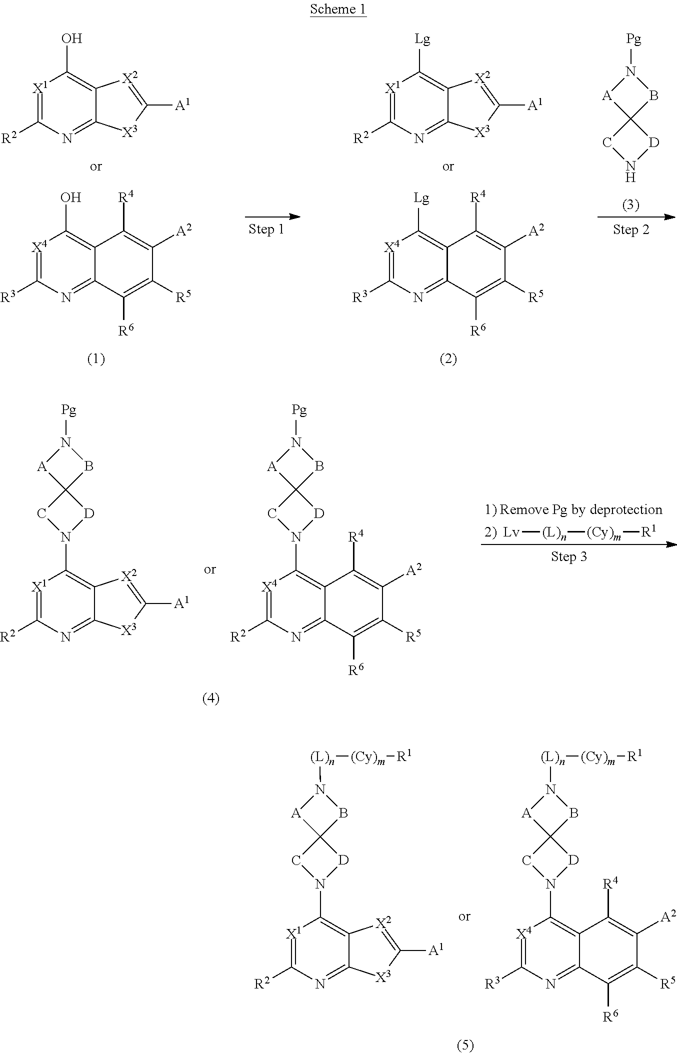

Scheme 1, Step 1: The OH of an heterocyclic alcohol (or amide) (1) can be converted to a suitable leaving group for nucleophilic reaction or for metal-catalyzed coupling. See, for example, Comprehensive Organic Transformation, R. Larock, 1^{st} edition, 1989, VCH publications, NY, USA for various methods of transformation. For example, the hydroxy moiety of (1) can be converted into a leaving group (Lg) or a coupling partner such a sulphate (e.g., mesylate, triflate, etc.) which can be achieved by reaction of (1) with a phosphorous chloride or sulfonyl chloride reagent in various aprotic or halogenated solvents. For example, (1) can be treated with phosphorous trichloride or thionyl chloride neat or in halogenated solvents as dichoromethane or 1,2-dichloroethane.

Scheme 1, Step 2: The reaction of intermediate (2) with various spirocyclic di-amines can be accomplished by numerous methods as described in Advanced Organic Chemistry, Jerry March, 3^{rd} edition, John Wiley & sons, 1985. An example method involves reacting protected spirocyclic di-amines (3) with heterocycles (2) in the presence of base. Optimally, one nitrogen of the spiro-diamines (3) is protected with a nitrogen protecting group (Pg) such as Boc. Other suitable protecting groups are described in Greene and Wuts Protective groups in Organic chemistry 2^{nd} edition, John Wiley & sons 1991. The reaction can be carried out in aprotic solvents such as halogenated solvents (e.g., dichloromethane, dichloroethane etc), or oxygentated solvents (e.g., ethers, dimethyl formamide, etc.). The bases can be, for example, inorganic alkali or alkaline salts of carbonates or tri-substituted amine analogs such as triethyl amine, or pyridine etc.

Scheme 1, Step 3. The nitrogen protecting group (Pg) of (4) can be removed, for example, with various strong acids in the presence of polar aprotic solvents such as ethers, or halogenated solvents like dichloromethane. For example, removal of tert-butoxycarbonyl group (Boc) can be carried out by use of a strong acid such as trifluoroacetic acid in dichloromethane or use of HCl gas in aprotic ether solvents such 1,4-dioxane or tetrahydrofuran, etc. After deprotection (removal of Pg), compounds of the invention can be prepared by reaction of the unprotected amine with electophiles Lv-(L)ₙ-(Cy)ₘ-R¹, where Lv is a leaving group such as halo, or the combination of Lv and L forms an electrophilic moiety such as an aldehyde or ketone. Suitable example reactions are described in March, Advanced Organic Chemistry, 3^{rd} edition, John Wiley & Sons, 1985. Similarly, the spirocylcic di-amine can be reacted with aryl halides to provide arylated amines. The spirocyclic amines can also be reacted with various other electrophiles such as isocyanates, sulfonyl chlorides, etc. to form compounds of the invention (5).

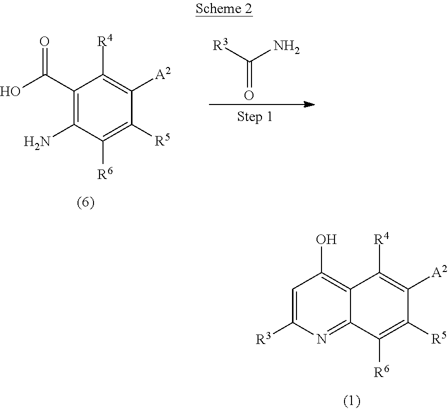

Scheme 2: Bicyclic intermediates (1) can be prepared by reaction of (6) with formamide, or a derivative thereof, in appropriate protic or aprotic solvents as referenced in Journal of Chemical Research, Synopses, (7), 214-15; 1985 by John M. Barker, et al. or Bioorganic & Medicinal Chemistry, 22(21), 6146-6155; 2014 by Wei Yang, et al.

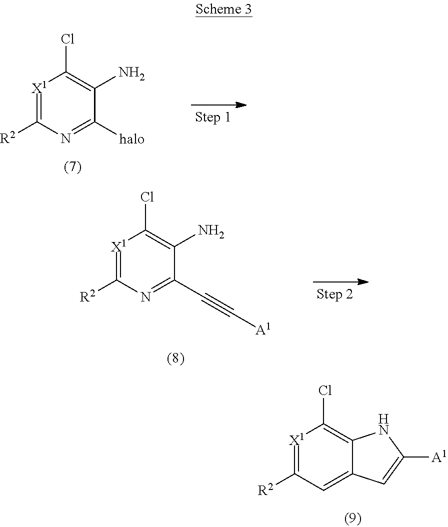

Scheme 3, Step 1: The halo group of (7) can be convereted directly into a substituted alkyne by reaction with appropriate alkynes. Such methods are well described in Comprehensive Organic Transformation, by R. Larock, 1^{st} edition, 1989, VCH publications, NY, USA and also in Organometallics as Catalysts in the Fine Chemical Industry by M. Beller, 1^{st} edition, 2005, Springer Publications.

Scheme 3, Step 2: The alkyne intermediate (8) can be converted into bicyclic intermediate (9) by treatment with, for example, base such as sodium hydroxide in protic solvents.

### Methods of Use

The compounds of the invention are inhibitors of the interaction of menin with MLL and MLL fusion proteins. In some embodiments, the present invention is directed to a method of inhibiting the interaction between menin and MLL or an MLL fusion protein by contacting menin and MLL or the MLL fusion protein with a compound of the invention. The contacting can be carried out in vitro or in vivo. In some embodiments, the compounds of the invention can bind to menin, thereby interfering with the binding of MLL to menin. In some embodiments, the present invention provides a method of inhibiting the activity of menin by contacting menin with a compound of the invention in the presence of MLL or an MLL fusion protein. In further embodiments, the present invention provides a method of inhibiting the binding of MLL or an MLL fusion protein to menin, comprising contacting menin with a compound of the invention in the presence of the MLL or MLL fusion protein.

The compounds of the invention are also useful in treating diseases associated with the menin-MLL interaction or menin-MLL fusion protein interaction. For example, diseases and conditions treatable according to the methods of the invention include cancer, such as leukemia, and other diseases or disorders mediated by the menin-MLL interaction or menin-MLL fusion protein interaction such as diabetes.

Accordingly, the compounds of the invention are believed to be effective against a broad range of cancers, including, but not limited to, hematological cancer (e.g., leukemia and lymphoma), bladder cancer, brain cancer (e.g., glioma), diffuse intrinsic pontine glioma (DIPG)), breast cancer (e.g., triple-negative breast cancer), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer, renal cancer (e.g., renal cell carcinoma), skin cancer, thyroid cancer (e.g., papillary thyroid carcinoma), testicular cancer, sarcoma (e.g., Ewing's sarcoma), and AIDS-related cancers.

In some embodiments, the specific cancers that may be treated by the compounds, compositions and methods described herein include cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodeimal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia), and other lymphatic or hematologic cancers including, for example, childhood leukemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Szary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by the compounds of the invention include mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Szary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), and Waldenstrom's macroglobulinemia.

In particular embodiments, compounds of the invention are used to treate leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

In some embodiments, diseases and conditions treatable with compounds of the invention include insulin resistance, pre-diabetes, diabetes (e.g., Type 2 diabetes or Type 1 diabetes), and risk of diabetes. In some embodiments, diseases and conditions treatable with compounds of the invention include hyperglycemia. In some embodiments, the hyperglycemia is associated with diabetes, such as Type 2 diabetes. In some embodiments, compounds of the invention are used to treat loss of response to other anti-diabetic agents and/or reduced beta cell function in a patient or subject. In some embodiments, compounds of the invention are used to restore response to other anti-diabetic agents and/or to restore beta cell function and/or to reduce the need for insulin in a patient or subject. In some embodiments, compounds of the invention are used to reduce insulin resistance, reduce the risk of diabetes, or reduce increases in blood glucose caused by a statin in a subject taking a statin. In some embodiments, compounds of the invention are used to treat diabetes in a subject taking a statin or to prevent diabetes in a subject taking a statin. Methods of the invention include decreasing, reducing, inhibiting, suppressing, limiting or controlling in the patient elevated blood glucose levels. In further aspects, methods of the invention include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rousuvastatin and simvastatin.

In some embodiments, a patient is treated with (e.g., administered) a compound of the present invention in an amount sufficient to treat or ameliorate one or more of the diseases and conditions recited above (e.g., a therapauetically effective amount). The compounds of the invention may also be useful in the prevention of one or more of the diseases recited therein.

### Combination Therapy

The invention further relates to a combination therapy for treating a disease or a disorder described herein. In some embodiments, the combination therapy comprises administering at least one compound of the present invention in combination with one or more other pharmaceutically active agents for treating cancer or other disorders mediated by menin/MLL. In some embodiments, the combination therapy comprises administering at least one compound of the present invention in combination with one or more other pharmaceutically active agents, such as for the treatment of cancer. The pharmaceutically active agents can be combined with a compound of the invemtion in a single dosage form, or the therapeutics can be administered simultaneously or sequentially as separate dosage forms.

The compounds according to the invention may also be used in combination with immunotherapies, including but not limited to cell-based therapies, antibody therapies and cytokine therapies, for the treatment of a disease or disorder disclosed herein.

In certain embodiments, compounds according to the invention are used in combination with one or more passive immunotherapies, including but not limited to naked monoclonal antibody drugs and conjugated monoclonal antibody drugs. Examples of naked monoclonal antibody drugs that can be used include, but are not limited to rituximab (Rituxan®), an antibody against the CD20 antigen; trastuzumab (Herceptin®), an antibody against the HER2 protein; alemtuzumab (Lemtrada®, Campath®), an antibody against the CD52 antigen; cetuximab (Erbitux®), an antibody against the EGFR protein; and bevacizumab (Avastin®) which is an anti-angiogenesis inhibitor of VEGF protein.

Examples of conjugated monoclonal antibodies that can be used include, but are not limited to, radiolabeled antibody ibritumomab tiuxetan (Zevalin®); radiolabeled antibody tositumomab (Bexxar®); and immunotoxin gemtuzumab ozogamicin (Mylotarg®) which contains calicheamicin; BL22, an anti-CD22 monoclonal antibody-immunotoxin conjugate; radiolabeled antibodies such as OncoScint® and ProstaScint®; brentuximab vedotin (Adcetris®); ado-trastuzumab emtansine (Kadcyla®, also called TDM-1).

Further examples of therapeutic antibodies that can be used include, but are not limited to, REOPRO® (abciximab), an antibody against the glycoprotein IIb/IIIa receptor on platelets; ZENAPAX® (daclizumab) an immunosuppressive, humanized anti-CD25 monoclonal antibody; PANOREX™, a murine anti-17-IA cell surface antigen IgG2a antibody; BEC2, a murine anti-idiotype (GD3 epitope) IgG antibody; IMC-C225, a chimeric anti-EGFR IgG antibody; VITAXIN™ a humanized anti-αVβ3 integrin antibody; Campath 1H/LDP-03, a humanized anti CD52 IgG1 antibody; Smart M195, a humanized anti-CD33 IgG antibody; LYMPHOCIDE™, a humanized anti-CD22 IgG antibody; LYMPHOCIDE™ Y-90; Lymphoscan; Nuvion® (against CD3; CM3, a humanized anti-ICAM3 antibody; IDEC-114 a primatized anti-CD80 antibody; IDEC-131 a humanized anti-CD40L antibody; IDEC-151 a primatized anti-CD4 antibody; IDEC-152 a primatized anti-CD23 antibody; SMART anti-CD3, a humanized anti-CD3 IgG; 5G1.1, a humanized anti-complement factor 5 (C5) antibody; D2E7, a humanized anti-TNF-α antibody; CDP870, a humanized anti-TNF-α Fab fragment; IDEC-151, a primatized anti-CD4 IgG1 antibody; MDX-CD4, a human anti-CD4 IgG antibody; CD20-streptdavidin (+biotin-yttrium 90); CDP571, a humanized anti-TNF-α IgG4 antibody; LDP-02, a humanized anti-α4β7 antibody; OrthoClone OKT4A, a humanized anti-CD4 IgG antibody; ANTOVA™, a humanized anti-CD40L IgG antibody; ANTEGREN™, a humanized anti-VLA-4 IgG antibody; and CAT-152, a human anti-TGF-β₂ antibody.

In certain embodiments, compounds according to the invention are used in combination with one or more targeted immunotherapies containing toxins but not an antibody, including but not limited to denileukin diftitox (Ontak®), IL-2 linked to diphtheria toxin.

The compounds according to the invention may also be used in combination with adjuvant immunotherapies for the treatment of a disease or disorder disclosed herein. Such adjuvant immunotherapies include, but are not limited to, cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Gurin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of interleukins, for example IL-2, with other cytokines, such as IFN-alpha.

In certain embodiments, compounds according to the invention are used in combination with vaccine therapy, including but not limited to autologous and allogeneic tumor cell vaccines, antigen vaccines (including polyvalent antigen vaccines), dendritic cell vaccines, and viral vaccines.

In another embodiment, the present disclosure comprises administering to a subject with cancer an effective amount of a compound of the invention and one or more additional anti-cancer therapies selected from: surgery, anti-cancer agents/drugs, biological therapy, radiation therapy, anti-angiogenesis therapy, immunotherapy, adoptive transfer of effector cells, gene therapy or hormonal therapy. Examples of anti-cancer agents/drugs are described below.

In some embodiments, the anti-cancer agents/drug is, for example, adriamycin, aactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; palbociclib; Yervoy® (ipilimumab); Mekinist™ (trametinib); peginterferon alfa-2b, recombinant interferon alfa-2b; Sylatron™ (peginterferon alfa-2b); Tafinlar® (dabrafenib); Zelboraf® (vemurafenib); or nivolumab.

The compounds according to the present invention can be administered in combination with existing methods of treating cancers, for example by chemotherapy, irradiation, or surgery. Thus, there is further provided a method of treating cancer comprising administering an effective amount of a compound of the invention, or a pharmaceutically acceptable salt form thereof, to a subject in need of such treatment, wherein an effective amount of at least one additional cancer chemotherapeutic agent is administered to the subject. Examples of suitable cancer chemotherapeutic agents include any of: abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate.

In particular embodiments, compounds according to the invention are used in combination with one or more anti-cancer agent selected from methotrexate, paclitaxel albumin-stabilized nanoparticle formulation, ado-trastuzumab emtansine, eribulin, doxorubicin, fluorouracil, everolimus, anastrozole, pamidronate disodium, exemestane, capecitabine, cyclophosphamide, docetaxel, epirubicin, toremifene, fulvestrant, letrozole, gemcitabine, gemcitabine hydrochloride, goserelin acetate, trastuzumab, ixabepilone, lapatinib ditosylate, megestrol acetate, tamoxifen citrate, pamidronate disodium, palbociclib, and pertuzumab for the treatment of breast cancer.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclin-dependent kinase inhibitors; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-b enzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; zanoterone; zilascorb; zinostatin stimalamer; 5-fluorouracil; and leucovorin.

In some embodiments, the anti-cancer agent/drug is an agent that stabilizes microtubules. As used herein, a “microtubulin stabilizer” means an anti-cancer agent/drug which acts by arresting cells in the G2-M phases due to stabilization of microtubules. Examples of microtubulin stabilizers include ACLITAXEL® and Taxol® analogues. Additional examples of microtubulin stabilizers include without limitation the following marketed drugs and drugs in development: Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

In another embodiment, the anti-cancer agent/drug is an agent that inhibits microtubules. As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbott, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR-0Y-007 (National Health Research Institutes); SSR-250411 (Sanofi); Combretastatin A4; eribulin (Halaven®); and analogs and derivatives thereof.

In further embodiments, compounds according to the invention are used in combination with one or more alkylating agents, antimetabolites, natural products, or hormones.

Examples of alkylating agents useful in the methods of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.).

Examples of antimetabolites useful in the methods of the invention include but are not limited to folic acid analog (e.g., methotrexate), or pyrimidine analogs (e.g., fluorouracil, floxouridine, cytarabine), and purine analogs (e.g., mercaptopurine, thioguanine, pentostatin). Examples of natural products useful in the methods of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin) or enzymes (e.g., L-asparaginase).

Examples of hormones and antagonists useful for the treatment of cancer include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), and gonadotropin releasing hormone analog (e.g., leuprolide).

Other agents that can be used in combination with the compounds of the invention for the treatment of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), and adrenocortical suppressant (e.g., mitotane, aminoglutethimide). Other anti-cancer agents/drugs that can be used in combination with the compounds of the invention include, but are not limited to, liver X receptor (LXR) modulators, including LXR agonists and LXR beta-selective agonists; aryl hydrocarbon receptor (AhR) inhibitors; inhibitors of the enzyme poly ADP ribose polymerase (PARP), including olaparib, iniparib, rucaparib, veliparib; inhibitors of vascular endothelial growth factor (VEGF) receptor tyrosine kinases, including cediranib; programmed cell death protein 1 (PD-1) inhibitors, including nivolumab (Bristol-Myers Squibb Co.) and pembrolizumab (Merck & Co., Inc.; MK-3475); MEK inhibitors, including cobimetinib; B-Raf enzyme inhibitors, including vemurafenib; cytotoxic T lymphocyte antigen (CTLA-4) inhibitors, including tremelimumab; programmed death-ligand 1 (PD-L1) inhibitors, including MEDI4736 (AstraZeneca); inhibitors of the Wnt pathway; inhibitors of epidermal growth factor receptor (EGFR) including AZD9291 (AstraZeneca), erlotinib, gefitinib, panitumumab, and cetuximab; adenosine A2A receptor inhibitors; adenosine A2B receptor inhibitors; colony-stimulating factor-1 receptor (CSF1R) inhibitors, including PLX3397 (Plexxikon), and inhibitors of CD73.

The compounds of the invention can be used in combination with one or more therapeutic strategies including immune checkpoint inhibitors, including inhibitors of PD-1, PD-L1, and CTLA-4.

The compounds of the invention can be used in combination with one or more anti-cancer agents selected from MCL-1 inhibitors, e.g., homoharringtonin (HHT) and omacetaxine; BCL-2 inhibitors, e.g., venetoclax (ABT-199), navitoclax (ABT-263), ABT-737, gossypol (AT-101), apogossypolone (ApoG2) and obatoclax; selective inhibitors of nuclear export (SINEs), e.g., selinexor (KPT-330).

In particular embodiments, the compounds of the invention are used in combination with one or more anti-cancer agents selected from methotrexate (Abitrexate®; Folex®; Folex PFS®; Mexate®; Mexate-AQ®); nelarabine (Arranon®); blinatumomab (Blincyto®); rubidomycin hydrochloride or daunorubicin hydrochloride (Cerubidine®); cyclophosphamide (Clafen®; Cytoxan®; Neosar®); clofarabine (Clofarex®; Clolar®); cytarabine (Cytosar-U®; Tarabine PFS®); dasatinib (Sprycel®); doxorubicin hydrochloride; asparaginase Erwinia chrysanthemi (Erwinaze); imatinib mesylate (Gleevec®); ponatinib hydrochloride (Iclusig®); mercaptopurine (Purinethol; Purixan); pegaspargase (Oncaspar®); prednisone; vincristine sulfate (Oncovin®, Vincasar PFS®, Vincrex®); vincristine sulfate liposome (Marqibo®); hyper-CVAD (fractionated cyclophosphamide, vincristine, adriamycin, and dexamethasone); arsenic trioxide (Trisenox®); idarubicin hydrochloride (Idamycin®); mitoxantrone hydrochloride; thioguanine (Tabloid®); ADE (cytarabine, daunorubicin, and etoposide); alemtuzumab (Lemtrada®, Campath®); chlorambucil (Ambochlorin®, Amboclorin®, Leukeran®, Linfolizin®); ofatumumab (Arzerra®); bendamustine hydrochloride (Treanda®); fludarabine phosphate (Fludara®); obinutuzumab (Gazyva®); ibrutinib (Imbruvica®); idelalisib (Zydelig®); mechlorethamine hydrochloride (Mustargen®); rituximab (Rituxan®); chlorambucil-prednisone; CVP (cyclophosphamide, vincristine, and prednisone); bosutinib (Bosulif®); busulfan (Busulfex®; Myleran®); omacetaxine mepesuccinate (Synribo®); nilotinib (Tasigna®); Intron® A (recombinant interferon Alfa-2b); DOT1L inhibitors, including EPZ-5676 (Epizyme, Inc.); and inhibitors of bromodomain and extra-terminal motif (BET) proteins (BET inhibitors), including MS417, JQ1, I-BET 762, and I-BET 151 for the treatment of leukemia.

Compounds of the invention can be used in combination with one or more other agents or therapies for the treatment of insulin resistance, pre-diabetes, diabetes (e.g., Type 2 diabetes or Type 1 diabetes), and risk of diabetes, including but not limited to insulins and insulin analogues, such as Humulin® (Ell Lilly), Lantus® (Sanofi Aventis), Novolin® (Novo Nordisk), and Exubera® (Pfizer); Avandamet® (metformin HCI and rosiglitazone maleate, GSK); Avandaryl® (glimepiride and rosiglitazone maleate, GSK); Metaglip® (glipizide and metformin HCI, Bristol Myers Squibb); Glucovance® (glyburide and metformin HCI, Bristol Myers Squibb); PPAR gamma agonists, such as Avandia® (rosiglitizone maleate, GSK) and Actos® (pioglitazone hydrochloride, Takeda/Eli Lilly); sulfonylureas, such as Amaryl® (glimepiride, Sanofi Aventis), Diabeta® (glyburide, Sanofi Aventis), Micronase®/Glynase® (glyburide, Pfizer), and Glucotrol®/Glucotrol XL® (glipizide, Pfizer); meglitinides, such as Prandin®/NovoNorm® (repaglinide, Novo Nordisk), Starlix® (nateglinide, Novartis), and Glufast® (mitiglinide, Takeda); biguanides, such as Glucophase®/Glucophase XR® (metformin HCI, Bristol Myers Squibb) and Glumetza® (metformin HCI, Depomed); thiazolidinediones; amylin analogs; GLP-1 analogs; DPP-IV inhibitors such as Januvia® (sitagliptin, Merck) and Galvus® (vildagliptin, Novartis); PTB-1 B inhibitors; protein kinase inhibitors (including AMP-activated protein kinase inhibitors); glucagon antagonists, glycogen synthase kinase-3 beta inhibitors; glucose-6-phoshatase inhibitors; glycogen phosphorylase inhibitors; sodium glucose co-transporter inhibitors; and alpha-glucosidase inhibitors, such as Glycet® (miglitol, Pfizer); statins, fibrates, and Zetia® (ezetimibe); alpha-blockers; beta-blockers; calcium channel blockers; diuretics; angiotensin converting enzyme (ACE) inhibitors; dual ACE and neutral endopeptidase (NEP) inhibitors; angiotensin-receptor blockers (ARBs); aldosterone synthase inhibitors; aldosterone-receptor antagonists; endothelin receptor antagonists; orlistat; phentermine; sibutramine; Acomplia® (rimonabant); thiazolidinediones (e.g., rosiglitazone, pioglitazone); SGLT 2 inhibitors (e.g., dapagliflozin, remogliflozin etabonate, sergliflozin, canagliflozin, and 1-chloro-4-(β-D-glucopyranos-1-yl)-2-[4-((′S)-tetrahydrofuran-3-yloxy)-benzyl]-benzene); PPAR-gamma-agonists (e.g., Gl 262570) and antagonists; PPAR-gamma/alpha modulators (e.g., KRP 297); alpha-glucosidase inhibitors (e.g., acarbose, voglibose); DPPIV inhibitors (e.g., Januvia® (sitagliptin), Galvus®/Zomelis® (vildagliptin), Onglyza® (saxagliptin), Nesina®/Vipidia® (alogliptin), and Tradjenta®/Trajenta® (linagliptin)); alpha2-antagonists; glucagon-like protein-1 (GLP-1) receptor agonists and analogues (e.g., exendin-4); amylin; inhibitors of protein tyrosinephosphatase 1; substances that affect deregulated glucose production in the liver, e.g., inhibitors of glucose-6-phosphatase, or fructose-1,6-bisphosphatase, glycogen phosphorylase; glucagon receptor antagonists; inhibitors of phosphoenol pyruvate carboxykinase; glycogen synthase kinase and glucokinase activators; lipid lowering agents such as HMG-CoA-reductase inhibitors (e.g., simvastatin, atorvastatin); fibrates (e.g., bezafibrate, fenofibrate), nicotinic acid and the derivatives thereof, PPAR-alpha agonists, PPAR-delta agonists; ACAT inhibitors (e.g., avasimibe); cholesterol absorption inhibitors such as ezetimibe; bile acid-binding substances such as cholestyramine; inhibitors of ileac bile acid transport; HDL-raising compounds such as CETP inhibitors and ABC1 regulators; active substances for treating obesity such as sibutramine and tetrahydrolipostatin; SDRIs; axokine; leptin; leptin mimetics; antagonists of the cannabinoid 1 receptor; and MCH-1 receptor antagonists; MC4 receptor agonists; NPY5 and NPY2 antagonists; beta3 adrenergic agonists such as SB-418790 and AD-9677; agonists of the 5HT2c receptor; GABA-receptor antagonists; Na-channel blockers; topiramate; protein-kinase C inhibitors; advanced glycation end product inhibitors; and aldose reductase inhibitors.

### Pharmaceutical Formulations, Administration, and Dosage Forms

When employed as pharmaceuticals, the compounds of the invention can be administered in the form of a pharmaceutical composition which refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Compounds or compositions described herein may be administered to a patient using any amount and any route of administration effective for treating or lessening the severity of one or more of the diseases and conditions described herein. The exact amount required will vary from subject to subject, depending on the species, age, and general condition of the subject, the severity of the infection, disease or disorder, the particular agent, its mode of administration, and the like. Provided compounds are preferably formulated in a particular unit dosage form for ease of administration and uniformity of dosage. The expression “unit dosage form” as used herein refers to a physically discrete unit of agent appropriate for the patient to be treated.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### EXAMPLES

As depicted in the Examples below, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Microwave reactions were carried out in a CEM reactor using discovery SP system. Where NMR data are presented, spectra were obtained in Varian-400 (400 MHz). Spectra are reported as ppm downfield from tetramethylsilane with the number of proton, multiplicities and, in certain instances, coupling constants indicated parenthetically along with reference to deuterated solvent. Compounds were also purified by ISCO flash chromatography system utilizing standard methods described in the manual.

Compounds were purified by acidic or basic preparative HPLC method as described below.

### Preparative RP-HPLC Method A:

RP-HPLC (C-18, Boston Green ODS 150*30 mm*5 μm; eluent-gradient: water+0.1% TFA/acetonitrile=81:19 to 51:49)

Mobile phase A: water+0.1% TFA; Mobile phase B: CH₃CN; Flow rate: 30 mL/min; Detection: UV 220 nm/254 nm; Column: Boston Green ODS 150*30 mm*5 μm; Column temperature: 30° C.

| Time in min | % A | % B |
|---|---|---|
| 0.00 | 81 | 19 |
| 8.00 | 51 | 49 |
| 8.20 | 0 | 100 |
| 10.00 | 0 | 100 |

### Preparative RP-HPLC Method B:

RP-HPLC (C-18, Phenomenex Synergi C18 250*21.2 mm*4 μm; eluent-gradient: water+0.1% TFA/acetonitrile=75:25 to 45:55).

Mobile phase A: water+0.1% TFA; Mobile phase B: CH₃CN; Flow rate: 25 mL/min; Detection: UV 220 nm/254 nm; Column: Phenomenex Synergi C18 250*21.2 mm*4 μm; Column temperature: 30° C.

| Time in min | % A | % B |
|---|---|---|
| 0.00 | 75 | 25 |
| 10.00 | 45 | 55 |
| 10.20 | 0 | 100 |
| 12.00 | 0 | 100 |

### Preparative RP-HPLC Method C:

RP-HPLC (C-18, Phenomenex Synergi C18 250*21.2 mm*4 μm; eluent-gradient: water+0.05% HCl/acetonitrile=82:18 to 52:48).

Mobile phase A: water with 0.05% HCl; Mobile phase B: CH₃CN; Flow rate: 30 mL/min; Detection: UV 220 nm/254 nm; Column: Phenomenex Gemini 150*30 mm*4 μm; Column temperature: 30° C.

| Time in min | % A | % B |
|---|---|---|
| 0.00 | 82 | 18 |
| 8.00 | 52 | 48 |
| 8.20 | 0 | 100 |
| 10.00 | 0 | 100 |

### Preparative RP-HPLC Method D:

RP-HPLC (C-18, Phenomenex Gemini 150*25 mm*10 μm; eluent-gradient: water+0.05% ammonia hydroxide/acetonitrile=30:70 to 0:100).

Mobile phase A: water with 0.05% ammonia hydroxide; Mobile phase B: CH₃CN; Flow rate: 25 mL/min; Detection: UV 220 nm/254 nm; Column: Phenomenex Gemini 150*25 mm*10 μm; Column temperature: 30° C.

| Time in min | % A | % B |
|---|---|---|
| 0.00 | 30 | 70 |
| 8.00 | 0 | 100 |
| 8.20 | 0 | 100 |
| 10.00 | 0 | 100 |

### Preparative RP-HPLC Method E:

Mobile phase A: water with 0.1% TFA; Mobile phase B: acetonitrile with 0.1% TFA; Flow rate: 25 mL/min; Detection: UV 220 nm/254 nm; Column: C-18 Synergi Max-RP 150*30 mm*4 μm; Column temperature: 30° C.

| Time in min | % A | % B |
|---|---|---|
| 0.0 | 90 | 10 |
| 12.00 | 60 | 40 |
| 12.20 | 10 | 90 |
| 13.5 | 90 | 10 |

LCMS data were obtained by utilizing the following chromatographic conditions:

### LCMS Method A:

HPLC System: Waters ACQUITY; Column: Waters ACQUITY CSH™ C18 1.7 μM. Guard column: Waters Assy. Frit, 0.2 μM, 2.1 mm; Column temperature: 40° C.

Mobile Phase: A: TFA:Water (1:1000, v:v); Mobile phase B: TFA:ACN (1:1000, v:v); Flow Rate: 0.65 mL/min; Injection Volume: 2 μL; Acquisition time: approximately 1.5 min.

Gradient Program:

| Time (min) | B % |
|---|---|
| 0 | 10 |
| 1.0 | 90 |
| 1.20 | 10 |

Mass Spectrometer: Waters SQD; Ionization: Positive Electrospray Ionization (ESI); Mode Scan (100-1400 m/z in every 0.2 second); ES Capillary Voltage: 3.5 kV; ES Cone Voltage: 25 v.

Source Temperature: 120° C.; Desolvation Temperature: 500° C.; Desolvation Gas Flow: Nitrogen Setting 650 (L/h); Cone Gas Flow: Nitrogen Setting 50 (L/h).

### LCMS Method B:

HPLC System: Waters ACQUITY; Column: Waters ACQUITY CSH™ C18 1.7 μM. Guard column: Waters Assy. Frit, 0.2 μM, 2.1 mm; Column tem: 40° C.

Mobile Phase: A: TFA:Water (1:1000, v:v); Mobile phase B: TFA:ACN (1:1000, v:v); Flow Rate: 0.65 mL/min; Injection Volume: 2 μL; Acquisition time: approximately 1.5 min.

Gradient Program:

| Time (min) | B % |
|---|---|
| 0 | 10 |
| 2.0 | 90 |
| 2.20 | 10 |

Mass Spectrometer: Waters SQD; Ionization: Positive Electrospray Ionization (ESI); Mode Scan (100-1400 m/z in every 0.2 second); ES Capillary Voltage: 3.5 kV; ES Cone Voltage: 25 v.

Source Temperature: 120° C.; Desolvation Temperature: 500° C.; Desolvation Gas Flow: Nitrogen Setting 650 (L/h); Cone Gas Flow: Nitrogen Setting 50 (L/h).

### LCMS Method C:

| | |
|---|---|
| Column | MERCK, RP-18e 25-2 mm |
| Mobile Phase | A: water(4 L) + TFA(1.5 mL) |
| | B: acetonitrile(4 L) + TFA(0.75 mL) |

| TIME(min) | B % |
|---|---|
| 0 | 5 |
| 0.7 | 95 |
| 1.1 | 95 |
| 1.11 | 5 |
| 1.5 | 5 |

| | |
|---|---|
| Flow Rate | 1.5 mL/min |
| wavelength | UV 220 nm |
| Oven Temp | 50° C. |
| MS ionization | ESI |

### LCMS Method D:

| | |
|---|---|
| Column | Xbrige Shield RP-18, 5 μm, 2.1*50 mm |
| Mobile Phase | A: water(1 L) + NH3H2O(0.5 mL) |
| | B: acetonitrile |

| TIME(min) | B % |
|---|---|
| 0 | 10 |
| 2 | 80 |
| 2.48 | 80 |
| 2.49 | 10 |
| 3 | 10 |

| | |
|---|---|
| Flow Rate | 1.0 mL/min |
| wavelength | UV 220 nm |
| Oven Temp | 30° C. |
| MS ionization | ESI |

### LCMS Method E:

| | |
|---|---|
| Column | Xtimate C18 2.1*30 mm, 3 μm |
| Mobile Phase | A: water(4 L) + TFA(1.5 mL) |
| | B: acetonitrile(4 L) + TFA(0.75 mL) |

| TIME(min) | B % |
|---|---|
| 0 | 10 |
| 0.9 | 80 |
| 1.5 | 80 |
| 1.51 | 10 |
| 2 | 10 |

| | |
|---|---|
| Flow Rate | 1.2 mL/min |
| wavelength | UV 220 nm |
| Oven Temp | 50° C. |
| MS ionization | ESI |

### LCMS Method F:

| Column | Xtimate C18 2.1*30 mm, 3 μm | |
|---|---|---|
| Mobile Phase | A: water(4 L) + TFA(1.5 mL) | |
| | B: acetonitrile(4 L) + TFA(0.75 mL) | |
| | TIME(min) | B % |
| | 0 | 0 |
| | 0.9 | 60 |
| | 1.5 | 60 |
| | 1.51 | 0 |
| | 2 | 0 |
| Flow Rate | 1.2 mL/min | |
| wavelength | UV 220 nm | |
| Oven Temp | 50° C. | |
| MS ionization | ESI | |

The following are Supercritical Fluid Chromatography (SFC) separation methods for racemic compounds.

### Method A:

Instrument: Thar SFC 80; Column: AD 250 mm*30 mm, 5 μm; Mobile phase: A: Supercritical CO₂, B: IPA (0.05% DEA), A:B=80:20 at 60 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm.

### Method B:

Instrument: SFC MG2; Column: OJ 250 mm*30 mm, 5 μm; Mobile phase: A: Supercritical CO₂, B: MeOH (0.05% DEA), A:B=90:10 at 70 mL/min; Column Temp: 38° C.; Nozzle Pressure: 100 Bar Nozzle Temp: 60° C.; Evaporator Temp: 20° C.; Trimmer Temp: 25° C.; Wavelength: 220 nm.

The invention is illustrated by the following examples, in which the following abbreviations may be employed:

| Abbreviation | Meaning |
|---|---|
| ACN | acetonitrile |
| DCE | 1,2-dichloroethane |
| DCM | methylene chloride |
| DIEA | diisopropylethyl amine |
| DMA | dimethyl acetamide |
| DMF | dimethyl formamide |
| dppf | 1,1-bis(diphenylphosphino)ferrocene |
| Et₃N | triethylamine |
| EtOAc | ethyl acetate |
| EtOH | ethanol |
| h | hour(s) |
| HATU | 1-[bis(dimethylamino)methylene]-1H-1,2,3- |
| | triazolo[4,5-b]pyridinium 3-oxid |
| | hexafluorophosphate. |
| HBTU | 2-(1H-benzotriazole-1-yl)-1,1,3,3- |
| | tetramethyluronium hexafluorophosphate |
| HCl | hydrochloric acid |
| HPLC | high performance liquid chromatography |
| Im | imidazaole |
| KI | potassium iodide |
| LCMS | liquid chromatography-mass spectorphotmetry |
| min | minute(s) |
| Me | methyl |
| mL | milliliters |
| mmol | millimoles |
| mg | milligram |
| NaBH₃CN | sodium cyanoborohydride |
| RP | reverse phase |
| RT | room temperature |
| SFC | super critical fluid chromatography |
| t_{R} | retention time |
| TBAF | tetra butyl ammonium fluoride |
| TBDMS | tert butyl dimethyl silyl |
| TEA | triethylamine |
| TFA | trifluoroacetic acid |
| THF | tetrahydrofuran |
| TLC | thin layer chromatography |
| XPhos | dicyclohexyphosphino-2′,4′,6′-triiso-propyl- |
| | 1,1′-biphenyl |

### Example 1

### 4-(2-benzyl-2,7-diazaspiro[3.5]nonan-7-yl)-6-(2,2,2-trifluoroethyl) thieno[2,3-d]pyrimidine

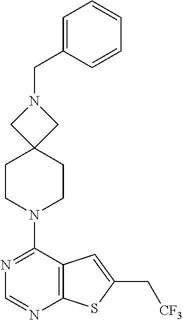 [SMILES: FC(F)(F)Cc1cc2c(N3CCC4(CC3)CN(Cc3ccccc3)C4)ncnc2s1]

### Step 1: ethyl 2-amino-5-(2,2,2-trifluoroethyl)thiophene-3-carboxylate

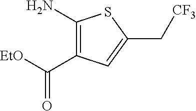 [SMILES: CCOC(=O)c1cc(CC(F)(F)F)sc1N]

To a solution of ethyl 2-cyanoacetate (35.9 g, 317.4 mmol) in anhydrous DMF (400 mL) which was cooled down to 3-5° C. under N₂ with an ice-water bath, a solution of 4,4,4-trifluorobutanal (40.0 g, 317.3 mmol) in anhydrous dimethylformamide (DMF, 20 mL) was added slowly via a syringe at 3-5° C. with stirring (the colorless clear solution slowly became an orange clear solution during addition). The addition was finished within 5 min. Then, the resulting orange mixture was stirred at 1521° C. for 10 min, after which element sulfur (10.2 g, 318.1 mmol) was added in one portion (the mixture became a dark-brown mixture quickly), and the final mixture was stirred at 1521° C. under N₂ for 24 h. LCMS and TLC (petroleum either (PE):ethyl acetate (EA)=3:1) indicated that the desired product was generated. The reaction mixture was quenched by addition of H₂O (1 L) and brine (1 L), then was extracted with EtOAc (400 mL×4). The combined organic layers were washed with brine (400 mL×2), dried over anhydrous Na₂SO₄, filtered and concentrated under reduced pressure, the residue was purified by column chromatography on silica gel eluting with petroleum ether:ethyl acetate (100:0 to 9:1) to give ethyl 2-amino-5-(2,2,2-trifluoroethyl)thiophene-3-carboxylate as a pale yellow solid. LCMS method E: Rₜ=1.081 min; (M+H)⁺=254.2. ¹H NMR (DMSO-d6): δ 7.30 (s, 2H), 6.82 (s, 1H), 4.17 (q, J=7.2 Hz, 2H), 3.67 (q, J=11.2 Hz, 1H), 1.25 (t, J=7.2 Hz, 1H).

### Step 2: 6-(2,2,2-trifluoroethyl)thieno[2,3-d]pyrimidin-4(3H)-one

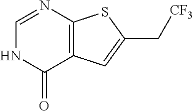 [SMILES: O=c1[nH]cnc2sc(CC(F)(F)F)cc12]

A mixture of ethyl 2-amino-5-(2,2,2-trifluoroethyl)thiophene-3-carboxylate (35.0 g, 138.2 mmol) and ammonium formate (8.7 g, 138.2 mmol) in formamide (150 mL) was heated at 135-140° C. under N₂ for 18 h. After cooling, the mixture was diluted with EtOAc (600 mL) with stirring, and the un-dissolved precipitate was removed by filtration through a pad of Celite and washed with EtOAc (100 mL×2). The combined filtrate and washings were washed with saturated aq. NaHCO₃ (300 mL), brine (300 mL×3), dried over Na₂SO₄, filtered and concentrated to give organic fraction A. All combined aqueous layers were back extracted with EtOAc (300 mL×4), then the combined organic layers were washed with brine (200 mL×2), dried over Na₂SO₄, and filtered to give organic fraction B. Organic fraction A and organic fraction B were combined, and concentrated under reduced pressure. The resulting residue was purified by column chromatography on silica gel eluting with PE:EtOAc (from 2:1 to 1:2) to give 6-(2,2,2-trifluoroethyl)thieno[2,3-d]pyrimidin-4(3H)-one (18.3 g). LCMS method E: Rₜ=0.90 min; (M+H)⁺=235.1. ¹H NMR (DMSO-d6 400 MHz): δ 12.53 (s, 1H), 8.11 (s, 1H), 7.38 (s, 1H), 4.06 (q, J=10.8 Hz, 2H).

### Step 3: 4-chloro-6-(2,2,2-trifluoroethyl)thieno[2,3-d]pyrimidine

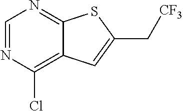 [SMILES: FC(F)(F)Cc1cc2c(Cl)ncnc2s1]

To a mixture of 6-(2,2,2-trifluoroethyl)thieno[2,3-d]pyrimidin-4(3H)-one (18.2 g, 77.7 mmol) in thionyl chloride (150 mL) was added anhydrous DMF (0.2 mL). The resulting mixture was heated at reflux temperature (7580° C. oil bath) for about 7 h (until all solid was dissolved to form a brown clear solution). After cooling down, the mixture was concentrated under reduced pressure, and the residue was purified by column chromatography on silica gel eluting with PE:EtOAc (from 100:1 to 10:1) to give 4-chloro-6-(2,2,2-trifluoroethyl)thieno[2,3-d]pyrimidine as an yellow-green oil (18.1 g); LCMS method E: Rₜ=0.780; (M+H)⁺=253.0. ¹H NMR (CDCl₃ 400 MHz): δ 8.87 (s, 1H), 7.39 (s, 1H), 3.70-3.80 (m, 2H).

### Step 4: tert-butyl 7-(6-(2,2,2-trifluoroethyl)thieno[2,3-c]pyrimidin-4-yl)-2,7-diazaspiro[3.5]nonane-2-carboxylate

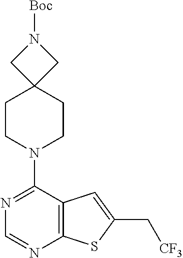 [SMILES: CC(C)(C)OC(=O)N1CC2(CCN(c3ncnc4sc(CC(F)(F)F)cc34)CC2)C1]

To a solution of tert-butyl 2,7-diazaspiro[3.5]nonane-2-carboxylate (200 mg, 0.885 mmol) in CH₃CN (5 mL) was added K₂CO₃ (366 mg, 2.655 mmol) and 4-chloro-6-(2,2,2-trifluoroethyl)thieno[2,3-d]pyrimidine (245 mg, 0.973 mmol). The reaction mixture was stirred at 60° C. for 16 h. The mixture was filtered and concentrated by rotary evaporation. The residue was purified by silica gel column (PE:EtOAc=1:1) to give compound (tert-butyl 7-(6-(2,2,2-trifluoroethyl)thieno[2,3-d]pyrimidin-4-yl)-2,7-diazaspiro[3.5]nonane-2-carboxylate) (180 mg). LCMS method C: Rₜ=0.811 min; (M+H)⁺=443.1.

### Step 5: 4-(2,7-diazaspiro[3.5]nonan-7-yl)-6-(2,2,2-trifluoroethyl)thieno[2,3-c]pyrimidine

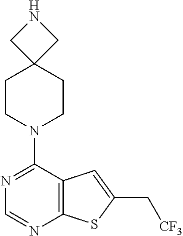 [SMILES: FC(F)(F)Cc1cc2c(N3CCC4(CC3)CNC4)ncnc2s1]

To a solution of tert-butyl 7-(6-(2,2,2-trifluoroethyl)thieno[2,3-d]pyrimidin-4-yl)-2,7-diazaspiro[3.5]nonane-2-carboxylate (180 mg, 0.41 mmol) in anhydrous DCM (5 mL) was added HCl-dioxane (4N, 1 mL). The reaction mixture was stirred at 10-12° C. for 16 h. The reaction mixture was concentrated by rotary evaporation to afford 4-(2,7-diazaspiro[3.5]nonan-7-yl)-6-(2,2,2-trifluoroethyl)thieno[2,3-d]pyrimidine (HCl salt) as a white solid, which was used for the next step directly without further purification (145 mg). LCMS method E: Rₜ=: 0.516 min;(M+H)⁺=343.2.

### Step 6: 4-(2-benzyl-2,7-diazaspiro[3.5]nonan-7-yl)-6-(2,2,2-trifluoroethyl)thieno[2,3-d]pyrimidine

To a mixture of 4-(2,7-diazaspiro[3.5]nonan-7-yl)-6-(2,2,2-trifluoroethyl)thieno[2,3-d]pyrimidine (20 mg, 0.058 mmol, HCl salt) and benzaldehyde (12 mg, 0.117 mmol) in anhydrous MeOH (2 mL) was added NaBH₃CN (14 mg, 0.232 mmol) under N₂. The mixture was stirred at 60° C. for 16 h. The mixture was concentrated under reduced pressure. The residue was purified by preparative RP-HPLC method C to give the title compound as a TFA salt (7.4 mg). LCMS method D: Rₜ=: 2.22 min; (M+H)⁺=433.1. ¹H NMR (MeOH-d4): δ 8.66 (s, 1H), 7.83 (s, 1H), 7.45-7.60 (m, 5H), 4.48 (s, 2H), 3.95-4.20 (m, 9H), 3.60-3.70 (m, 1H), 2.10-2.25 (m, 4H). ¹⁹F NMR: (MeOH-d4): δ −67.608.

### Example 2

### 4-(2-isobutyl-2,7-diazaspiro[3.5]nonan-7-yl)-6-(2,2,2-trifluoroethyl)thieno[2,3-d]pyrimidine

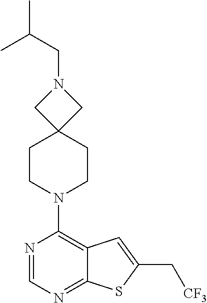 [SMILES: CC(C)CN1CC2(CCN(c3ncnc4sc(CC(F)(F)F)cc34)CC2)C1]

The title compound was prepared using procedures analogous to those described in Example 1. In step 6, isobutraldehyde was used instead of benzaldehyde. The title compound was isolated as a TFA salt. LCMS method C: Rₜ=: 0.630 min; (M+H)⁺=399.2. ¹H NMR (CD₃OD): δ 8.44 (s, 1H), 7.58 (s, 1H), 4.23 (d, J=11.6 Hz, 2H), 3.85-4.02 (m, 8H), 3.15 (d, J=7.2 Hz, 2H), 1.93-2.12 (m, 5H), 1.02 (d, J=7.2 Hz, 3H). ¹⁹F NMR: (CD₃OD): δ −67.676, −77.243.

### Example 3

### 4-(2-isopropyl-2,7-diazaspiro[3.5]nonan-7-yl)-6-(2,2,2-trifluoroethyl)thieno[2,3-d]pyrimidine

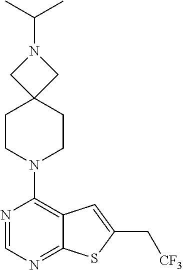 [SMILES: CC(C)N1CC2(CCN(c3ncnc4sc(CC(F)(F)F)cc34)CC2)C1]

The title compound was prepared using procedures analogous to those described in Example 1. In step 6, acetone was used instead of benzaldehyde. The title compound was isolated as a TFA salt. LCMS method C: Rₜ=: 0.622 min; (M+H)⁺=385.2. ¹H NMR (CD₃OD): δ 8.43 (s, 1H), 7.56 (s, 1H), 4.12 (d, J=11.2 Hz, 2H), 3.90-4.05 (m, 8H), 3.45-3.55 (m, 1H), 1.97-2.12 (m, 4H), 1.29 (d, J=6.4 Hz, 3H). ¹⁹F NMR (CD₃OD): δ −67.684, −77.000.

### Example 4

### 4-(2-((tetrahydrofuran-2-yl)methyl)-2,7-diazaspiro[3.5]nonan-7-yl)-6-(2,2,2-trifluoroethyl)thieno[2,3-d]pyrimidine

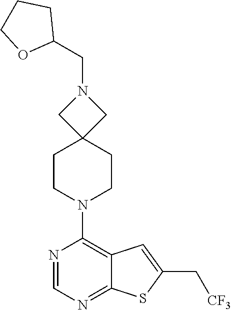 [SMILES: FC(F)(F)Cc1cc2c(N3CCC4(CC3)CN(CC3CCCO3)C4)ncnc2s1]

The title compound was prepared using procedures analogous to those described in Example 1. In step 6, tetrahydrofuran-2-carbaldehyde (CAS registry number 7681-84-7) was used instead of benzaldehyde. LCMS method D: Rₜ=1.93 min; (M+H)⁺=427.1. ¹H NMR (CD₃OD): δ 8.35 (s, 1H), 7.53 (s, 1H), 3.73-3.95 (m, 9H), 3.29 (s, 4H), 2.64-2.60 (m, 2H), 1.84-2.03 (m, 7H), 1.52-1.58 (m, 1H). ¹⁹F NMR (CD₃OD): δ −67.691.

### Example 5

### 4-(2-((tetrahydrofuran-3-yl)methyl)-2,7-diazaspiro[3.5]nonan-7-yl)-6-(2,2,2-trifluoroethyl)thieno[2,3-d]pyrimidine

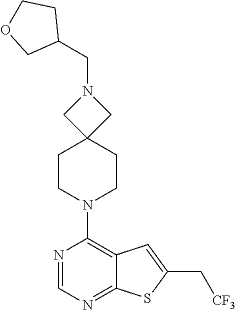 [SMILES: FC(F)(F)Cc1cc2c(N3CCC4(CC3)CN(CC3CCOC3)C4)ncnc2s1]

The title compound was prepared using procedures analogous to those described in Example 1. In step 6, tetrahydrofuran-3-carbaldehyde (CAS registry number 79710-86-4) was used instead of benzaldehyde. LCMS method D: Rₜ=1.80 min; (M+H)⁺=427.1. ¹H NMR (CD₃OD): δ 8.35 (s, 1H), 7.53 (s, 1H), 3.70-3.93 (m, 9H), 3.40-3.50 (m, 1H), 3.22 (s, 4H), 2.55-2.65 (m, 2H), 2.30-2.40 (m, 1H), 2.00-2.10 (m. 1H), 1.90-1.95 (m, 4H), 1.55-1.65 (m, 1H). ¹⁹F NMR (CD₃OD): δ −67.668.

### Example 6

### 4-(2-((tetrahydro-2H-pyran-2-yl)methyl)-2,7-diazaspiro[3.5]nonan-7-yl)-6-(2,2,2-trifluoroethyl)thieno[2,3-d]pyrimidine

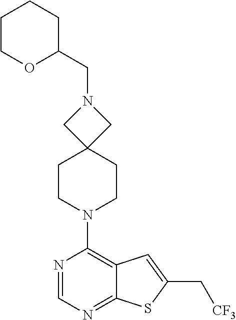 [SMILES: FC(F)(F)Cc1cc2c(N3CCC4(CC3)CN(CC3CCCCO3)C4)ncnc2s1]

The title compound was prepared using procedures analogous to those described in Example 1. In step 6, tetrahydro-2H-pyran-2-carbaldehyde (CAS registry number 19611-45-1) was used instead of benzaldehyde. LCMS method D: Rₜ=2.06 min; (M+H)⁺=440.9. ¹H NMR (CD₃OD): δ 8.35 (s, 1H), 7.53 (s, 1H), 3.84-3.96 (m, 7H), 3.39-3.44 (m, 2H), 3.26 (s, 4H), 2.55-2.69 (m, 2H), 1.84-1.93 (m, 5H), 1.53-1.60 (m, 4H), 1.23-1.29 (m, 1H). ¹⁹F NMR (CD₃OD): δ −67.676.

### Example 7

### 4-(2-((tetrahydro-2H-pyran-3-yl)methyl)-2,7-diazaspiro[3.5]nonan-7-yl)-6-(2,2,2-trifluoroethyl)thieno[2,3-d]pyrimidine

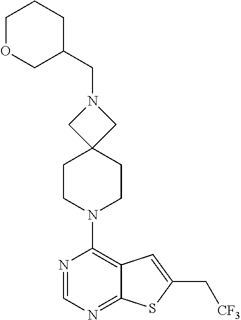 [SMILES: FC(F)(F)Cc1cc2c(N3CCC4(CC3)CN(CC3CCCOC3)C4)ncnc2s1]

The title compound was prepared using procedures analogous to those described in Example 1. In step 6, tetrahydro-2H-pyran-3-carbaldehyde (CAS registry number 77342-93-9) was used instead of benzaldehyde. The title compound was isolated as a TFA salt. LCMS method C: Rₜ=0.60 min; (M+H)⁺=440.9. ¹H NMR (CD₃OD): δ 8.43 (s, 1H), 7.57 (s, 1H), 4.15-4.25 (m, 2H), 3.85-4.00 (m, 8H), 3.60-3.80 (m, 2H), 3.35-3.45 (m, 1H), 3.10-3.20 (m, 3H), 1.85-2.10 (m, 6H), 1.50-1.65 (m, 2H), 1.30-1.40 (m, 1H). ¹⁹F NMR (CD₃OD): δ −67.641, 77.202.

### Example 8

### 4-(2-(tetrahydro-2H-pyran-4-yl)-2,7-diazaspiro[3.5]nonan-7-yl)-6-(2,2,2-trifluoroethyl)thieno[2,3-d]pyrimidine

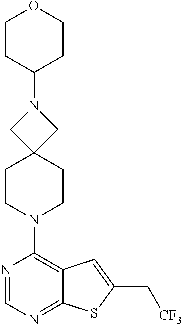 [SMILES: FC(F)(F)Cc1cc2c(N3CCC4(CC3)CN(C3CCOCC3)C4)ncnc2s1]

The title compound was prepared using procedures analogous to those described in Example 1. In step 6, tetrahydro-2H-pyran-4-none (CAS registry number 143562-54-3) was used instead of benzaldehyde. The title compound was isolated as a TFA salt. LCMS method C: Rₜ=0.59 min; (M+H)⁺=426.9. ¹H NMR (CD₃OD): δ 8.40 (s, 1H), 7.55 (s, 1H), 3.95-4.15 (m, 8H), 3.85-3.95 (m, 4H), 3.30-3.50 (m, 3H), 1.90-2.10 (m, 6H), 1.40-1.50 (m, 2H). ¹⁹F NMR (CD₃OD): δ −67.677, −77.082.

### Example 9

### 4-(2-cyclohexyl-2,7-diazaspiro[3.5]nonan-7-yl)-6-(2,2,2-trifluoroethyl)thieno[2,3-d]pyrimidine

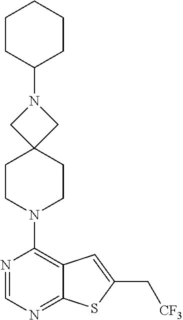 [SMILES: FC(F)(F)Cc1cc2c(N3CCC4(CC3)CN(C3CCCCC3)C4)ncnc2s1]

The title compound was prepared using procedures analogous to those described in Example 1. In step 6, cyclohexanone was used instead of benzaldehyde. LCMS method C: Rₜ=0.66 min; (M+H)⁺=424.9. ¹H NMR (CD₃OD): δ 8.35 (s, 1H), 7.53 (s, 1H), 3.85-3.98 (m, 6H), 3.20 (s, 4H), 2.10-2.20 (m, 1H), 1.65-1.95 (m, 9H), 1.10-1.35 (m. 3H), 0.95-1.05 (m, 2H). ¹⁹F NMR (CD₃OD): δ −67.678.

### Example 10

### 4-(2-(5,5-dimethyl-4,5-dihydrothiazol-2-yl)-2,7-diazaspiro[3.5]nonan-7-yl)-6-(2,2,2-trifluoroethyl)thieno[2,3-d]pyrimidine

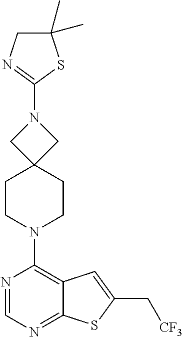 [SMILES: CC1(C)CN=C(N2CC3(CCN(c4ncnc5sc(CC(F)(F)F)cc45)CC3)C2)S1]

### Step 1: N-(2-methylallyl)-7-(6-(2,2,2-trifluoroethyl)thieno[2,3-d]pyrimidin-4-yl)-2,7-diazaspiro[3.5] nonane-2-carbothioamide

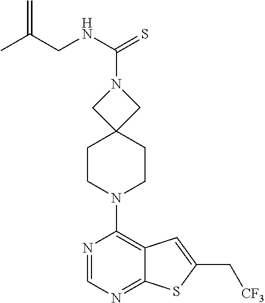 [SMILES: C=C(C)CNC(=S)N1CC2(CCN(c3ncnc4sc(CC(F)(F)F)cc34)CC2)C1]

To a solution of 4-(2,7-diazaspiro[3.5]nonan-7-yl)-6-(2,2,2-trifluoroethyl)thieno[2,3-d]pyrimidine (50 mg, 0.146 mmol) in anhydrous DCM (3 mL) was added TEA (147 mg, 0.2 mL, 1.46 mmol) and 3-isothiocyanato-2-methylprop-1-ene (20 mg, 0.175 mmol). The reaction mixture was stirred at 15˜18° C. for 2 h. The reaction mixture was concentrated by rotary evaporation, and the residue was purified by preparative TLC (PE:EtOAc=1:1) to give N-(2-methylallyl)-7-(6-(2,2,2-trifluoroethyl)thieno[2,3-d]pyrimidin-4-yl)-2,7-diazaspiro[3.5] nonane-2-carbothioamide (30 mg). LCMS method E: Rₜ=1.16 min; (M+H)⁺=456.2.

### Step 2: (4-(2-(5,5-dimethyl-4,5-dihydrothiazol-2-yl)-2,7-diazaspiro[3.5] nonan-7-yl)-6-(2,2,2-trifluoroethypthieno[2,3-d]pyrimidine)

A solution of N-(2-methylallyl)-7-(6-(2,2,2-trifluoroethyl)thieno[2,3-d]pyrimidin-4-yl)-2,7-diazaspiro[3.5]nonane-2-carbothioamide (30 mg, 0.066 mmol) in AcOH (3 mL) was stirred at 90° C. for 16 h. The mixture was concentrated under reduced pressure. The residue was purified by preparative RP-HPLC method B to give (4-(2-(5,5-dimethyl-4,5-dihydrothiazol-2-yl)-2,7-diazaspiro[3.5]nonan-7-yl)-6-(2,2,2-trifluoroethyl)thieno[2,3-d]pyrimidine) as a white solid (21.0 mg). LCMS method C: Rₜ=0.69 min; (M+H)⁺=456.1; ¹H NMR: (MeOH-d4 400 MHz): δ 8.44 (s, 1H), 7.58 (s, 1H), 4.16 (d, J=13.2 Hz, 4H), 3.85-4.18 (m, 8H), 2.04-2.07 (m, 4H), 1.70 (s, 3H); ¹⁹F NMR: (MeOH-d4 400 MHz): δ −67.676, −77.137.

### Example 11

### 4-(2-((3,3-difluorocyclohexyl)methyl)-2,7-diazaspiro[3.5]nonan-7-yl)-6-(2,2,2-trifluoroethyl)thieno[2,3-d]pyrimidine

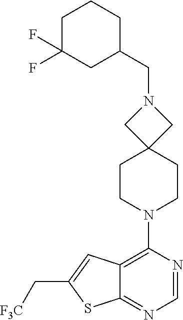 [SMILES: FC(F)(F)Cc1cc2c(N3CCC4(CC3)CN(CC3CCCC(F)(F)C3)C4)ncnc2s1]

### Step 1: 3,3-difluorocyclohexanecarbaldehyde

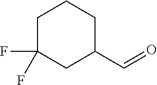

A mixture of (3,3-difluorocyclohexyl)methanol (100 mg, 0.67 mmol) and PCC (289 mg, 1.34 mmol) in anhydrous DCM (5 mL) was stirred at 15-23° C. for 18 h. TLC (petroleum ether:ethyl acetate=3:1) showed the reaction was completed. The mixture was filtered and the filtrate was concentrated under reduced pressure. The residue was purified by column chromatograph on silica gel (eluting with petroleum ether:ethyl acetate=3:1 to 2:1) to give 3,3-difluorocyclohexanecarbaldehyde (60 mg, 61%) as a colourless oil. ¹H NMR (CDCl₃): δ 9.61 (s, 1H), 2.54-2.56 (m, 1H), 2.25-2.27 (m, 8H).

### Step2: 4-(2-((3,3-difluorocyclohexyl)methyl)-2,7-diazaspiro[3.5]nonan-7-yl)-6-(2,2,2-trifluoroethyl)thieno[2,3-d]pyrimidine

A mixture of 4-(2,7-diazaspiro[3.5]nonan-7-yl)-6-(2,2,2-trifluoroethyl)thieno[2,3-d]pyrimidine (100 mg, 0.088 mmol), 3,3-difluorocyclohexanecarbaldehyde (39 mg, 0.26 mmol), NaBH₃CN (28 mg, 0.44 mmol) and HOAc (3 drops) in MeOH (3 mL) was stirred at 15-24° C. for 18 h. The mixture was concentrated under reduced pressure and the residue was purified by preparative RP-HPLC method B and then dried by lyophilization to give the title compound (TFA salt) as a yellow solid (14 mg). LCMS method C: Rₜ=0.91 min; (M+H)⁺=475.2; ¹H NMR (MeOH-d4): δ 8.48 (s, 1H), 7.61 (s, 1H), 4.25-4.31 (m, 2H), 3.93-4.08 (m, 8H), 3.26-3.31 (m, 2H), 1.53-2.15 (m, 12H), 1.13-1.20 (m, 1H); ¹⁹F NMR (MeOH-d4): δ −67.66, −77.21, −90.73, −90.09.

### Example 12

### 4-(2((4,4-difluorocyclohexyl)methyl)-2,7-diazaspiro[3.5] nonan-7-yl)-6-(2,2,2-trifluoroethyl)thieno[2,3-d]pyrimidine

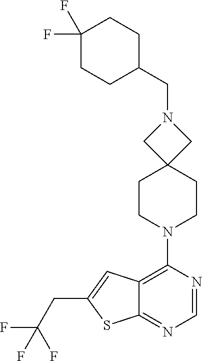 [SMILES: FC(F)(F)Cc1cc2c(N3CCC4(CC3)CN(CC3CCC(F)(F)CC3)C4)ncnc2s1]

The title compound was prepared using procedures analogous to those described in Example 11. In step 2, 3,3-difluorocyclohexanecarbaldehyde was used. The title compound was isolated as an HCl salt (purified by preparative RP-HPLC method C). LCMS method C: Rₜ=0.77 min; (M+H)⁺=475.1. ¹H NMR (Methanol-d4): δ 8.67 (s, 1H), 7.85 (s, 1H), 4.31 (d, J=10.8 Hz, 2H), 4.22 (m, 2H), 4.10 (m, 6H), 3.28 (d, J=6.5 Hz, 2H), 2.28 (m, 2H), 2.11 (m, 4H), 1.84 (m, 5H), 1.38 (m, 2H). ¹⁹F NMR (Methanol-d4): δ −67.558˜67.613, −93.201˜93.827, −103.398˜104.204.

### Example 13

### 4-(2-((3-methoxycyclobutyl)methyl)-2,7-diazaspiro[3.51nonan-7-yl)-6-(2,2,2-trifluoroethyl)thieno[2,3-d]pyrimidine

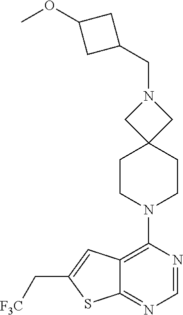 [SMILES: COC1CC(CN2CC3(CCN(c4ncnc5sc(CC(F)(F)F)cc45)CC3)C2)C1]

### Step 1: (3-methoxycyclobutyl)(7-(6-(2,2,2-trifluoroethyl)thieno[2,3-d]pyrimidin-4-yl)-2,7-diazaspiro[3.5]nonan-2-yl)methanone

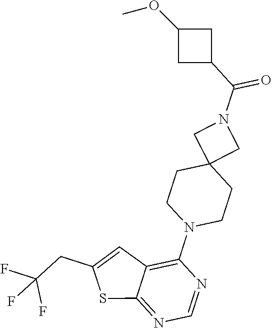 [SMILES: COC1CC(C(=O)N2CC3(CCN(c4ncnc5sc(CC(F)(F)F)cc45)CC3)C2)C1]

A mixture of 4-(2,7-diazaspiro[3.5]nonan-7-yl)-6-(2,2,2-trifluoroethyl)thieno[2,3-d]pyrimidine (300 mg, 0.88 mmol), 3-methoxycyclobutanecarboxylic acid (CAS registry number 480450-03-1; 126 mg, 0.97 mmol), HATU (502 mg, 1.32 mmol) and Et₃N (445 mg, 0.61 mL, 4.4 mmol) in anhydrous DMF (10 mL) was stirred at 20-23° C. for 3 h. The mixture was added to H₂O (20 mL) and extracted with EtOAc (3×30 mL). The combined organic layers were washed with H₂O (3×20 mL) and brine (20 mL), dried over anhydrous sodium sulfate, filtered and concentrated under reduced pressure. The residue was purified by column chromatograph on silica gel (eluting with CH₂Cl₂:MeOH=8:1 to 7:1) to give (3-methoxycyclobutyl)(7-(6-(2,2,2-trifluoroethyl)thieno[2,3-d]pyrimidin-4-yl)-2,7-diazaspiro[3.5]nonan-2-yl)methanone (100 mg); LCMS method C: Rₜ=0.67 min; (M+H)⁺=454.9. ¹H NMR (MeOH-d4): δ 8.49 (s, 1H), 7.65 (s, 1H), 3.80-4.06 (m, 11H), 3.25 (s, 3H), 2.71-2.74 (m, 1H), 2.43-2.49 (m, 2H), 2.09-2.13 (m, 2H), 1.96-1.99 (m, 4H). ¹⁹F NMR (MeOH-d4): δ −67.67, −77.38.

### Step 2: 4-(2-((3-methoxycyclobutyl)methyl)-2,7-diazaspiro[3.5]nonan-7-yl)-6-(2,2,2-trifluoroethyl)thieno[2,3-d]pyrimidine

To a mixture of (3-methoxycyclobutyl)(7-(6-(2,2,2-trifluoroethyl)thieno[2,3-d]pyrimidin-4-yl)-2,7-diazaspiro[3.5]nonan-2-yl)methanone (45 mg, 0.1 mmol) in anhydrous THF (2 mL) was added BH₃/Me₂S (0.2 mL, 2 mmol, 10 M in Me₂S) dropwise under ice-water. The mixture was stirred at 21-24° C. for 18 h. MeOH (3 mL) was added dropwise to quench the mixture and the mixture was concentrated under reduced pressure. The residue was dissolved in MeOH (5 mL) and stirred at reflux for 2 h. The mixture was concentrated under reduced pressure and the residue was purified by acidic preparative RP-HPLC method A to give the title compound (2.2 mg). LCMS method C: Rₜ=0.64 min; (M+H)⁺=441.2; ¹H NMR (MeOH-d4): δ 8.40 (s, 1H), 7.53 (s, 1H), 4.15-4.19 (m, 2H), 3.85-3.99 (m, 10H), 3.30-3.35 (m, 2H), 3.26 (s, 3H), 2.48-2.53 (m, 1H), 2.00-2.16 (m, 6H), 1.68-1.75 (m, 2H); ¹⁹F NMR (MeOH-d4): δ −67.67, −76.96.

### Example 14

### 4-(2-((3,3-difluorocyclobutyl)methyl)-2,7-diazaspiro[3.5]nonan-7-yl)-6-(2,2,2-trifluoroethyl)thieno[2,3-d]pyrimidine

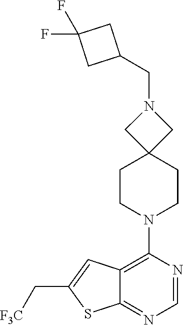 [SMILES: FC(F)(F)Cc1cc2c(N3CCC4(CC3)CN(CC3CC(F)(F)C3)C4)ncnc2s1]

### Step 1: (3,3-difluorocyclobutyl)methyl-4-methylbenzenesulfonate

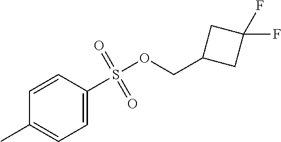 [SMILES: Cc1ccc(S(=O)(=O)OCC2CC(F)(F)C2)cc1]

To a solution of (3,3-difluorocyclobutyl)methanol (CAS registry number 681128-39-2; 200 mg, 1.64 mmol) in anhydrous CH₂Cl₂ (5 mL) was added TsCl (312 mg, 1.64 mmol) and Et₃N (828 mg, 8.2 mmol). The reaction was stirred at 19-28° C. for 16 h. The mixture was concentrated under reduced pressure. The residue was purified by chromatography on silica gel (PE:EtOAc=5:1) to afford (3,3-difluorocyclobutyl)methyl 4-methylbenzenesulfonate (320 mg). ¹H NMR: (CDCl₃): δ 7.80 (d, J=8.4 Hz, 2H), 7.36 (d, J=8.4 Hz, 2H), 4.06 (d, J=6.8 Hz, 2H), 2.63 (m, 2H), 5.50 (m, 1H), 2.46 (s, 3H), 2.29 (m, 2H).

### Step 2: 4-(2-((3,3-difluorocyclobutyl)methyl)-2,7-diazaspiro[3.5]nonan-7-yl)-6-(2,2,2-trifluoroethyl)thieno[2,3-4pyrimidine

To a mixture of 4-(2,7-diazaspiro[3.5]nonan-7-yl)-6-(2,2,2-trifluoroethyl)thieno[2,3-d]pyrimidine (50 mg, 0.146 mol) in anhydrous DMF (5 mL) was added (3,3-difluorocyclobutyl)methyl-4-methylbenzenesulfonate (81 mg, 0.292 mmol) and Et₃N (74 mg, 0.73 mmol). The reaction was stirred at 100° C. for 16 h under N₂ atmosphere. The mixture was diluted with water (10 mL) and extracted with EtOAc (3×10 mL). The combined organic layers were washed with water (3×20 mL), dried over anhydrous Na₂SO₄, filtered and concentrated under reduced pressure. The residue was purified by acidic preparative RP-HPLC method C and the solution was lyophilized to afford the title compound. LCMS method C: Rₜ=0.63 min; (M+H)⁺=447.0; ¹H NMR (MeOH-d4): δ 8.63 (s, 1H), 7.79 (s, 1H), 4.17-4.25 (m, 4H), 4.02-4.10 (m, 6H), 3.47-3.48 (d, J=6.4 Hz, 2H), 2.76-2.83 (m, 2H), 2.44-2.55 (m, 3H), 2.21-2.24 (m, 2H), 2.09-2.12 (m, 2H); ¹⁹F NMR (MeOH-d4): δ −67.67, −84.59, −97.91.

### Example 15

### 2-methyl-1-(7-(6-(2,2,2-trifluoroethyl)thieno[2,3-d]pyrimidin-4-yl)-2,7-diazaspiro[3.5]nonan-2-yl)propan-2-ol

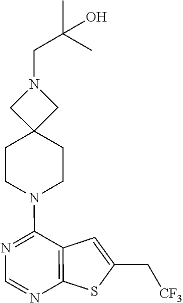 [SMILES: CC(C)(O)CN1CC2(CCN(c3ncnc4sc(CC(F)(F)F)cc34)CC2)C1]

A solution of 4-(2,7-diazaspiro[3.5]nonan-7-yl)-6-(2,2,2-trifluoroethyl)thieno[2,3-d]pyrimidine hydrochloride (50 mg, 0.13 mmol), 2,2-dimethyloxirane (48 mg, 0.65 mmol) and TEA (60 mg, 0.65 mmol) in EtOH (3 mL) was stirred at 60° C. for 16 h under N₂. The reaction mixture was concentrated in vacuo and purified by preparative TLC on silica gel (MeOH:CH₂Cl₂=1:20) to give the title compound (30 mg). LCMS method C: Rₜ=0.60 min; (M+H)⁺=415.2; ¹H NMR (MeOH-d4): δ 8.36 (s, 1H), 7.52 (s, 1H), 3.68-4.05 (m, 10H), 2.81-3.24 (m, 2H), 1.94-2.10 (m, 4H), 1.25 (s, 6H); ¹⁹F NMR (MeOH-d4): δ −67.684.

### Example 16

### 4-(2-(2-fluoro-2-methylpropyl)-2,7-diazaspiro[3.5]nonan-7-yl)-6-(2,2,2-trifluoroethyl)thieno[2,3-d]pyrimidine

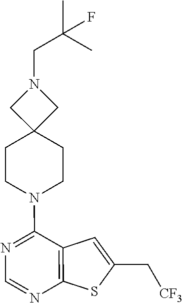 [SMILES: CC(C)(F)CN1CC2(CCN(c3ncnc4sc(CC(F)(F)F)cc34)CC2)C1]

To a solution of 2-methyl-1-(7-(6-(2,2,2-trifluoroethyl)thieno[2,3-d]pyrimidin-4-yl)-2,7-diazaspiro[3.5]nonan-2-yl)propan-2-ol (30 mg, 0.07 mmol) in CH₂Cl₂ (5 mL, anhydrous) was added diethylaminosulfur trifluoride (DAST) (112 mg, 0.07 mmol) dropwise at −78° C. under N₂. The resulting mixture was stirred at −78° C. for 2 h under N₂. The reaction mixture was quenched with water (5 mL), and extracted with CH₂Cl₂ (3×5 mL). The organic layers were washed with brine (5 mL), dried over Na₂SO₄, filtered, concentrated in vacuo, purified by acidic preparative RP-HPLC method A and dried by lyophilization to give the title compound (7.0 mg). LCMS method C: Rₜ=0.65 min; (M+H)⁺=417.1; ¹H NMR (MeOH-d4): δ 8.40 (s, 1H), 7.53 (s, 1H), 4.32 (s, 2H), 4.15 (s, 2H), 3.85-4.00 (m, 6H), 3.62 (d, J=19.6 Hz, 2H), 1.98-2.16 (m, 4H), 1.51 (s, 3H), 1.46 (s, 3H); ¹⁹F NMR (MetOH-d4): δ −67.656, −77.049, −144.911.

### Example 17

### 3-amino-3-methyl-1-(7-(6-(2,2,2-trifluoroethyl)thieno[2,3-d]pyrimidin-4-yl)-2,7-diazaspiro[3.5]nonan-2-yl)butan-1-one

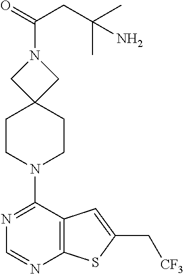 [SMILES: CC(C)(N)CC(=O)N1CC2(CCN(c3ncnc4sc(CC(F)(F)F)cc34)CC2)C1]

### Step 1: tert-butyl (2-methyl-4-oxo-4-(7-(6-(2,2,2-trifluoroethyl)thieno[2,3-dipyrimidin-4-yl)-2,7-diazaspiro[3.5]nonan-2-yl)butan-2-yl)carbamate

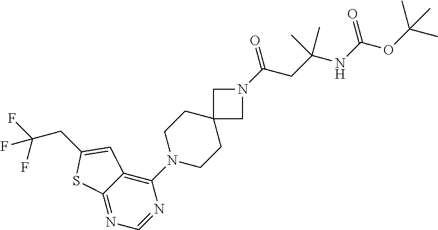 [SMILES: CC(C)(CC(=O)N1CC2(CCN(c3ncnc4sc(CC(F)(F)F)cc34)CC2)C1)NC(=O)OC(C)(C)C]

A solution of 3-((tert-butoxycarbonyl)amino)-3-methylbutanoic acid (CAS registry number 129765-95-3; 29 mg, 0.13 mmol), HATU (50 mg, 0.13 mmol) and DIEA (70 mg, 0.52 mmol) in DMF (5 mL, anhydrous) was stirred at 21-27° C. for 20 min, to which 4-(2,7-diazaspiro[3.5]nonan-7-yl)-6-(2,2,2-trifluoroethyl)thieno[2,3-d]pyrimidine hydrochloride (50 mg, 0.13 mmol) was added. The resulting mixture was stirred at 21-27° C. for about 3 h under N₂. The resulting mixture was quenched with water (30 mL) and extracted with EtOAc (2×20 mL). The organic layers were washed with brine (20 mL), filtered and concentrated to give tert-butyl (2-methyl-4-oxo-4-(7-(6-(2,2,2-trifluoroethyl)thieno[2,3-d]pyrimidin-4-yl)-2,7-diazaspiro[3.5]nonan-2-yl)butan-2-yl)carbamate (140 mg, crude) as a yellow oil, which was used for next step directly without further purification.

### Step 2: 3-amino-3-methyl-1-(7-(6-(2,2,2-trifluoroethyl)thieno[2,3-d]pyrimidin-4-yl)-2,7-diazaspiro[3.5]nonan-2-yl)butan-1-one

A solution of tert-butyl (2-methyl-4-oxo-4-(7-(6-(2,2,2-trifluoroethyl)thieno[2,3-d]pyrimidin-4-yl)-2,7-diazaspiro[3.5]nonan-2-yl)butan-2-yl)carbamate (140 mg, 0.13 mmol, crude) in a mixture of TFA:CH₂Cl₂ mL, V:V=1:3) was stirred at 21-24° C. for about 2 h. The resulting mixture was concentrated in vacuo and purified by acidic preparative RP-HPLC Method B and dried by lyophilization to give the title compound (11 mg). LCMS method C: Rₜ=0.64 min; (M+H)⁺=442.2; ¹H NMR (MeOH-d4): δ 8.44 (s, 1H), 7.60 (s, 1H), 4.06 (s, 2H), 3.89-4.02 (m, 6H), 3.85 (s, 2H), 2.51 (s, 2H), 1.90-2.04 (m, 4H), 1.41 (s, 6H); ¹⁹F NMR (MeOH-d4): δ −67.653.

### Example 18

### 4-(2-neopentyl-2,7-diazaspiro[3.5]nonan-7-yl)-6-(2,2,2-trifluoroethyl)thieno[2,3-d]pyrimidine

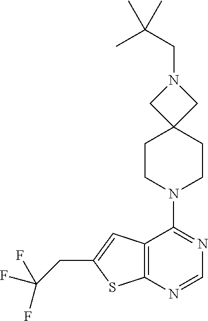 [SMILES: CC(C)(C)CN1CC2(CCN(c3ncnc4sc(CC(F)(F)F)cc34)CC2)C1]

The title compound was prepared using procedures analogous to those described in Example 1. In step 6, 3,3-dimethylbutanal was used instead of benzaldehyde. The title compound was isolated as a TFA salt. LCMS method B: Rₜ=0.93 min; (M+H)⁺=413.4; ¹H NMR (MeOH-d₄): δ (ppm) 8.44 (s, 1H), 7.60 (s, 1H), 4.35 (d, J=16 Hz, 2H), 4.06 (d, J=16 Hz, 2H), 4.00 (m, 2H), 3.92 (m, 4H), 3.22 (s, 2H), 2.16 (m, 2H), 2.04 (m, 2H), 1.04 (s, 9H). ¹⁹F NMR (MeOH-d₄): δ −67.687.

### Example 19

### 4-(2-((tetrahydro-2H-pyran-4-yl)methyl)-2,7-diazaspiro[3.5]nonan-7-yl)-6-(2,2,2-trifluoroethyl)thieno[2,3-d]pyrimidine

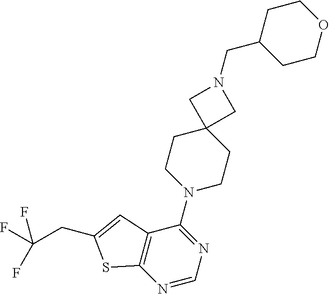 [SMILES: FC(F)(F)Cc1cc2c(N3CCC4(CC3)CN(CC3CCOCC3)C4)ncnc2s1]

The title compound was prepared using procedures analogous to those described in Example 1. In step 6, 4-tetrahydropyrane carbaldehyde (CAS registry number 50675-18-8) was used instead of benzaldehyde. The title compound was isolated as a TFA salt. LCMS method B: Rₜ=0.84 min; (M+H)⁺=441.4.

### Example 20

### 3-((7-(6-(2,2,2-trifluoroethyl)thieno[2,3-d]pyrimidin-4-yl)-2,7-diazaspiro[3.5]nonan-2-yl)methyl)-1H-indole-6-carbonitrile

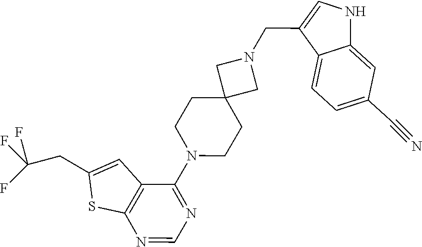 [SMILES: N#Cc1ccc2c(CN3CC4(CCN(c5ncnc6sc(CC(F)(F)F)cc56)CC4)C3)c[nH]c2c1]

The title compound was prepared using procedures analogous to those described in Example 1. In step 6, 3-formyl-1H-indole-6-carbonitrile was used instead of benzaldehyde. The title compound was isolated as a TFA salt. LCMS method B: Rₜ=0.98 min; (M+H)⁺=497.4; ¹H NMR (MeOH-d4): δ 8.44 (s, 1H), 7.94 (d, J=4 Hz, 1H), 7.82 (2 singlet, 2H), 7.60 (s, 1H), 7.40 (d, J=4 Hz, 1H), 4.64 (s, 2H), 3.92-4.12 (m, 10H), 2.04 (m, 4H). ¹⁹F NMR (MeOH-d4): δ −67.669.

### Example 21

### 4-methyl-5-((7-(6-(2,2,2-trifluoroethyl)thieno[2,3-d]pyrimidin-4-yl)-2,7-diazaspiro[3.5]nonan-2-yl)methyl)-1H-indole-2-carbonitrile

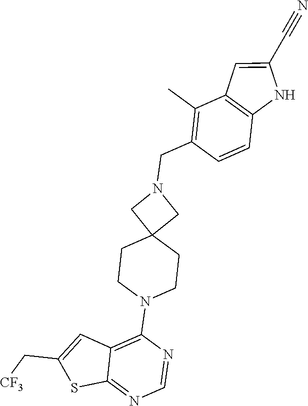 [SMILES: Cc1c(CN2CC3(CCN(c4ncnc5sc(CC(F)(F)F)cc45)CC3)C2)ccc2[nH]c(C#N)cc12]

The title compound was prepared using procedures analogous to those described in Example 1. In step 6, 5-formyl-4-methyl-1H-indole-2-carbonitrile was used instead of benzaldehyde. 5-Formyl-4-methyl-1H-indole-2-carbonitrile was synthesized by the method described in Borkin et al., Cancer Cell, 27, 1-14, 2015. The title compound was isolated as a TFA salt. LCMS method B: Rₜ=1.04 min; (M+H)⁺=511.5.

### Example 22

### 4-(2-(2-methylbutyl)-2,7-diazaspiro[3.5]nonan-7-yl)-6-(2,2,2-trifluoroethyl)thieno[2,3-d]pyrimidine

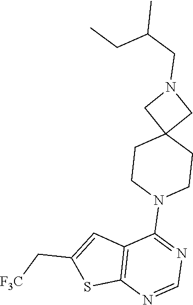 [SMILES: CCC(C)CN1CC2(CCN(c3ncnc4sc(CC(F)(F)F)cc34)CC2)C1]

The title compound was prepared using procedures analogous to those described in Example 1. In step 6, 2-methylbutanal was used instead of benzaldehyde. The title compound was isolated as a TFA salt. LCMS method B: Rₜ=0.95 min; (M+H)⁺=413.4; ¹H NMR (methanol-d₄): δ 8.44 (s, 1H), 7.60 (s, 1H), 4.24 (m, 2H), 3.98 (m, 8H), 3.24 (m, 1H), 3.12 (m, 1H), 2.02-2.12 (m, 4H), 1.78 (m, 1H), 1.46 (m, 1H), 1.24 (m, 1H), 0.96 (m, 6H). ¹⁹F NMR (methanol-d₄): δ −67.673.

### Example 23

### 4-(2-benzyl-2,8-diazaspiro[4.5]decan-8-yl)-6-(2,2,2-trifluoroethyl)thieno[2,3-d]pyrimidine

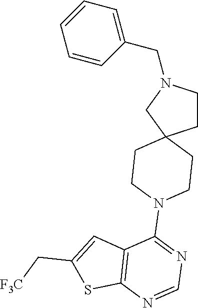 [SMILES: FC(F)(F)Cc1cc2c(N3CCC4(CCN(Cc5ccccc5)C4)CC3)ncnc2s1]

### Step 1. tert-butyl 8-(6-(2,2,2-trifluoroethyl)thieno[2,3-d]pyrimidin-4-yl)-2,8-diazaspiro[4.5]decane-2-carboxylate

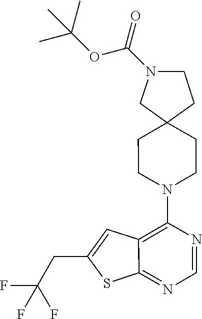 [SMILES: CC(C)(C)OC(=O)N1CCC2(CCN(c3ncnc4sc(CC(F)(F)F)cc34)CC2)C1]

To a solution of 4-chloro-6-(2,2,2-trifluoroethyl)thieno[2,3-d]pyrimidine (120 mg, 0.476 mmol) and tert-butyl 2,8-diazaspiro[4.5]decane-2-carboxylate (CAS registry number 336191-17-4; 171 mg, 0.714 mmol) in CH₃CN (3 mL) was added K₂CO₃ (197 mg, 1.428 mmol) under N₂ atmosphere. The mixture was stirred at 70° C. for 2 h. LCMS showed 4-chloro-6-(2,2,2-trifluoroethyl)thieno[2,3-d]pyrimidine was consumed completely. The mixture was filtered and concentrated under reduced pressure. The residue was purified by column chromatography on silica gel (Petroleum ether:Ethyl acetate=1:1) to afford tert-butyl 8-(6-(2,2,2-trifluoroethyl)thieno[2,3-d]pyrimidin-4-yl)-2,8-diazaspiro[4.5]decane-2-carboxylate as a light yellow oil (200 mg, 69%); LCMS method C: Rₜ=0.84 min; (M+H)⁺=457.2.

### Step 2. 4-(2,8-diazaspiro[4.5]decan-8-yl)-6-(2,2,2-trifluoroethyl)thieno[2,3-d]pyrimidine

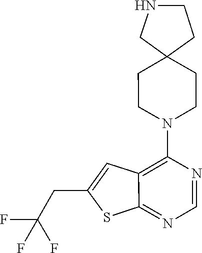 [SMILES: FC(F)(F)Cc1cc2c(N3CCC4(CCNC4)CC3)ncnc2s1]

To a solution of tert-butyl 8-(6-(2,2,2-trifluoroethyl)thieno[2,3-d]pyrimidin-4-yl)-2,8-diazaspiro[4.5]decane-2-carboxylate (200 mg, 0.438 mmol) in anhydrous CH₂Cl₂ (5 mL) was added HCl-dioxane (4 N, 2 mL) at 10-12° C. The mixture was stirred at 10-12° C. for 16 h. LCMS showed tert-butyl 8-(6-(2,2,2-trifluoroethyl)thieno[2,3-d]pyrimidin-4-yl)-2,8-diazaspiro[4.5]decane-2-carboxylate was consumed completely. The mixture was concentrated under reduced pressure to afford crude 4-(2,8-diazaspiro[4.5]decan-8-yl)-6-(2,2,2-trifluoroethyl)thieno[2,3-d]pyrimidine (HCl salt) as a gray-white solid (156 mg,100%), which was used for next step without further purification. LCMS method D: Rₜ=0.91 min; (M+H)⁺=357.2.

### Step 3. (4-(2-benzyl-2,8-diazaspiro[4.5]decan-8-yl)-6-(2,2,2-trifluoroethyl)thieno[2,3-d]pyrimidine)

To a mixture of 4-(2,8-diazaspiro[4.5]decan-8-yl)-6-(2,2,2-trifluoroethyl)thieno[2,3-d]pyrimidine (30 mg, 0.084 mmol) in anhydrous CH₃OH (5 mL) was added benzaldehyde (36 mg, 0.336 mmol). The mixture was stirred at 15-22° C. for 5 min. Then NaBH₃CN (21 mg, 0.336 mmol) was added to the mixture. The reaction was stirred at 60° C. for 16 h under N₂ atmosphere. The mixture was quenched with 3 N HCl (5 mL) and concentrated under reduced pressure. The residue was purified by basic preparation RP-HPLC method D and the solution was lyophilized to afford the title compound as yellow oil (22 mg, 58%); LCMS method C: Rₜ=0.63 min; (M+H)⁺=446.9; ¹H NMR (MeOH-d4): δ 8.31 (s, 1H), 7.49 (s, 1H), 7.29 (m, 5H), 3.88 (m, 6H), 3.63 (s, 2H), 2.68 (t, J=6.8 Hz, 2H), 2.52 (s, 2H), 1.78 (t, J=6.8 Hz, 2H), 1.72 (t, J=5.6 Hz, 4H); ¹⁹F NMR (MeOH-d4): δ −67.668, −73.000˜−77.600.

### Example 24

### 4-(2-isobutyl-2,8-diazaspiro[4.5]decan-8-yl)-6-(2,2,2-trifluoroethyl)thieno[2,3-d]pyrimidine

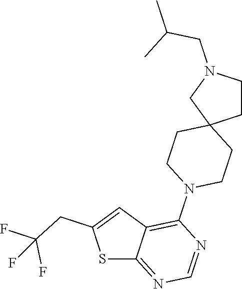 [SMILES: CC(C)CN1CCC2(CCN(c3ncnc4sc(CC(F)(F)F)cc34)CC2)C1]

The title compound was prepared using procedures analogous to those described in Example 23. In step 3, isobutraaldehyde was used instead of benzaldehyde. LCMS method C: Rₜ=0.62 min; (M+H)⁺=412.9; ¹H NMR: (MeOH-d4): δ 8.46 (s, 1H), 7.61 (s, 1H), 4.10-4.15 (m, 2H), 3.90-4.00 (m, 4H), 3.75-3.85 (m, 1H), 3.71 (d, J=12.0 Hz, 1H), 3.05-3.15 (m, 3H), 2.15-2.25 (m, 1H), 2.00-2.15 (m, 2H), 1.80-1.95 (m, 4H), 1.05 (d, J=6.8 Hz, 6H). ¹⁹F NMR: (MeOH-d4): δ −67.661, −77.167.

### Example 25

### 4-(2-isopropyl-2,8-diazaspiro[4.5]decan-8-yl)-6-(2,2,2-trifluoroethyl)thieno[2,3-d]pyrimidine

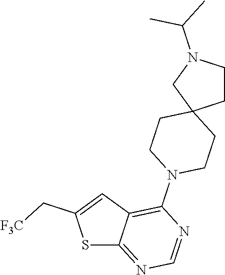 [SMILES: CC(C)N1CCC2(CCN(c3ncnc4sc(CC(F)(F)F)cc34)CC2)C1]

The title compound was prepared using procedures analogous to those described in Example 23. In step 3, acetone was used instead of benzaldehyde. LCMS method C: Rₜ=0.60 min; (M+H)⁺=398.8. ¹H NMR: (MeOH-d4): δ 8.45 (s, 1H), 7.60 (s, 1H), 4.13 (m, 2H), 3.94 (m, 4H), 3.75 (m, 1H), 3.63 (d, J=12 Hz, 1H), 3.47 (m, 1H), 3.35 (s, 1H), 3.09 (d, J=12.0 Hz, 1H), 2.2 (m, 1H), 2.05 (m, 1H), 1.88 (m, 4H), 1.42 (d, J=6.4 Hz, 6H). ¹⁹F NMR: (MeOH-d4): δ −67.696, −77.190.

### Example 26

### 4-methyl-5-((8-(6-(2,2,2-trifluoroethyl)thieno[2,3-d]pyrimidin-4-yl)-2,8-diazaspiro[4.5]decan-2-yl)methyl)-1H-indole-2-carbonitrile

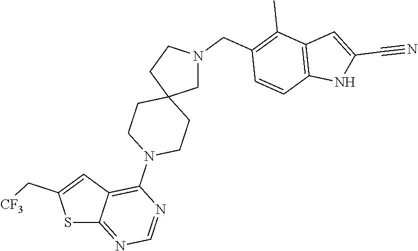 [SMILES: Cc1c(CN2CCC3(CCN(c4ncnc5sc(CC(F)(F)F)cc45)CC3)C2)ccc2[nH]c(C#N)cc12]

The title compound was prepared using procedures analogous to those described in Example 23. In step 3, 5-formyl-4-methyl-1H-indole-2-carbonitrile was used instead of benzaldehyde. The title compound was isolated as a TFA salt. LCMS method B: Rₜ=1.06 min; (M+H)⁺=525.5; ¹H NMR (MeOH-d4): δ (ppm) 8.45 (s, 1H), 7.60 (s, 1H), 7.46 (d, J=8.8 Hz, 1H), 7.39 (d, J=8.8 Hz, 1H), 7.37 (s, 1H), 4.60 (s, 2H), 4.14 (m, 2H), 3.93 (m, 4H), 3.61 (m, 2H), 3.46 (m, 1H), 3.23 (m, 1H), 2.68 (s, 3H), 2.26 (m, 1H), 2.08-1.80 (m, 5H). ¹⁹F NMR (MeOH-d4): δ −67.666 (t, J=10.5 Hz).

### Example 27

### 4-(2-(5,5-dimethyl-4,5-dihydrothiazol-2-yl)-2,8-diazaspiro[4.5]decan-8-yl)-6-(2,2,2-trifluoroethyl)thieno[2,3-d]pyrimidine

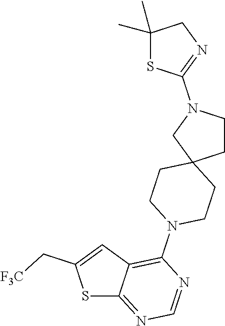 [SMILES: CC1(C)CN=C(N2CCC3(CCN(c4ncnc5sc(CC(F)(F)F)cc45)CC3)C2)S1]

The title compound was synthesized from 4-chloro-6-(2,2,2-trifluoroethyl)thieno[2,3-d]pyrimidine and tert-butyl 2,8-diazaspiro[4.5]decane-2-carboxylate using procedures analogous to those described in Example 10. LCMS method C: Rₜ=0.78 min; (M+H)⁺=470.0; ¹H NMR: (MeOH-d4): δ 8.48 (s, 1H), 7.63 (s, 1H), 3.90-4.20 (m, 6H), 3.85 (s, 2H), 3.65-3.75 (m, 2H), 3.58 (d, J=12.0 Hz, 2H), 2.16 (m, 2H), 1.86 (m, 4H), 1.68 (s, 6H); ¹⁹F NMR: (MeOH-d4): δ −67.638, −77.091.

### Example 28

### 4-(7-((1H-indol-5-yl)methyl)-2,7-diazaspiro[4.4]nonan-2-yl)-6-(2,2,2-trifluoroethyl)thieno[2,3-d]pyrimidine

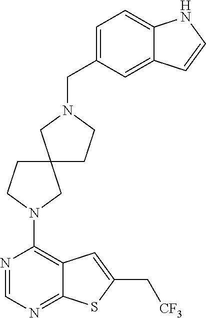 [SMILES: FC(F)(F)Cc1cc2c(N3CCC4(CCN(Cc5ccc6[nH]ccc6c5)C4)C3)ncnc2s1]

### Step 1. tert-butyl 7-(6-(2,2,2-trifluoroethyl)thieno[2,3-d]pyrimidin-4-yl)-2,7-diazaspiro[4.4]nonane-2-carboxylate

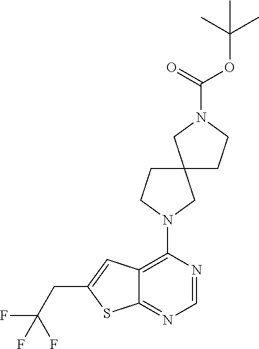 [SMILES: CC(C)(C)OC(=O)N1CCC2(CCN(c3ncnc4sc(CC(F)(F)F)cc34)C2)C1]

A mixture of 4-chloro-6-(2,2,2-trifluoroethyl)thieno[2,3-d]pyrimidine (450 mg, 1.77 mmol), tert-butyl 2,7-diazaspiro[4.4]nonane-2-carboxylate (401 mg, 1.77 mmol) and Na₂CO₃ (564 mg, 5.32 mmol) in CH₃CN (10 mL) was heated at 90° C. for 3 h. The reaction was cooled to 20-24° C. and filtered. The filtrate was concentrated to give tert-butyl 7-(6-(2,2,2-trifluoroethyl)thieno[2,3-d]pyrimidin-4-yl)-2,7-diazaspiro[4.4]nonane-2-carboxylate as a yellow solid (500 mg, 63%), which was used in the next step without purification. LCMS method C: Rₜ=0.73 min; (M+H)⁺=187.1.

### Step 2. 4-(2,7-diazaspiro[4.4]nonan-2-yl)-6-(2,2,2-trifluoroethyl)thieno[2,3-d]pyrimidine

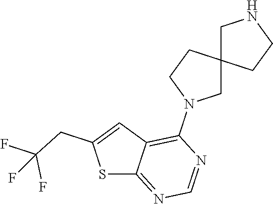 [SMILES: FC(F)(F)Cc1cc2c(N3CCC4(CCNC4)C3)ncnc2s1]

A mixture of tert-butyl 7-(6-(2,2,2-trifluoroethyl)thieno[2,3-d]pyrimidin-4-yl)-2,7-diazaspiro[4.4]nonane-2-carboxylate (250 mg, 0.57 mmol) and HCl/MeOH (3 mL) was stirred at 20-25° C. for 0.5 h. The mixture was concentrated, dissolved in DCM:MeOH (10:1, 50 mL), neutralized by sat. NaHCO₃ (aq. 30 mL). The separated organic layer was dried over anhydrous Na₂SO₄, filtered and concentrated to give 4-(2,7-diazaspiro[4.4]nonan-2-yl)- 6-(2,2,2-trifluoroethyl)thieno[2,3-d]pyrimidine as colorless oil (120 mg, 63% crude yield) which was used in the next step without purification. LCMS method C: Rₜ=0.33 min; (M+H)⁺=343.0.

### Step 3. 4-(7-((1H-indol-5-yl)methyl)-2,7-diazaspiro[4.4]nonan-2-yl)-6-(2,2,2-trifluoroethyl)thieno[2,3-d]pyrimidine

To a solution of 4-(2,7-diazaspiro[4.4]nonan-2-yl)-6-(2,2,2-trifluoroethyl)thieno[2,3-d]pyrimidine (30 mg, 0.088 mmol) in MeOH (3 mL) was added 1H-indole-5-carbaldehyde (14 mg, 0.096 mmol), HOAc (1 drop), and NaBH₃CN (11 mg, 0.175 mmol). The reaction mixture was stirred at 50° C. for 3 h. The mixture was concentrated and purified by acidic preparative RP-HPLC method A to afford the title compound (TFA salt) as a white solid (15 mg, 36%); LCMS method C: Rₜ=0.62 min; (M+H)⁺=471.9; ¹H NMR (methanol-d4 400 MHz): δ 8.43 (d, J=5.6 Hz, 1H), 7.75 (s, 1H), 7.69-7.70 (m, 1H), 7.49 (d, J=8.4 Hz, 1H), 7.33 (d, J=3.2 Hz, 1H), 7.24-7.26 (m, 1H), 6.51 (d, J=2.8 Hz, 1H), 4.51 (s, 2H), 3.91-4.03 (m, 6H), 3.39-3.64 (m, 4H), 2.13-2.29 (m, 4H); ¹⁹F NMR (methanol-d4 400 MHz): δ −67.691, −77.175.

### Example 29

### 4-(7-((1H-indol-6-yl)methyl)-2,7-diazaspiro[4.4]nonan-2-yl)-6-(2,2,2-trifluoroethyl)thieno[2,3-d]pyrimidine

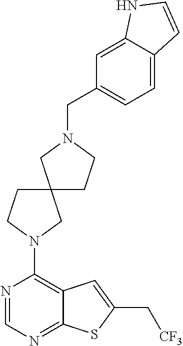 [SMILES: FC(F)(F)Cc1cc2c(N3CCC4(CCN(Cc5ccc6cc[nH]c6c5)C4)C3)ncnc2s1]

The title compound was prepared analogously to the procedures of Example 28, Step 3, from 4-(2,7-diazaspiro[4.4]nonan-2-yl)-6-(2,2,2-trifluoroethyl)thieno[2,3-d]pyrimidine (30 mg, 0.088 mmol) and 1H-indole-6-carbaldehyde (14 mg, 0.096 mmol), and purified by basic preparative RP-HPLC method D to afford the title compound as a white solid (6.1 mg,15%). LCMS method D: Rₜ=1.70 min; (M+H)⁺=472.1; ¹H NMR (MeOH-d4): δ 8.27 (s, 1H), 7.75 (s, 1H), 7.60 (s, 1H), 7.49 (d, J=8.0 Hz, 1H), 7.37 (s, 1H), 7.21(d, J=3.2 Hz, 1H), 7.03 (d, J=1.2 Hz, 1H), 7.01 (d, J=1.2 Hz, 1H), 6.41 (d, J=3.2Hz, 1H),4.60 (m, 1H), 3.76-3.87 (m, 7H), 2.64-2.87 (m, 4H), 2.04-2.08 (m, 2H), 1.90-1.94 (m, 2H). ¹⁹F NMR (MeOH-d4): δ −67.739.

### Example 30

### 4-methyl-5-((7-(6-(2,2,2-trifluoroethyl)thieno[2,3-d]pyrimidin-4-yl)-2,7-diazaspiro[4.4]nonan-2-yl)methyl)-1H-indole-2-carbonitrile (R and S Enantiomers)

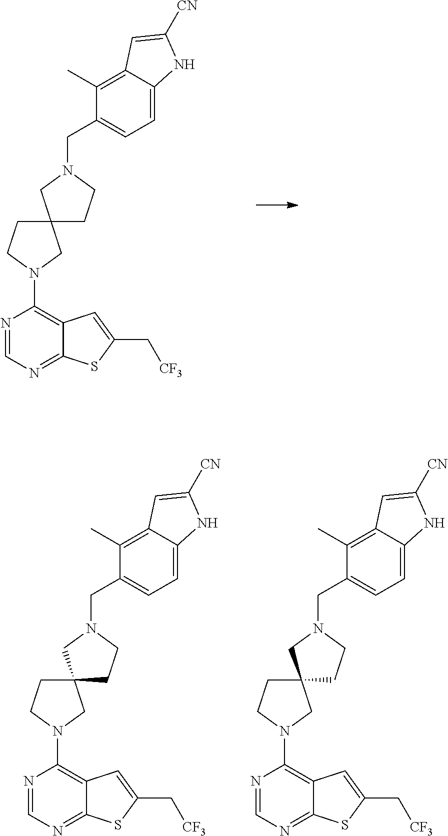 [SMILES: Cc1c(CN2CCC3(CCN(c4ncnc5sc(CC(F)(F)F)cc45)C3)C2)ccc2[nH]c(C#N)cc12 ; Cc1c(CN2CC[C@@]3(CCN(c4ncnc5sc(CC(F)(F)F)cc45)C3)C2)ccc2[nH]c(C#N)cc12 ; Cc1c(CN2CC[C@]3(CCN(c4ncnc5sc(CC(F)(F)F)cc45)C3)C2)ccc2[nH]c(C#N)cc12]

The title compounds were synthesized according to the procedures of Example 28. In step 3, 5-formyl-4-methyl-1H-indole-2-carbonitrile was utilized instead of benzaldehyde. The racemic mixture was purified by SFC (Column: Chiral IC-3 150×4.6 mm I.D., 3 μm Mobile phase: 40% of isopropanol (0.05% DEA) in CO₂, Flow rate: 2.5 mL/min Column temperature: 40° C.) to afford each enantiomer as a white solid.

Isomer 1 (Example 30a): LCMS method C: Rₜ=0.65 min; (M+H)⁺=511.1; ¹H NMR (MeOH-d4,): δ 8.28 (s, 1H), 7.62 (s, 1H), 7.33 (d, J=8.8 Hz, 1H), 7.28 (s, 1H), 7.23 (d, J=8.8 Hz, 1H), 3.77-3.92 (m, 8H), 2.62-2.75 (m, 4H), 2.60 (s, 3H), 2.09 (s, 2H), 1.91-1.95 (m, 2H); ¹⁹F NMR (MeOH-d4,): δ −67.752; SFC EE>99.9%, t_{R}=5.54 min.

Isomer 2 (Example 30b): LCMS method C: Rₜ=0.65 min; (M+H)⁺=511.1; ¹H NMR (MeOH-d4,) δ 8.28 (s, 1H), 7.62 (s, 1H), 7.33 (d, J=8.8 Hz, 1H), 7.28 (s, 1H), 7.23 (d, J=8.8 Hz, 1H), 3.77-3.92 (m, 8H), 2.62-2.75 (m, 4H), 2.60 (s, 3H), 2.09 (s, 2H), 1.91-1.95 (m, 2H); ¹⁹F NMR (MeOH-d4): δ −67.752; SFC EE=99.16%, t_{R}=6.70 min.

### Example 31

### 4-(7-((Tetrahydrofuran-3-yl)methyl)-2,7-diazaspiro[4.4]nonan-2-yl)-6-(2,2,2-trifluoroethyl)thieno[2,3-d]pyrimidine

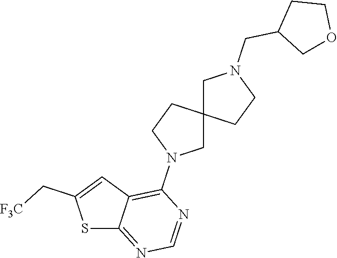 [SMILES: FC(F)(F)Cc1cc2c(N3CCC4(CCN(CC5CCOC5)C4)C3)ncnc2s1]

The title compound was prepared as a TFA salt using procedures analogous to those described in Example 28. In step 3, 3-tetrahydrofuran carbaldehyde (CAS registry number 79710-86-4) was used. LCMS method B: Rₜ=0.80 min; (M+H)⁺=427.5; ¹H NMR (MeOH-d4): δ 8.44 (s, 1H), 7.71 (s, 1H), 3.62-4.10 (m, 12H), 3.51 (m, 1H), 3.34 (m, 3H), 2.64 (m, 1H), 2.24 (m, 5H), 1.72 (m, 1H). ¹⁹F NMR (MeOH-d4): δ −67.722.

### Example 32

### 4-(7-((4,4-difluorocyclohexyl)methyl)-2,7-diazaspiro[4.4]nonan-2-yl)-6-(2,2,2-trifluoroethyl)thieno[2,3-d]pyrimidine

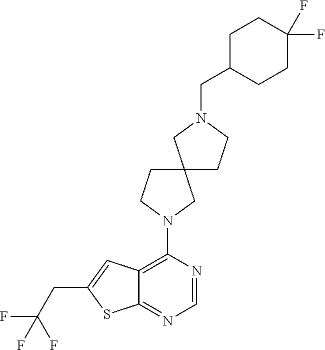 [SMILES: FC(F)(F)Cc1cc2c(N3CCC4(CCN(CC5CCC(F)(F)CC5)C4)C3)ncnc2s1]

The title compound was prepared as a TFA salt using procedures analogous to those described in Example 28. In step 3, 4,4-difluorocyclohexane carbaldehyde (CAS registry number 265108-36-9) was used. LCMS method B: Rₜ=0.95 min; (M+H)⁺=475.6.

### Example 33

### 4-((7-(6-(2,2,2-trifluoroethyl)thieno[2,3-d]pyrimidin-4-yl)-2,7-diazaspiro[4.4]nonan-2-yl)methyl)benzonitrile

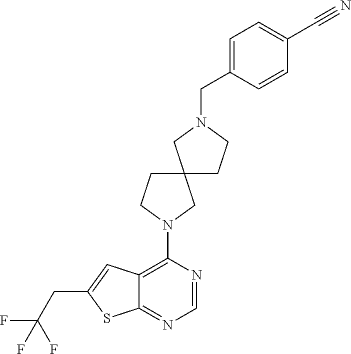 [SMILES: N#Cc1ccc(CN2CCC3(CCN(c4ncnc5sc(CC(F)(F)F)cc45)C3)C2)cc1]

The title compound was prepared using procedures analogous to those described in Example 28. In step 3, 4-cyanobenzaldehyde was used. The title compound was isolated as a TFA salt. LCMS method B: Rₜ=0.98 min; (M+H)⁺=458.5; ¹H NMR (MeOH-d4): δ 8.42 (s, 1H), 7.85 (d, J=8.4 Hz, 2H), 7.73 (d, J=8.4 Hz, 2H), 7.68 (s, 1H), 4.55 (s, 2H), 4.02 (m, 4H), 3.92 (q, J=10.4 Hz, 2H), 3.60 (m, 4H), 2.26 (m, 4H). ¹⁹F NMR (MeOH-d4): δ −67.715 (t, J=10.4 Hz).

### Example 34

### N-(4-((7-(6-(2,2,2-trifluoroethyl)thieno[2,3-d]pyrimidin-4-yl)-2,7-diazaspiro[4.4]nonan-2-yl)methyl)phenyl)acetamide

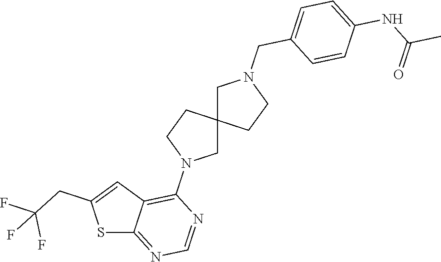 [SMILES: CC(=O)Nc1ccc(CN2CCC3(CCN(c4ncnc5sc(CC(F)(F)F)cc45)C3)C2)cc1]

The title compound was prepared using procedures analogous to those described in Example 28. In step 3, 4-acetamido benzaldehyde was used. The title compound was isolated as a TFA salt. LCMS method B: Rₜ=0.94 min; (M+H)⁺=490.6; ¹H NMR (MeOH-d4): δ (ppm) 8.61 (s, 1H), 8.03 (br, 1H), 7.88 (s, 1H), 7.67 (m, 2H), 7.57 (m, 2H), 4.44 (m, 2H), 4.28 (m, 2H), 3.92-4.14 (m, 4H), 3.34-3.78 (m, 4H), 2.16-2.46 (m, 4H), 2.13 (s, 3H). ¹⁹F NMR (MeOH-d4): δ −67.648.

### Example 35

### 3-((7-(6-(2,2,2-Trifluoroethyl)thieno[2,3-d]pyrimidin-4-yl)-2,7-diazaspiro[4.4]nonan-2-yl)methyl)benzonitrile

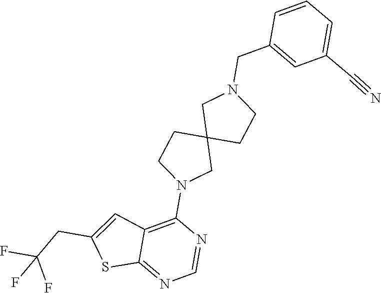 [SMILES: N#Cc1cccc(CN2CCC3(CCN(c4ncnc5sc(CC(F)(F)F)cc45)C3)C2)c1]

The title compound was prepared using procedures analogous to those described in Example 28. In step 3, 3-cyanobenzaldehyde was utilized. The title compound was isolated as a TFA salt. LCMS method B: Rₜ=0.98 min; (M+H)⁺=458.5.

### Example 36

### 4-(7-(2-chloro-4-(methylsulfonyl)benzyl)-2,7-diazaspiro[4.4]nonan-2-yl)-6-(2,2,2-trifluoroethyl)thieno[2,3-d]pyrimidine

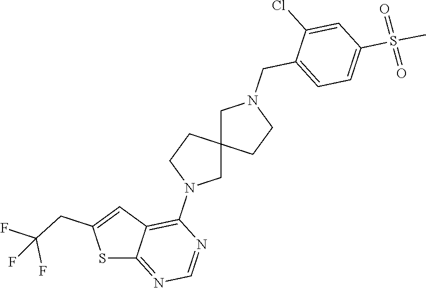 [SMILES: CS(=O)(=O)c1ccc(CN2CCC3(CCN(c4ncnc5sc(CC(F)(F)F)cc45)C3)C2)c(Cl)c1]

The title compound was prepared using procedures analogous to those described in Example 28. In step 3, 2-chloro-4-methylsulfonyl benzaldehyde (CAS registry number 101349-95-5) was used. The title compound was isolated as a TFA salt. LCMS method B: Rₜ=1.03 min; (M+H)⁺=545.5.

### Example 37

### 6-(2,2,2-Trifluoroethyl)-4-(7-(((1r,4r)-4-(trifluoromethyl)cyclohexyl)methyl)-2,7-diazaspiro[4.4]nonan-2-yl)thieno[2,3-d]pyrimidine

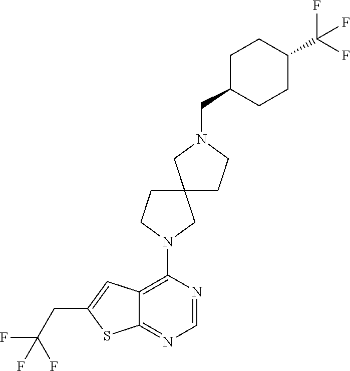 [SMILES: FC(F)(F)Cc1cc2c(N3CCC4(CCN(C[C@H]5CC[C@H](C(F)(F)F)CC5)C4)C3)ncnc2s1]

The title compound was prepared using procedures analogous to those described in Example 28. In step 3, trans-4-trifluoromethyl cyclohexylcarbaldehyde (CAS registry number registry number 133261-34-4; Journal of the American Chemical Society, 123(23), 5414-5417; 2001) was used. The title compound was isolated as a TFA salt. LCMS method B: Rₜ=1.18 min; (M+H)⁺=507.6.

### Example 38

### N-(4-((7-(6-(2,2,2-trifluoroethyl)thieno[2,3-d]pyrimidin-4-yl)-2,7-diazaspiro[4.4]nonan-2-yl)methyl)phenyl)methanesulfonamide

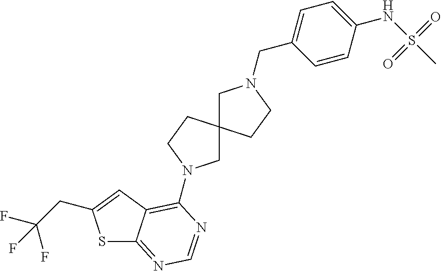 [SMILES: CS(=O)(=O)Nc1ccc(CN2CCC3(CCN(c4ncnc5sc(CC(F)(F)F)cc45)C3)C2)cc1]

The title compound was prepared using procedures analogous to those described in Example 28. In step 3, N-(4-formylphenyl)methanesulfonamide was utilized. The title compound was isolated as a TFA salt. LCMS method B: Rₜ=0.96 min; (M+H)⁺=526.6; ¹H NMR (MeOH-d4): δ 8.40 (s, 1H), 7.66 (s, 1H), 7.51 (d, J=8.4 Hz, 2H), 7.34 (d, J=8.4 Hz, 2H), 4.41 (s, 2H), 4.02 (m, 2H), 3.92 (m, 2H), 3.91 (q, J=10.4 Hz, 2H), 3.66 (m, 2H), 3.45 (m, 2H), 3.00 (s, 3H), 2.38-2.04 (m, 4H). ¹⁹F NMR (MeOH-d4): δ −67.722 (t, J=10.4 Hz).

### Example 39

### 5-((7-(6-(2,2,2-trifluoroethyl)thieno[2,3-d]pyrimidin-4-yl)-2,7-diazaspiro[4.4]nonan-2-yl)methyl)-1H-benzo[d]imidazol-2(3H)-one

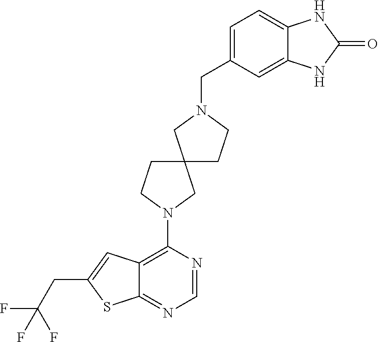 [SMILES: O=c1[nH]c2ccc(CN3CCC4(CCN(c5ncnc6sc(CC(F)(F)F)cc56)C4)C3)cc2[nH]1]

The title compound was prepared using procedures analogous to those described in Example 28. In step 3, 2-oxo-2,3-dihydro-1H-benzo[d]imidazole-5-carbaldehyde (CAS registry number 106429-59-8) was used. LCMS method C: Rₜ=0.61 min; (M+H)⁺=489.1; ¹H NMR (MeOD-d4): δ 8.44 (s, 1H), 7.70 (S, 1H), 7.20-7.28 (m, 2H), 7.11-7.17 (m, 1H), 4.47 (s, 2H), 3.87-4.13 (m, 6H), 3.38-3.75 (m, 4H), 2.05-2.42 (m, 4H); ¹⁹FNMR (MeOD-d4): δ −67.69, −77.18.

### Example 40

### 6-((7-(6-(2,2,2-trifluoroethyl)thieno[2,3-d]pyrimidin-4-yl)-2,7-diazaspiro[4.4]nonan-2-yl)methyl)benzo[d]oxazol-2(3H)-one

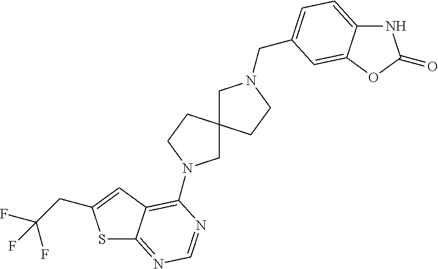 [SMILES: O=c1[nH]c2ccc(CN3CCC4(CCN(c5ncnc6sc(CC(F)(F)F)cc56)C4)C3)cc2o1]

The title compound was prepared using procedures analogous to those described in Example 28. In step 3, 2-oxo-2,3-dihydrobenzo[d]oxazole-6-carbaldehyde (CAS registry number 54903-15-0) was used. LCMS method B: Rₜ=1.04 min; (M+H)⁺=490.5.

### Example 41

### 1-methyl-5-((7-(6-(2,2,2-trifluoroethyl)thieno[2,3-d]pyrimidin-4-yl)-2,7-diazaspiro[4.4]nonan-2-yl)methyl)-1H-benzoldlimidazol-2(3H)-one

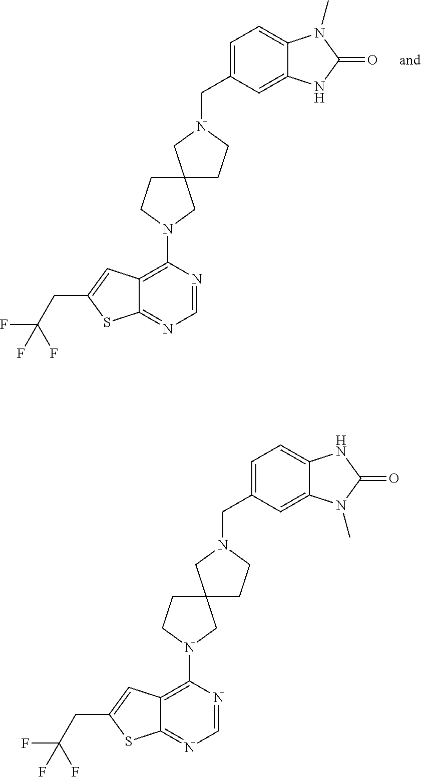 [SMILES: Cn1c(=O)[nH]c2cc(CN3CCC4(CCN(c5ncnc6sc(CC(F)(F)F)cc56)C4)C3)ccc21 ; Cn1c(=O)[nH]c2ccc(CN3CCC4(CCN(c5ncnc6sc(CC(F)(F)F)cc56)C4)C3)cc21]

The title compounds were prepared as a mixture using procedures analogous to those described in Example 28, Step 3 starting from a mixture of 1-methyl-2-oxo-2,3-dihydro-1H-benzo[d]imidazole-5-carbaldehyde and 3-methyl-2-oxo-2,3-dihydro-1H-benzo[d]imidazole-5-carbaldehyde. LCMS method C: Rₜ=0.63 min; (M+H)⁺=503.1; ¹H NMR (MeOD-d4): δ 8.28 (s, 1H), 7.61 (s, 1H), 7.00-7.16 (m, 3H), 3.68-3.94 (m, 8H), 3.39 (s, 3H), 2.57-2.85 (m, 4H), 2.04-2.15 (m, 2H), 1.92-1.96 (m, 2H); ¹⁹F NMR (MeOD-d4): δ −67.71.

### Example 42

### N-(3-methyl-4-((7-(6-(2,2,2-trifluoroethyl)thieno[2,3-d]pyrimidin-4-yl)-2,7-diazaspiro[4.4]nonan-2-yl)methyl)phenyl)acetamide

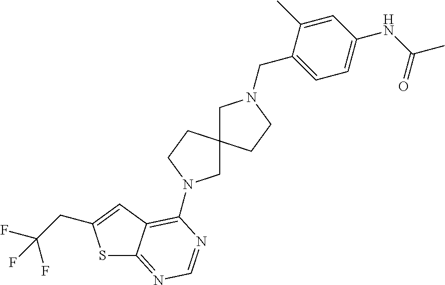 [SMILES: CC(=O)Nc1ccc(CN2CCC3(CCN(c4ncnc5sc(CC(F)(F)F)cc45)C3)C2)c(C)c1]

The title compound was prepared using procedures analogous to those described in Example 28. In step 3, 4-acetamido-2-methyl benzaldehyde (CAS registry number 84257-50-1) was used. LCMS method D: Rₜ=1.99 min; (M+H)⁺=464.1. ¹H NMR (MeOD-d₄): δ 8.27 (s, 1H), 7.61 (s, 1H), 7.32 (s, 2H), 7.20 (d, J=8.8 Hz, 1H), 3.75-3.91 (m, 6H), 3.62 (s, 2H), 2.74-2.77 (s, 1H), 2.66-2.68(s, 2H), 2.54-2.57 (m, 1H), 2.36 (s, 3H), 2.01-2.12 (m, 5H), 1.88-1.93 (m, 2H). ¹⁹F NMR (MeOD-d4): δ −67.74.

### Example 43

### 4-(7-benzyl-2,7-diazaspiro[4.4]nonan-2-yl)-6-(2,2,2-trifluoroethyl)thieno[2,3-d]pyrimidine

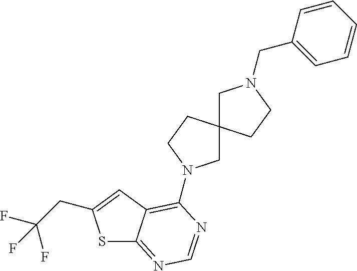 [SMILES: FC(F)(F)Cc1cc2c(N3CCC4(CCN(Cc5ccccc5)C4)C3)ncnc2s1]

The title compound was prepared using procedures analogous to those described in Example 28 using benzaldehyde in Step 3. The title compound was isolated as a TFA salt. LCMS method B: Rₜ=0.95 min; (M+H)⁺=433.5; ¹H NMR (MeOH-d4): δ 8.46 (s, 1H), 7.71 (s, 1H), 7.44-7.60 (m, 5H), 4.46 (s, 2H), 3.86-4.18 (m, 6H), 3.36-3.76 (m, 4H), 2.27 (m, 4H). ¹⁹F NMR (MeOH-d4): δ −67.690 (t, J=10.4 Hz).

### Example 44

### N-(3-chloro-4-((7-(6-(2,2,2-trifluoroethyl)thieno[2,3-d]pyrimidin-4-yl)-2,7-diazaspiro[4.4]nonan-2-yl)methyl)phenyl)acetamide

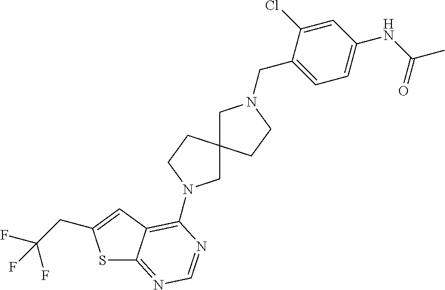 [SMILES: CC(=O)Nc1ccc(CN2CCC3(CCN(c4ncnc5sc(CC(F)(F)F)cc45)C3)C2)c(Cl)c1]

The title compound was prepared using procedures analogous to those described in Example 28. In step 3, 4-acetamido-3-chloro benzaldehyde (CAS registry number 69828-55-3; ChemMedChem, 4(3), 339-351; 2009) was used. LCMS method C: Rₜ=0.65 min; (M+H)⁺=524.1; ¹H NMR (MeOH-d4): δ 8.38 (s, 1H), 7.95 (d, J=1.6 Hz, 1H), 7.52-7.71 (m, 3H), 4.60 (s, 2H), 3.84-4.13 (m, 6H), 3.43-3.81 (m, 4H), 2.28 (m, 4H), 2.14 (s, 3H); ¹⁹F NMR (MeOH-d4): δ −67.694, −77.126.

### Example 45

### N-(3-fluoro-4-((7-(6-(2,2,2-trifluoroethyl)thieno[2,3-d]pyrimidin-4-yl)-2,7-diazaspiro[4.4]nonan-2-yl)methyl)phenyl)acetamide

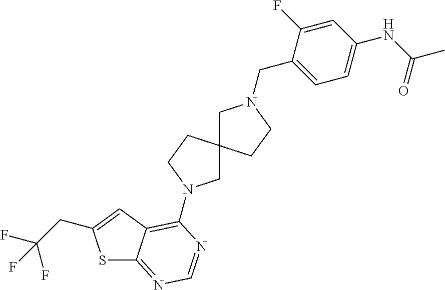 [SMILES: CC(=O)Nc1ccc(CN2CCC3(CCN(c4ncnc5sc(CC(F)(F)F)cc45)C3)C2)c(F)c1]

### Step 1: 4-amino-2-fluorobenzaldehyde

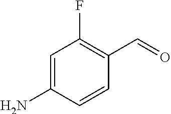

To a solution of 2-fluoro-4-nitrobenzaldehyde (CAS registry number 157701-72-9) (50 mg, 0.3 mmol) in EtOH (3 mL) and H₂O (3 mL) were added Fe power (101 mg, 1.8 mmol) and NH₄Cl (95 mg, 1.8 mmol). Then the reaction was heated at 60° C. for 1 h under N₂. TLC confirmed that starting material was consumed and a new product was formed. The mixture was diluted with H₂O (30 mL) and extracted with EtOAc (30 mL×2). The combined organic layer was dried over Na₂SO₄, filtered and concentrated to give 4-amino-2-fluorobenzaldehyde as a yellow solid (35 mg, 85%), which was used for the next step without purification. ¹H NMR (CDCl₃ 400 MHz): δ 10.08 (s, 1H), 7.68 (t, J=8.0 Hz, 1H), 6.45 (dd, J=8.4, 2.0 Hz, 1H), 6.31 (dd, J=12.4, 2.4 Hz, 1H), 4.73 (s, 1H), 4.40 (s, 1H).

### Step 2: N-(3-fluoro-4-formylphenyl)acetamide

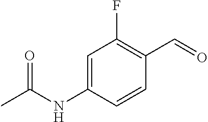

To a solution of 4-amino-2-fluorobenzaldehyde (35 mg, 0.22 mmol) in DCM (3 mL), was added Ac₂O (22 mg, 0.22 mmol) and TEA (43 mg, 0.44 mmol). The reaction was stirred at 24-30° C. for 16 h. TLC (petroleum ether:ethyl acetate=5:1) confirmed that the starting material was consumed and a new product was formed. The mixture was diluted with H₂O (5 mL) and extracted with EtOAc (10 mL×2). The combined organic layer was dried over Na₂SO₄, filtered and concentrated to give the crude, which was purified by preparative TLC (petroleum ether:ethyl acetate=3:1) to afford N-(3-fluoro-4-formylphenyl)acetamide as a yellow solid (25 mg, 56%); (MH)⁺=182.0.

### Step 3: (N-(3-fluoro-4-((7-(6-(2,2,2-trifluoroethypthieno[2,3-d]pyrimidin-4-yl)-2,7-diazaspiro[4.4]nonan-2-yl) methyl)phenyl)acetamide)

To a solution of 3-((7-(6-(2,2,2-trifluoroethyl)thieno[2,3-d]pyrimidin-4-yl)-2,7-diazaspiro[4.4]nopnan-2-yl)-methyl)aniline (30 mg, 0.09 mmol) in MeOH (5 mL) were added N-(3-fluoro-4-formylphenyl)acetamide (16 mg, 0.09 mmol) and HOAc (2 drops). Then NaBH₃CN (12 mg, 0.18 mmol) was added. The reaction was stirred at 50° C. for 1 h. LCMS showed about 82% of title compound was detected. The mixture was concentrated and purified by preparative RP-HPLC Method A to afford the title compound (N-(3-fluoro-4-((7-(6-(2,2,2-trifluoroethyl)thieno[2,3-d]pyrimidin-4-yl)-2,7-diazaspiro[4.4]nonan-2-yl)methyl)phenyl)acetamide, TFA salt) as a yellow solid (27.0 mg, 61%). LCMS method C: Rₜ=0.64 min; (M+H)⁺=508.1; ¹H NMR (MeOH-d4): δ8.24 (s, 1H), 7.68-7.77 (s, 2H), 7.49-7.53 (m, 1H), 7.34-7.36 (m, 1H), 4.49 (d, 2H), 3.88-4.03 (m, 6H), 3.52-3.69 (s, 4H), 2.27 (s, 4H), 2.14 (s, 3H); ¹⁹F NMR (MeOH-d4): δ−115.309, −77.179, −67.701.

### Example 46

### Methyl ((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)carbamate

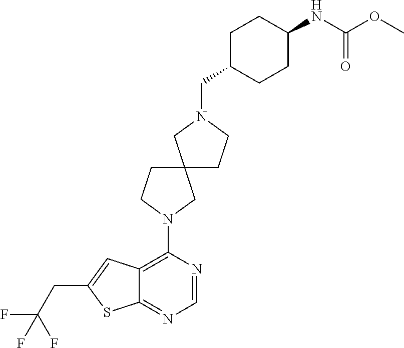 [SMILES: COC(=O)N[C@H]1CC[C@H](CN2CCC3(CCN(c4ncnc5sc(CC(F)(F)F)cc45)C3)C2)CC1]

### Step 1. tert-butyl ((1r,4r)-4-formylcyclohexyl)carbamate

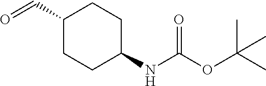 [SMILES: CC(C)(C)OC(=O)N[C@H]1CC[C@H](C=O)CC1]

To a solution of tert-butyl ((1r,4r)-4-(hydroxymethyl)cyclohexyl)carbamate in 1,2-dichloroethane (DCE) (2 mL), there was added Dess-Martin Periodinane (264 mg, 0.63 mmol). The resulting mixture was heated at 80° C. for 10 min in a CEM microwave. After cooling down, 1N NaOH (5 mL) was added to the solution, and extracted with DCM (5 mL×2), the combined organic layers were dried over Na₂SO₄, filtered and concentrated carefully under vacuum, the resulted colourless liquid was used for the next steps without purification. LCMS method B: Rₜ=1.48 min.

### Step 2. tert-butyl ((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)carbamate

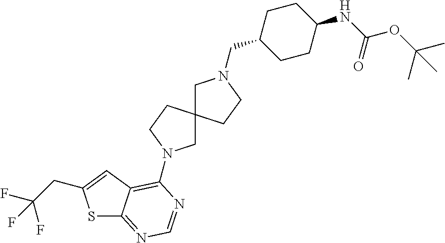 [SMILES: CC(C)(C)OC(=O)N[C@H]1CC[C@H](CN2CCC3(CCN(c4ncnc5sc(CC(F)(F)F)cc45)C3)C2)CC1]

To a solution of 4-(2,7-diazaspiro[4.4]nonan-2-yl)-6-(2,2,2-trifluoroethyl)thieno[2,3-d]pyrimidine hydrochloride (75 mg, 0.20 mmol) (as prepared in Example 28, Step 2, without neutralization) in MeOH (1.5 mL) there was added NaOAc (16 mg, 0.20 mmol), followed by tert-butyl ((1r,4r)-4-formylcyclohexyl)carbamate (0.30 mmol). The resulting solution was stirred at RT for 15 minutes before NaCNBH₃ (19 mg, 0.3 mmol) was added to the solution. The reaction was done within one hour by LC-MS. Methanol was removed by vacuum, saturated NaHCO₃ solution (10 mL) was added to the residue, and the mixture extracted by EtOAc (10 mL×3). The combined organic layers were washed with brine (10 mL), dried over Na₂SO₄, filtered and concentrated under vacuum. The residue was purified by ISCO column (silica) eluting with 0 to 10% MeOH in DCM to give tert-butyl ((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)carbamate (67 mg, 61% in two steps). LCMS method B: Rₜ=1.23 min. [M+H]⁺=554.5.

### Step 3. (1r,4r)-4-((7-(6-(2,2,2-trifluoroethypthieno[2,3-d]pyrimidin-4-yl)-2,7-diazaspiro[4.4]nonan-2-yl)methyl)cyclohexan-1-amine

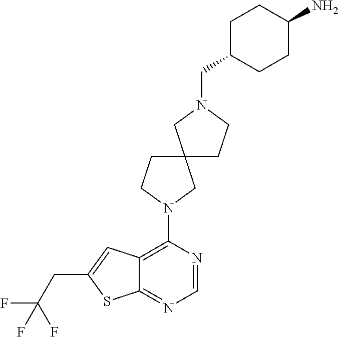 [SMILES: N[C@H]1CC[C@H](CN2CCC3(CCN(c4ncnc5sc(CC(F)(F)F)cc45)C3)C2)CC1]

To a solution of tert-butyl ((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)carbamate (67 mg, 0.12 mmol) in DCM (1.5 mL), there was added trifluoroacetic acid (0.5 mL). The resulting solution was stirred at RT for 1 h, and LC-MS indicated the reaction went to completion. Solvent was removed to give (1r,4r)-4-((7-(6-(2,2,2-trifluoroethyl)thieno[2,3-d]pyrimidin-4-yl)-2,7-diazaspiro[4.4]nonan-2-yl)methyl)cyclohexan-1-amine as a gummy oil, which was used for the next step without purification. LCMS method B: Rₜ=1.04 min; [M+H]⁺=454.5.

### Step 4. methyl ((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)carbamate

To a solution of (1r,4r)-4-((7-(6-(2,2,2-trifluoroethyl)thieno[2,3-d]pyrimidin-4-yl)-2,7-diazaspiro[4.4]nonan-2-yl)methyl)cyclohexan-1-amine (0.05 mmol) in DCM (1 mL), there was added Et₃N (100 μL, excess). The resulting solution was cooled to 0° C. and methyl carbonochloridate (0.06 mmol) was added to the solution carefully. The reaction was warmed to RT, and stirred overnight. Solvent was removed, and residue was purified through Gilson RP-HPLC Method B to give the title compound as a TFA salt; LC-MS method B: Rₜ=1.09 min; (M+H)⁺=512.6.

### Example 47

### N-((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)acetamide

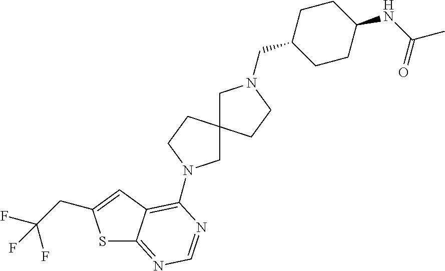 [SMILES: CC(=O)N[C@H]1CC[C@H](CN2CCC3(CCN(c4ncnc5sc(CC(F)(F)F)cc45)C3)C2)CC1]

The title compound was prepared using procedures analogous to those described in Example 46. In step 4, acetyl chloride was used. The title compound was isolated as a TFA salt. LCMS method B: Rₜ=0.98 min; (M+H)⁺=496.5.

### Example 48

### N-((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)methanesulfonamide

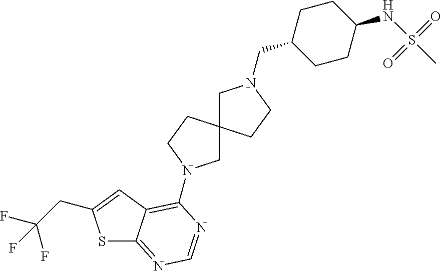 [SMILES: CS(=O)(=O)N[C@H]1CC[C@H](CN2CCC3(CCN(c4ncnc5sc(CC(F)(F)F)cc45)C3)C2)CC1]

The title compound was prepared using procedures analogous to those described in Example 46. In step 4, methanesulfonyl chloride was used. The title compound was isolated as a TFA salt. LCMS method B: Rₜ=1.04 min; (M+H)⁺=532.7; ¹H NMR (MeOH-d4): δ 8.38 (s, 1H), 7.66 (s, 1H), 3.76-4.08 (m, 7H), 3.14-3.30 (m, 4H), 2.94 (s, 3H), 2.28 (m, 4H), 2.08 (m, 2H), 1.92 (m, 2H), 1.76 (m, 1H), 1.35 (m, 2H), 1.19 (m, 2H). ¹⁹F NMR (MeOH-d4): δ −67.722 (t, J=10.4 Hz).

### Example 49

### N-((1s,4s)-4-((7-(6-(2,2,2-trifluoroethyl)thieno[2,3-d]pyrimidin-4-yl)-2,7-diazaspiro[4.4]nonan-2-yl)methyl)cyclohexyl)methanesulfonamide

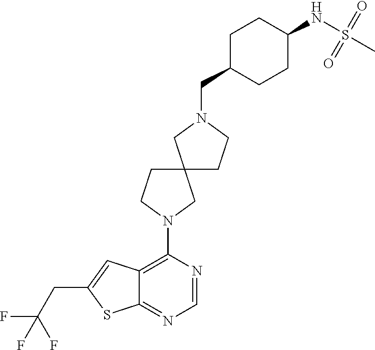 [SMILES: CS(=O)(=O)N[C@H]1CC[C@@H](CN2CCC3(CCN(c4ncnc5sc(CC(F)(F)F)cc45)C3)C2)CC1]

The title compound was synthesized from 4-(2,7-diazaspiro[4.4]nonan-2-yl)-6-(2,2,2-trifluoroethyl)thieno[2,3-d]pyrimidine (see Example 28, Step 2) and tert-butyl ((1s,4s)-4-(hydroxymethyl)cyclohexyl)carbamate (CAS registry number 223131-01-9) using procedures analogous to those described in Example 46, Steps 1-4. In Step 4, methanesulfonic anhydride was used instead of methyl carbonochloridate. The title compound was isolated as a TFA salt. LCMS method B: Rₜ=1.04 min; (M+H)⁺=532.6.

### Example 50

### N-((1s,4s)-4-((7-(6-(2,2,2-Trifluoroethyl)thieno[2,3-d]pyrimidin-4-yl)-2,7-diazaspiro[4.4]nonan-2-yl)methyl)cyclohexyl)acetamide

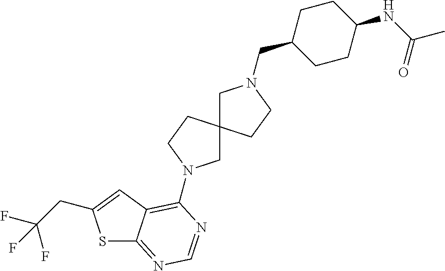 [SMILES: CC(=O)N[C@H]1CC[C@@H](CN2CCC3(CCN(c4ncnc5sc(CC(F)(F)F)cc45)C3)C2)CC1]

The title compound was synthesized from 4-(2,7-diazaspiro[4.4]nonan-2-yl)-6-(2,2,2-trifluoroethyl)thieno[2,3-d]pyrimidine hydrochloride and tert-butyl ((1s,4s)-4-(hydroxymethyl)cyclohexyl)carbamate using procedures analogous to those described in Example 46, Steps 1 through 4. In Step 4, acetyl chloride was used instead of methyl carbonochloridate. The title compound was isolated as a TFA salt. LCMS method B: Rₜ=0.99 min; (M+H)⁺=496.6.

### Example 51

### Methyl ((1s,4s)-4-((7-(6-(2,2,2-trifluoroethyl)thieno[2,3-d]pyrimidin-4-yl)-2,7-diazaspiro[4.4]nonan-2-yl)methyl)cyclohexyl)carbamate

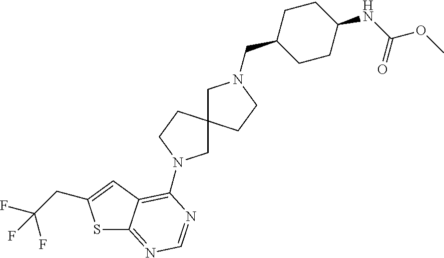 [SMILES: COC(=O)N[C@H]1CC[C@@H](CN2CCC3(CCN(c4ncnc5sc(CC(F)(F)F)cc45)C3)C2)CC1]

The title compound was synthesized from 4-(2,7-diazaspiro[4.4]nonan-2-yl)-6-(2,2,2-trifluoroethyl)thieno[2,3-d]pyrimidine hydrochloride and tert-butyl ((1s,4s)-4-(hydroxymethyl)cyclohexyl)carbamate using procedures analogous to those described in Example 46, Steps 1 through 4. The title compound was isolated as a TFA salt. LCMS method B: Rₜ=1.08 min; (M+H)⁺=512.6.

### Example 52

### N-(3-((7-(6-(2,2,2-trifluoroethyl)thieno[2,3-d]pyrimidin-4-yl)-2,7-diazaspiro[4.4]nonan-2-yl)methyl)phenyl)acetamide

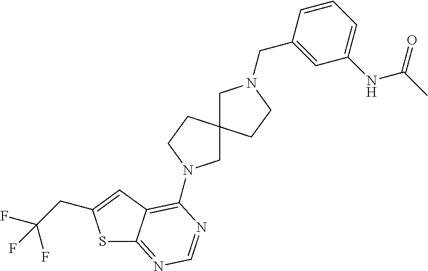 [SMILES: CC(=O)Nc1cccc(CN2CCC3(CCN(c4ncnc5sc(CC(F)(F)F)cc45)C3)C2)c1]

### Step 1. 4-(7-(3-nitrobenzyl)-2,7-diazaspiro[4.4]nonan-2-yl)-6-(2,2,2-trifluoroethyl)thieno[2,3-d]pyrimidine

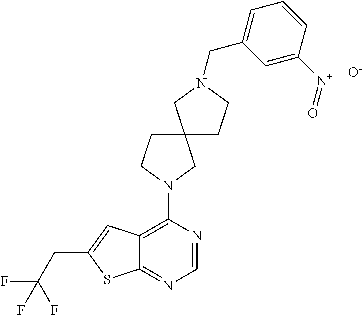

To a solution of 4-(2,7-diazaspiro[4.4]nonan-2-yl)-6-(2,2,2-trifluoroethyl)thieno[2,3-d]pyrimidine (see Example 28, Step 2) (100 mg, 0.29 mmol) in MeOH (10 mL) were added 3-nitrobenzaldehyde (44 mg, 0.29 mmol), NaCNBH₃ (1 eq) and HOAc (1 drop). Then the reaction was heated at 50° C. for 1 h. The mixture was diluted with H₂O (50 mL) and extracted with EtOAc (30 mL×2). The combined organic layers were dried over Na₂SO₄, filtered and concentrated to give the crude product, which was purified by chromatography column (silica gel, eluting with PE:EtOAc=2:1 to DCM:MeOH=10:1) to afford 4-(7-(3-nitrobenzyl)-2,7-diazaspiro[4.4]nonan-2-yl)-6-(2,2,2-trifluoroethyl)thieno[2,3-d]pyrimidine as a colorless oil (120 mg); LCMS method C: Rₜ=0.67 min; (M+H)⁺=478.1.

### Step 2. 3-((7-(6-(2,2,2-trifluoroethyl)thieno[2,3-d]pyrimidin-4-yl)-2,7-diazaspiro[4.4]nonan-2-yl)methyl)andine

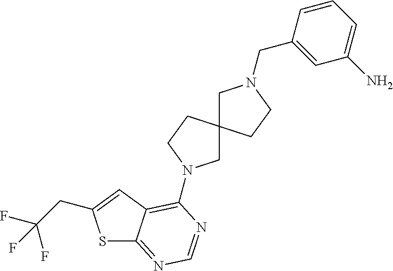 [SMILES: Nc1cccc(CN2CCC3(CCN(c4ncnc5sc(CC(F)(F)F)cc45)C3)C2)c1]

A mixture of 4-(7-(3-nitrobenzyl)-2,7-diazaspiro[4.4]nonan-2-yl)-6-(2,2,2-trifluoroethyl)thieno[2,3-d]-pyrimidine (50 mg, 0.1 mmol) and Pd/C (5 mg, 10%) in EtOAc (5 mL) was stirred under 15 psi of H₂ at 24-29° C. for 3 h. TLC (PE:EtOAc=1:1) confirmed that starting material was consumed and a new spot was formed. The mixture was filtered through Celite, and the filtrate was concentrated to afford 3-((7-(6-(2,2,2-trifluoroethyl)thieno[2,3-d]pyrimidin-4-yl)-2,7-diazaspiro[4.4]nonan-2-yl)methyl)aniline as a colorless oil (40 mg), which was used in the next step without purification. LCMS method C: Rₜ=1.90 min; (M+H)⁺=448.1.

### Step 3. N-(3-((7-(6-(2,2,2-trifluoroethyl)thieno[2,3-d]pyrimidin-4-yl)-2,7-diazaspiro[4.4]nonan-2-yl)methyl)phenyl)acetamide

To a solution of 3-((7-(6-(2,2,2-trifluoroethyl)thieno[2,3-d]pyrimidin-4-yl)-2,7-diazaspiro[4.4] nonan-2-yl)-methyl)aniline (40 mg, 0.089 mmol) in DCM (5 mL) were added acetyl chloride (8 mg, 0.098 mmol) and pyridine (15 mg, 0.08 mmol). Then the reaction was stirred at 25-30° C. for 2 h. LCMS showed about 93% of the title compound was detected. The mixture was concentrated and purified by preparative RP-HPLC method A to afford the title compound (11.1 mg); LCMS method C: Rₜ=0.64 min; (M+H)⁺=490.1; ¹H NMR (MeOD-d4): δ8.46 (s, 1H), 7.94 (s, 1H), 7.72 (s, 1H), 7.30-7.47 (m, 2H), 7.27-7.41 (m, 1H) 4.44 (s, 2H), 3.93-4.04 (m, 6H), 3.60-3.66 (m, 4H), 2.78 (s, 4H), 2.14 (s, 3H); ¹⁹F NMR (MeOD-d4): δ −67.657, −77.089.

### Example 53

### methyl (3-((7-(6-(2,2,2-trifluoroethyl)thieno[2,3-d]pyrimidin-4-yl)-2,7-diazaspiro[4.4]nonan-2-yl)methyl)phenyl)carbamate

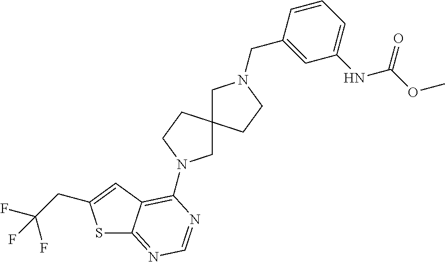 [SMILES: COC(=O)Nc1cccc(CN2CCC3(CCN(c4ncnc5sc(CC(F)(F)F)cc45)C3)C2)c1]

The title compound was prepared using procedures analogous to those described in Example 52. LCMS method C: Rₜ=0.66 min; (M+H)⁺=506.1; ¹H NMR (MeOD-d4): δ 8.50 (s, 1H), 7.74-7.78 (m, 2H), 7.39-7.40 (m, 2H), 7.20-7.21 (m, 1H), 4.43 (s, 2H) 3.92-4.06 (m, 6H), 3.75 (s, 3H), 3.56-3.69 (m, 4H), 3.49 (s, 3H), 2.29 (s, 4H); ¹⁹F NMR (MeOD-d4): δ −67.648, −77.163.

### Example 54

### N-(3-methyl-4-((7-(6-(2,2,2-trifluoroethyl)thieno[2,3-d]pyrimidin-4-yl)-2,7-diazaspiro[4.4]nonan-2-yl)methyl)phenyl)methanesulfonamide

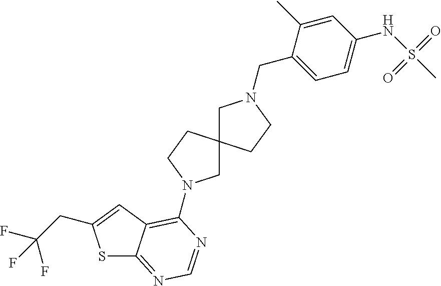 [SMILES: Cc1cc(NS(C)(=O)=O)ccc1CN1CCC2(CCN(c3ncnc4sc(CC(F)(F)F)cc34)C2)C1]

The title compound was prepared using procedures analogous to those described in Example 52. LCMS method C: Rₜ=0.656 min; (M+H)⁺=540.2; ¹H NMR (MeOD-d4): δ 8.40 (s, 1H), 7.67 (s, 1H), 7.47 (d, J=8.0 Hz, 1H), 7.14-7.27 (m, 2H), 4.49 (s, 2H), 3.84-4.10 (m, 6H), 3.37-3.80 (m, 4H), 3.00 (s, 3H), 2.47 (s, 3H), 2.03-2.39 (m, 4H); ¹⁹F NMR (MeOD-d4): δ −67.69, −77.12.

### Example 55

### piperidin-3-yl(7-(6-(2,2,2-trifluoroethyl)thieno[2,3-d]pyrimidin-4-yl)-2,7-diazaspiro[4.4]nonan-2-yl)methanone

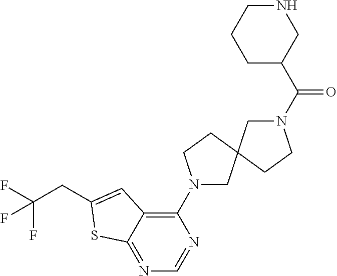 [SMILES: O=C(C1CCCNC1)N1CCC2(CCN(c3ncnc4sc(CC(F)(F)F)cc34)C2)C1]

### Step 1. tert-butyl 3-(7-(6-(2,2,2-trifluoroethyl)thieno[2,3-d]pyrimidin-4-yl)-2,7-diazaspiro[4.4]nonane-2-carbonyl)piperidine-1-carboxylate

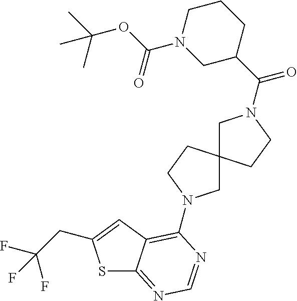 [SMILES: CC(C)(C)OC(=O)N1CCCC(C(=O)N2CCC3(CCN(c4ncnc5sc(CC(F)(F)F)cc45)C3)C2)C1]

To a solution of 1-(tert-butoxycarbonyl)piperidine-3-carboxylic acid (34 mg, 0.15 mmol), HATU (16 mg, 0.15 mmol) and DIEA (77 mg, 0.60 mmol) in DMF (3 mL) was added a solution of 4-(2,7-diazaspiro[4.4]nonan-2-yl)-6-(2,2,2-trifluoroethyl)thieno[2,3-d]pyrimidine (see Example 28, Step 2) (50 mg, 0.15 mmol) in DMF (2 mL). The mixture was stirred at 26-30° C. for 16 h. To the reaction mixture was added water (50 mL) and the resulting mixture was extracted with EtOAc (2×50 mL). The organic layers were dried over anhydrous Na₂SO₄, filtered and concentrated in vacuo to give crude tert-butyl 3-(7-(6-(2,2,2-trifluoroethyl)thieno[2,3-d]pyrimidin-4-yl)-2,7-diazaspiro[4.4]nonane-2-carbonyl)piperidine-1-carboxylate (113 mg), which was used in the next step directly without purification. LCMS method C: Rₜ=0.78 min; (M+H)⁺=554.1.

### Step 2. piperidin-3-yl(7-(6-(2,2,2-trifluoroethyl)thieno[2,3-d]pyrimidin-4-yl)-2,7-diazaspiro[4.4]nonan-2-yl)methanone

To a solution of tert-butyl 3-(7-(6-(2,2,2-trifluoroethyl)thieno[2,3-d]pyrimidin-4-yl)-2,7diazaspiro[4.4] nonane-2-carbonyl)piperidine-1-carboxylate (113 mg, 0.20 mmol, crude) in CH₂Cl₂ (4 mL) was added trifluoroacetic acid (2 mL). The mixture was stirred at 23-28° C. for 16 h. LCMS showed the starting material was consumed completely. The reaction mixture was concentrated in vacuo. The residue was neutralized with NH₃—H₂O (pH=8) and purified by preparative RP-HPLC method A to give the title compound as a transparent solid (50.7 mg). LCMS method C: Rₜ=0.62 min; (M+H)⁺=454.1; ¹H NMR (MeOD-d4) δ 8.48 (s, 1H), 7.80 (s, 1H), 4.05-4.15 (m, 2H), 3.96 (m, 4H), 3.46-3.84 (m, 4H), 3.18-3.30 (m, 3H), 2.99-3.17 (m, 2H), 2.12-2.32 (m, 3H), 1.89-2.12 (m, 3H), 1.70-1.89 (m, 2H); ¹⁹F NMR (MeOD-d4) δ −67.646, −77.068.

### Example 56

### piperidin-2-yl(7-(6-(2,2,2-trifluoroethyl)thieno[2,3-d]pyrimidin-4-yl)-2,7-diazaspiro[4.4]nonan-2-yl)methanone

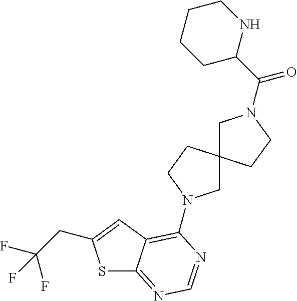 [SMILES: O=C(C1CCCCN1)N1CCC2(CCN(c3ncnc4sc(CC(F)(F)F)cc34)C2)C1]

The title compound was prepared using procedures analogous to those described in Example 55. In step 1, 1-(tert-butoxycarbonyl)piperidine-2-carboxylic acid (CAS registry number) was utilized. LCMS method C: Rₜ=0.62 min; (M+H)⁺=454.1; ¹H NMR (CD₃OD): δ 8.48 (d, 1H), 7.73-7.84 (d, 1H), 4.05-4.26 (m, 3H), 3.96 (m, 4H), 3.35-3.86 (m, 5H), 2.95-3.14 (m, 1H), 2.01-2.30 (m, 5H), 1.82-2.01 (m, 2H), 1.57-1.81 (m, 3H); ¹⁹F NMR (CD₃OD): δ−67.668, −77.106.

### Example 57

### 4-(7-prolyl-2,7-diazaspiro[4.4]nonan-2-yl)-6-(2,2,2-trifluoroethyl)thieno[2,3-d]pyrimidine

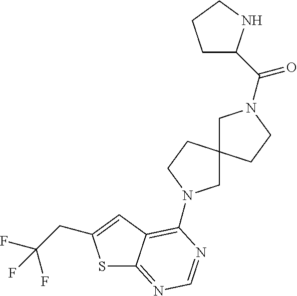 [SMILES: O=C(C1CCCN1)N1CCC2(CCN(c3ncnc4sc(CC(F)(F)F)cc34)C2)C1]

The title compound was prepared using procedures analogous to those described in Example 55. In step 1, 1-(tert-butoxycarbonyl)pyrrolidine-2-carboxylic acid (CAS registry number 59433-50-0) was utilized. LCMS method C: Rₜ=0.62 min; (M+H)⁺=440.5; ¹H NMR (CD₃OD): δ 8.50 (s, 1H), 7.80 (s, 1H), 4.47-4.61 (m, 1H), 3.86-4.32 (m, 6H), 3.60-3.85 (m, 3H), 3.49-3.60 (m, 1H), 3.32-3.48 (m, 2H), 2.42-2.62 (m, 1H), 1.90-2.30 (m, 7H); ¹⁹F NMR (CD₃OD): δ −67.668, −77.175.

### Example 58

### 2-Amino-2-methyl-1-(7-(6-(2,2,2-trifluoroethyl)thieno[2,3-d]pyrimidin-4-yl)-2,7-diazaspiro[4.4]nonan-2-yl)propan-1-one

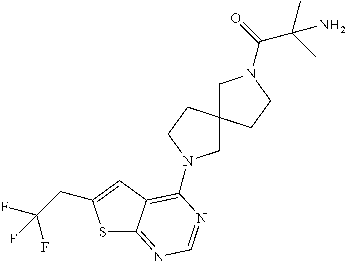 [SMILES: CC(C)(N)C(=O)N1CCC2(CCN(c3ncnc4sc(CC(F)(F)F)cc34)C2)C1]

The title compound was prepared using procedures analogous to those described in Example 55. In step 1, 2-((tert-butoxycarbonyl)amino)-2-methylpropanoic acid (CAS registry number 30992-29-1) was utilized. The title compound was isolated as a TFA salt. LCMS method B: Rₜ=0.90 min; (M+H)⁺=428.6; ¹H NMR (MeOH-d4): δ 8.39 (s, 1H), 7.72 (s, 1H), 4.09 (m, 2H), 3.92 (q, J=10.4 Hz, 2H), 3.88 (m, 3H), 3.71 (m, 3H), 2.17 (m, 4H), 1.66 (s, 6H). ¹⁹F NMR (MeOH-d4): δ −67.739 (t, J=10.4 Hz).

### Example 59

### 2-benzyl-7-(6-(2,2,2-trifluoroethyl)thieno[2,3-d]pyrimidin-4-yl)-2,7-diazaspiro[4.4]nonan-1-one

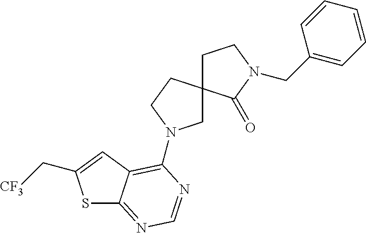 [SMILES: O=C1N(Cc2ccccc2)CCC12CCN(c1ncnc3sc(CC(F)(F)F)cc13)C2]

### Step 1: 7-(6-(2,2,2-trifluoroethyl)thieno[2,3-d]pyrimidin-4-yl)-2,7-diazaspiro[4.4]nonan-1-one

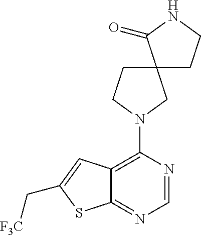 [SMILES: O=C1NCCC12CCN(c1ncnc3sc(CC(F)(F)F)cc13)C2]

To a solution of 4-chloro-6-(2,2,2-trifluoroethyl)thieno[2,3-d]pyrimidine (0.11 g, 0.44 mmol) in THF (1.5 mL) was added 2,7-diazaspiro[4.4]nonan-1-one hydrogen chloride salt (85 mg, 0.48 mmol), and Et₃N (0.2 mL). The resulting mixture was heated in a CEM microwave reactor at 110° C. for 80 min. The reaction was done by LCMS, reaction mixture was filtered and the solid was washed with EtOAc, combined filtration was evaporated under vacuum to give crude 7-(6-(2,2,2-trifluoroethyl)thieno[2,3-d]pyrimidin-4-yl)-2,7-diazaspiro[4.4]nonan-1-one (201 mg), which was used in the next step without purification. LCMS method B: Rₜ=1.18 min; (M+H)⁺=357.5.

### Step 2: 2-benzyl-7-(6-(2,2,2-trifluoroethyl)thieno[2,3-d]pyrimidin-4-yl)-2,7-diazaspiro[4.4]-nonan-1-one

To a solution of 7-(6-(2,2,2-trifluoroethyl)thieno[2,3-d]pyrimidin-4-yl)-2,7-diazaspiro[4.4]nonan-1-one (33 mg, 0.07 mmol) in DMF at 0° C., was added NaH (5 mg, excess) in DMF (0.2 mL). Benzyl bromide (13.2 mg, 0.08 mmol) was added after the mixture was stirred for 5 min. The reaction went to completion within 15 min as indicated by LCMS. The reaction was quenched with ice water and extracted with DCM. Combined organic layers were concentrated, and purified by HPLC to give the title compound as a TFA salt. LCMS method B: Rₜ=1.45 min; (M+H)⁺=427.5; ¹H NMR (methanol-d4): δ 8.54, 8.50 (two s, 1H), 7.84, 7.72 (two s, 1H), 7.24-7.38 (m, 5H), 4.48 (dd, 2H), 3.90-4.38 (m, 4H), 3.96 (t, J=10.4 Hz, 2H), 3.38 (m, 2H), 2.40 (m, 2H), 2.20 (m, 2H). ¹⁹F NMR (methanol-d₄): δ −67.694 (t, J=10.4 Hz).

### Example 60

### ((1s,4s)-4-methoxycyclohexyl)(6-(6-(2,2,2-trifluoroethyl)thieno[2,3-d]pyrimidin-4-yl)-2,6-diazaspiro[3.3]heptan-2-yl)methanone and ((1r,4r)-4-methoxycyclohexyl)(6-(6-(2,2,2-trifluoroethyl)thieno[2,3-d]pyrimidin-4-yl)-2,6-diazaspiro[3.3]heptan-2-yl)methanone

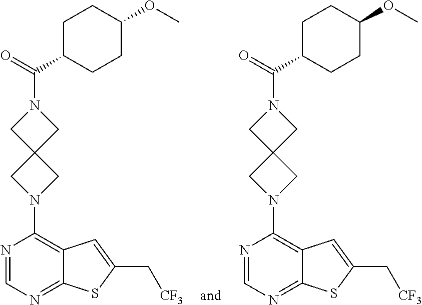 [SMILES: CO[C@H]1CC[C@@H](C(=O)N2CC3(CN(c4ncnc5sc(CC(F)(F)F)cc45)C3)C2)CC1 ; CO[C@H]1CC[C@H](C(=O)N2CC3(CN(c4ncnc5sc(CC(F)(F)F)cc45)C3)C2)CC1]

### Step 1: tert-butyl 6-(6-(2,2,2-trifluoroethyl)thieno[2,3-d]pyrimidin-4-yl)-2,6-diazaspiro[3.3]heptane-2-carboxylate

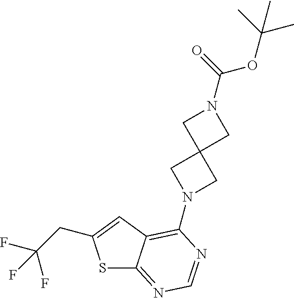 [SMILES: CC(C)(C)OC(=O)N1CC2(C1)CN(c1ncnc3sc(CC(F)(F)F)cc13)C2]

To a solution of 4-chloro-6-(2,2,2-trifluoroethyl)thieno[2,3-d]pyrimidine (1.0 g, 3.96 mmol) in CH₃CN (30 mL) was added tert-butyl 2,6-diazaspiro[3.3]heptane-2-carboxylate (929 mg, 3.96 mmol) and Na₂CO₃ (1.0 g, 9.90 mmol). The resulting mixture was stirred at 90° C. for about 4 h. The reaction mixture was filtered. The filtrate was concentrated in vacuo. The residue was purified by column chromatography on silica gel (petroleum ether:EtOAc=5:1 to EtOAc) to give tert-butyl 6-(6-(2,2,2-trifluoroethyl)thieno[2,3-d]pyrimidin-4-yl)-2,6-diazaspiro[3.3]heptane-2-carboxylate (1.5 g). LCMS method C: Rₜ=0.77 min; (M+H)⁺=415.1

### Step 2: 4-(2,6-diazaspiro[3.3]heptan-2-yl)-6-(2,2,2-trifluoroethyl)thieno[2,3-d]pyrimidine

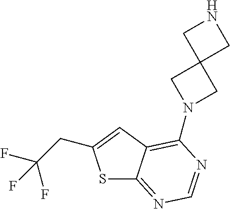 [SMILES: FC(F)(F)Cc1cc2c(N3CC4(CNC4)C3)ncnc2s1]

To a solution of 6-(6-(2,2,2-trifluoroethyl)thieno[2,3-d]pyrimidin-4-yl)-2,6-diazaspiro[3.3]heptane-2-carboxylate (1.58 g, 3.81 mmol) in CH₂Cl₂ (10 mL, anhydrous) was added TFA (3 mL). The resulting mixture was stirred at 23-34° C. for about 4 h. The reaction mixture was neturalized by NH₃.H₂O to pH=6.0-7.0. The aqueous layer was extracted with CH₂Cl₂ (2×30 mL). The organic layers were concentrated in vacuo to give 4-(2,6-diazaspiro [3.3]heptan-2-yl)-6-(2,2,2-trifluoroethyl)thieno[2,3-d]pyrimidine (1.1 g). LCMS method C: Rₜ=0.53 min; (M+H)⁺=315.1

### Step 3: ((1s,4s)-4-methoxycyclohexyl)(6-(6-(2,2,2-trifluoroethypthieno[2,3-d]pyrimidin-4-yl)-2,6-diazaspiro[3.3]heptan-2-yl)methanone) and ((1r,4r)-4-methoxycyclohexyl)(6-(6-(2,2,2-trifluoroethyl)thieno[2,3-d]pyrimidin-4-yl)-2,6-diazaspiro[3.3]heptan-2-yl)methanone

To a solution of 4-methoxycyclohexanecarboxylic acid (101 mg, 0.64 mmol) in DMF (10 mL, anhydrous) was added DIEA (486 mg, 2.56 mmol) and HATU (243 mg, 0.64 mmol). After stirring for 10 min, a solution of 4-(2,6-diazaspiro[3.3]heptan-2-yl)-6-(2,2,2-trifluoroethyl)thieno[2,3-d]pyrimidine (200 mg, 0.64 mmol) in DMF (5 mL, anhydrous) was added via syringe. The resulting mixture was stirred at 23-29° C. for 4 h. The reaction mixture was diluted with water (30 mL) and extracted with EtOAc (2×30 mL). The organic layers were concentrated in vacuo. The residue was purified by preparative-TLC (CH₂Cl₂:MeOH=15:1) to give the product (160 mg, 55%, a mixture of two isomers) as a yellow oil.

An amount of 60 mg of this oil was purified by acidic preparative RP-HPLC method A and dried by lyophilization to give Isomer 1 and Isomer 2 (as TFA salts) both as colorless oil.

Isomer 1 (Example 60a): LCMS method C: Rₜ=0.69 min; (M+H)⁺=455.1; ¹H NMR (Methanol-d4): δ 8.43 (s, 1H), 7.49 (s, 1H), 4.72 (s, 4H), 4.53 (s, 2H), 4.24 (s, 2H), 3.96 (q, J=21.2, 10.8 Hz, 2H), 3.37 (s, 3H), 3.16-3.22 (m, 1H), 2.17-2.27 (m, 1H), 2.12-2.16 (m, 2H), 1.80-1.85 (m, 2H), 1.44-1.57 (m, 2H), 1.16-1.28 (m, 2H); ¹⁹F NMR (Methanol-d4): δ −67.691, −77.311.

Isomer 2 (Example 60b): LCMS method C: Rₜ=0.71 min; (M+H)⁺=455.1; ¹H NMR (Methanol-d4): δ 8.46 (s, 1H), 7.52 (s, 1H), 4.75 (s, 4H), 4.53 (s, 2H), 4.24 (s, 2H), 3.98 (q, J=20.8, 10.0 Hz, 2H), 3.48 (s, 3H), 3.14-3.20 (m, 1H), 2.30-2.38 (m, 1H), 2.00-2.10 (m, 2H), 1.72-1.86 (m, 2H), 1.45-1.57 (m, 4H). ¹⁹F NMR (Methanol-d₄): δ −67.668, −77.319.

### Example 61

### 2-(4-((methylsulfonyl)methyl)phenyl)-1-(6-(6-(2,2,2-trifluoroethyl)thieno[2,3-d]pyrimidin-4-yl)-2,6-diazaspiro[3.3]heptan-2-yl)ethan-1-one

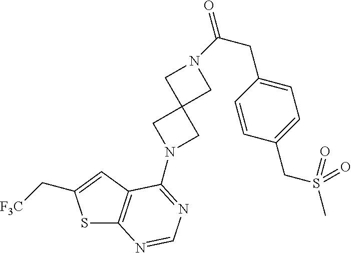 [SMILES: CS(=O)(=O)Cc1ccc(CC(=O)N2CC3(C2)CN(c2ncnc4sc(CC(F)(F)F)cc24)C3)cc1]

The title compound was prepared using procedures analogous to those described for Example 60. The title compound was isolated as a TFA salt. In step 3, 2-(4-((methylsulfonyl)methyl)phenyl)acetic acid (CAS registry number 1598899-63-8) was utilized. LCMS method B: Rₜ=1.08 min; (M+H)⁺=525.4.

### Example 62

### 2-(4-(methylsulfonyl)phenyl)-1-(6-(6-(2,2,2-trifluoroethyl)thieno[2,3-d]pyrimidin-4-yl)-2,6-diazaspiro[3.3]heptan-2-yl)ethan-1-one

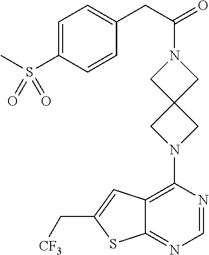 [SMILES: CS(=O)(=O)c1ccc(CC(=O)N2CC3(C2)CN(c2ncnc4sc(CC(F)(F)F)cc24)C3)cc1]

The title compound was prepared using procedures analogous to those described for Example 60. In step 3, 2-(4-((methylsulfonyl))phenyl)acetic acid was utilized. The title compound was isolated as a TFA salt. LCMS method B: Rₜ=1.20 min; (M+H)⁺=511.5; ¹H NMR (methanol-d4): δ 8.42 (s, 1H), 7.92 (d, J=8 Hz, 2H), 7.58 (d, J=8Hz, 2H), 7.46 (s, 1H), 4.74 (m, 4H), 4.56 (s, 2H), 4.26 (s, 2H), 3.92 (q, J=10.4 Hz, 2H), 3.64 (s, 2H), 3.08 (s, 3H); ¹⁹F NMR (methanol-d4): δ −67.718 (t, J=10.4 Hz).

### Example 63

### 4-(6-((3,3-difluorocyclohexyl)methyl)-2,6-diazaspiro[3.3]heptan-2-yl)-6-(2,2,2-trifluoroethyl)thieno[2,3-d]pyrimidine

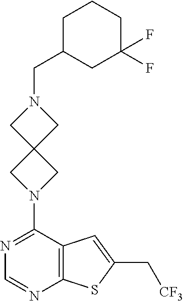 [SMILES: FC(F)(F)Cc1cc2c(N3CC4(CN(CC5CCCC(F)(F)C5)C4)C3)ncnc2s1]

The title compound was prepared using procedures analogous to those described in Example 11. The compound 4-(2,6-diazaspiro[3.3]heptan-2-yl)-6-(2,2,2-trifluoroethyl)thieno[2,3-d]pyrimidine (see Example 60, Step 2) and 3,3-difluorocyclohexane-1-carbaldehyde were utilized to yield the title compound. LCMS method C: Rₜ=0.65 min; (M+H)⁺=447.1; ¹H NMR (methanol-d4): δ 8.43 (s, 1H), 7.43 (s, 1H), 4.40-4.75 (m, 8H), 3.95 (q, J=10.4 Hz, 2H), 3.25 (m, 2H), 1.50-2.20 (m, 8H), 1.13-1.20 (m, 1H); ¹⁹F NMR (methanol-d4): δ −67.90˜−67.23, −77.14, −90.79˜−90.15, −101.75˜−101.11.

### Example 64

### 4-(6-((4,4-dimethylcyclohexyl)methyl)-2,6-diazaspiro[3.3]heptan-2-yl)-6-(2,2,2-trifluoroethyl)thieno[2,3-d]pyrimidine

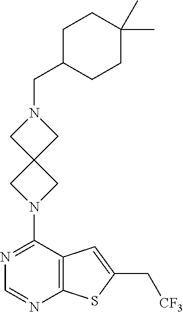 [SMILES: CC1(C)CCC(CN2CC3(C2)CN(c2ncnc4sc(CC(F)(F)F)cc24)C3)CC1]

The title compound was prepared using procedures analogous to those described in Example 11. The compound 4-(2,6-diazaspiro[3.3]heptan-2-yl)-6-(2,2,2-trifluoroethyl)thieno[2,3-d]pyrimidine and 4,4-dimethylcyclohexane-1-carbaldehyde (CAS registry number 394734-96-4) were were utilized to yield the title compound. LCMS method C: Rₜ=0.72 min; (M+H)⁺=439.1; ¹H NMR: (MeOH-d4): δ 8.37 (s, 1H), 7.38 (s, 1H), 4.47-4.83 (m, 6H), 4.29-4.46 (m, 2H), 3.92 (q, J=10.4 Hz, 2H), 3.15 (d, J=6.8 Hz, 2H), 1.52-1.70 (m, 3H), 1.43-1.46 (m, 2H), 1.15-1.33 (m, 4H), 0.92 (d, J=5.6 Hz, 6H); ¹⁹F NMR: (MeOH-d4): δ −67.668, −76.977.

### Example 65

### 4-(6-((3,3-dimethylcyclohexyl)methyl)-2,6-diazaspiro[3.3]heptan-2-yl)-6-(2,2,2-trifluoroethyl)thieno[2,3-d]pyrimidine

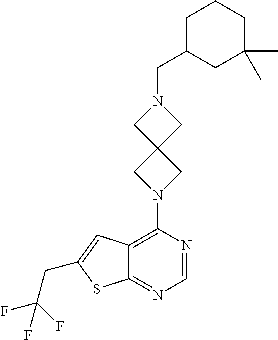 [SMILES: CC1(C)CCCC(CN2CC3(C2)CN(c2ncnc4sc(CC(F)(F)F)cc24)C3)C1]

The title compound was prepared using procedures analogous to those described in Example 11. The compound 4-(2,6-diazaspiro[3.3]heptan-2-yl)-6-(2,2,2-trifluoroethyl)thieno[2,3-d]pyrimidine and 3,3-dimethylcyclohexane-1-carbaldehyde (CAS registry number 99017-89-7) were utilized to yield the title compound. LCMS method C: Rₜ=0.72 min; (M+H)⁺=439.1; ¹H NMR: (methanol-d4): δ 8.36 (s, 1H), 7.38 (s, 1H), 4.45-4.82 (m, 6H), 4.23-4.44 (m, 2H), 3.77-4.00 (m, 2H), 3.00-3.15 (m, 2H), 1.69-1.92 (m, 2H), 1.31-1.68 (m, 4H), 1.08-1.20 (m, 1H), 0.79-1.02 (m, 8H). ¹⁹F NMR: (methanol-d4): δ −67.668, −76.954.

### Example 66

### 4-(6-((tetrahydro-2H-pyran-4-yl)methyl)-2,6-diazaspiro[3.3]heptan-2-yl)-6-(2,2,2-trifluoroethyl)thieno[2,3-d]pyrimidine

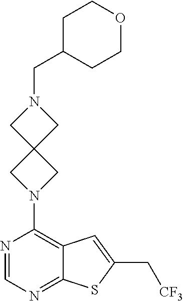 [SMILES: FC(F)(F)Cc1cc2c(N3CC4(CN(CC5CCOCC5)C4)C3)ncnc2s1]

The title compound was prepared using procedures analogous to those described in Example 11. The compound 4-(2,6-diazaspiro[3.3]heptan-2-yl)-6-(2,2,2-trifluoroethyl)thieno[2,3-d]pyrimidine and tetrahydro-2H-pyran-4-carbaldehyde (CAS registry number 50675-18-8) were utilized to give the title compound. The title compound was isolated as a TFA salt. LCMS method B: Rₜ=0.92 min; (M+H)⁺=397.5; ¹H NMR (methanol-d4): δ 8.42 (s, 1H), 7.42 (s, 1H), 4.80 (m, 2H), 4.68 (m, 2H), 4.60 (m, 2H), 4.42 (m, 2H), 3.96 (m, 4H), 3.42 (t, J=10 Hz, 2H), 3.18 (d, J=8 Hz, 2H), 1.96 (m, 1H), 1.64 (m, 2H), 1.38 (m, 2H). ¹⁹F NMR (MeOH-d4): δ −67.711 (t, J=10.4 Hz).

### Example 67

### 4-methyl-5-((6-(6-(2,2,2-trifluoroethyl)thieno[2,3-d]pyrimidin-4-yl)-2,6-diazaspiro[3.3]heptan-2-yl)methyl)-1H-indole-2-carbonitrile

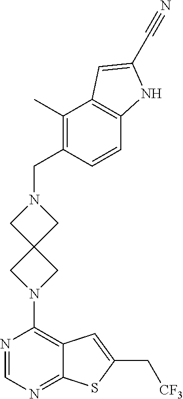 [SMILES: Cc1c(CN2CC3(C2)CN(c2ncnc4sc(CC(F)(F)F)cc24)C3)ccc2[nH]c(C#N)cc12]

The title compound was prepared using procedures analogous to those described in Example 11. The compound 4-(2,6-diazaspiro[3.3]heptan-2-yl)-6-(2,2,2-trifluoroethyl)thieno[2,3-d]pyrimidine and 5-formyl-4-methyl-1H-indole-2-carbonitrile were utilized to yield the title compound. The title compound was isolated as a TFA salt. LCMS method B: Rₜ=0.96 min; (M+H)⁺=483.5.

### Example 68

### 4-(6-(cyclopentylmethyl)-2,6-diazaspiro[3.3]heptan-2-yl)-6-(2,2,2-trifluoroethyl)thieno[2,3-d]pyrimidine

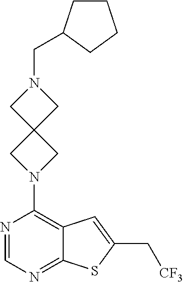 [SMILES: FC(F)(F)Cc1cc2c(N3CC4(CN(CC5CCCC5)C4)C3)ncnc2s1]

The title compound was prepared using procedures analogous to those described in Example 11. The compounds 4-(2,6-diazaspiro[3.3]heptan-2-yl)-6-(2,2,2-trifluoroethyl)thieno[2,3-d]pyrimidine and cyclopentanecarbaldehyde (CAS registry number 872-53-7) were utilized to yield the title compound. The title compound was isolated as a TFA salt. LCMS method B: Rₜ=0.92 min; (M+H)⁺=397.5; ¹H NMR (MeOH-d4): δ 8.38 (s, 1H), 7.39 (s, 1H), 4.74 (m, 2H), 4.64 (m, 2H), 4.53 (m, 2H), 4.38 (m, 2H), 3.92 (q, J=10.4 Hz, 2H), 3.22 (d, J=8 Hz, 2H), 2.09 (m, 1H), 1.87 (m, 2H), 1.58-1.78 (m, 4H), 1.26 (m, 2H). ¹⁹F NMR (MeOH-d4): δ −67.708 (t, J=10.4 Hz).

### Example 69

### 4-(6-(cyclohexylmethyl)-2,6-diazaspiro[3.3]heptan-2-yl)-6-(2,2,2-trifluoroethyl)thieno[2,3-d]pyrimidine

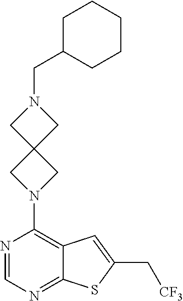 [SMILES: FC(F)(F)Cc1cc2c(N3CC4(CN(CC5CCCCC5)C4)C3)ncnc2s1]

The title compound was prepared using procedures analogous to those described in Example 11. The compounds 4-(2,6-diazaspiro[3.3]heptan-2-yl)-6-(2,2,2-trifluoroethyl)thieno[2,3-d]pyrimidine and cyclohexanecarbaldehyde (CAS registry number 2043-61-0) were utilized to yield the title compound. The title compound was isolated as a TFA salt. LCMS method B: Rₜ=0.98 min; (M+H)⁺=411.5; ¹H NMR (MeOH-d4): δ 8.39 (s, 1H), 7.39 (s, 1H), 4.74 (m, 2H), 4.63 (m, 2H), 4.54 (m, 2H), 4.36 (m, 2H), 3.93 (q, J=10.6 Hz, 2H), 3.11 (d, J=8 Hz, 2H), 1.60-1.82 (m, 6H), 1.18-1.38 (m, 3H), 0.98-1.10 (m, 2H). ¹⁹F NMR (MeOH-d4): δ −67.718 (t, J=10.6 Hz).

### Example 70

### 4-(6-((4,4-difluorocyclohexyl)methyl)-2,6-diazaspiro[3.3]heptan-2-yl)-6-(2,2,2-trifluoroethyl)thieno[2,3-d]pyrimidine

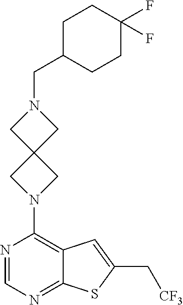 [SMILES: FC(F)(F)Cc1cc2c(N3CC4(CN(CC5CCC(F)(F)CC5)C4)C3)ncnc2s1]

The title compound was prepared using procedures analogous to those described in Example 11. The compounds 4-(2,6-diazaspiro[3.3]heptan-2-yl)-6-(2,2,2-trifluoroethyl)thieno[2,3-d]pyrimidine and 4,4-difluorocyclohexane-1-carbaldehyde were utilized to yield the title compound. The title compound was isolated as a TFA salt. LCMS method B: Rₜ=0.96 min; (M+H)⁺=447.6.

### Example 71

### N-((1r,4r)-4-((6-(6-(2,2,2-trifluoroethyl)thieno[2,3-d]pyrimidin-4-yl)-2,6-diazaspiro[3.3]heptan-2-yl)methyl)cyclohexyl)ethanesulfonamide

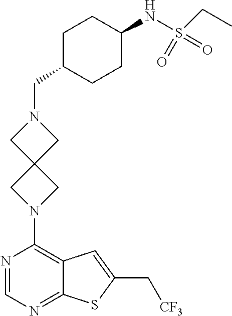 [SMILES: CCS(=O)(=O)N[C@H]1CC[C@H](CN2CC3(C2)CN(c2ncnc4sc(CC(F)(F)F)cc24)C3)CC1]

The title compound was synthesized from 4-(2,6-diazaspiro[3.3]heptan-2-yl)-6-(2,2,2-trifluoroethyl)thieno[2,3-d]pyrimidine and tert-butyl ((1r,4r)-4-(hydroxymethyl)cyclohexyl)carbamate using procedures analogous to those described in Example 46, Steps 1 through 4. In Step 4, ethanesulfonyl chloride was used instead of methyl carbonochloridate. The title compound was isolated as a TFA salt. LCMS method B: Rₜ=1.02 min; (M+H)⁺=518.6.

### Example 72

### N-((1r,4r)-4-((6-(6-(2,2,2-trifluoroethyl)thieno[2,3-d]pyrimidin-4-yl)-2,6-diazaspiro[3.3]heptan-2-yl)methyl)cyclohexyl)cyclopropanesulfonamide

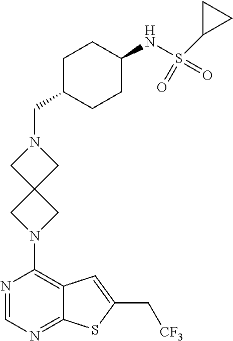 [SMILES: O=S(=O)(N[C@H]1CC[C@H](CN2CC3(C2)CN(c2ncnc4sc(CC(F)(F)F)cc24)C3)CC1)C1CC1]

The title compound was synthesized from 4-(2,6-diazaspiro[3.3]heptan-2-yl)-6-(2,2,2-trifluoroethyl)thieno[2,3-d]pyrimidine and tert-butyl ((1r,4r)-4-(hydroxymethyl)cyclohexyl)carbamate using procedures analogous to those described in Example 46, Steps 1 through 4. In Step 4, cyclopropanesulfonyl chloride was used instead of methyl carbonochloridate. The title compound was isolated as a TFA salt. LCMS method B: Rₜ=1.00 min; (M+H)⁺=530.6.

### Example 73

### 4-(6-(oxetan-3-ylmethyl)-2,6-diazaspiro[3.3]heptan-2-yl)-6-(2,2,2-trifluoroethyl)thieno[2,3-d]pyrimidine

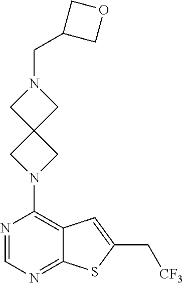 [SMILES: FC(F)(F)Cc1cc2c(N3CC4(CN(CC5COC5)C4)C3)ncnc2s1]

### Step 1: oxetan-3-ylmethyl 4-methylbenzenesulfonate

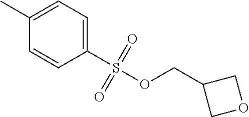 [SMILES: Cc1ccc(S(=O)(=O)OCC2COC2)cc1]

To a solution of oxetan-3-ylmethanol (CAS registry number 6246-06-6; 100 mg, 1.14 mmol) in DCM (5 mL) were added TsCl (260 mg, 1.36 mmol) and pyridine (180 mg, 2.27 mmol). Then the reaction was stirred at 23-29° C. for 16 h. TLC (PE:EA=1:1) confirmed that starting material was consumed and a new product was formed. The mixture was concentrated and purified by chromatography column on silica gel, eluting with PE:EA=5:1, to afford oxetan-3-ylmethyl 4-methylbenzenesulfonate (100 mg). ¹H NMR (CDCl₃) δ 7.81 (d, J=8.0 Hz, 2H), 7.37 (d, J=8.0 Hz, 2H), 4.73-4.77 (t, J=6.8 Hz, 2H), 4.32-4.35 (t, J=5.6 Hz, 2H), 4.26 (d, J=7.2 Hz, 2H), 3.26-3.33 (m, 1H), 2.47 (s, 3H).

### Step 2: 4-(6-(oxetan-3-ylmethyl)-2,6-diazaspiro[3.3]heptan-2-yl)-6-(2,2,2-trifluoroethyl)thieno[2,3-d]pyrimidine

To a solution of 4-(2,6-diazaspiro[3.3]heptan-2-yl)-6-(2,2,2-trifluoroethyl)thieno[2,3-d]pyrimidine (40 mg, 0.14 mmol) in DMF (3 mL) were added oxetan-3-ylmethyl 4-methylbenzenesulfonate (47 mg, 0.19 mmol) and TEA (40 mg, 0.39 mmol). Then the reaction was heated at 100° C. for 16 h. The mixture was concentrated and purified by preparative RP-LCMS method D to give the title compound (8 mg); LCMS method C: Rₜ=1.1 min; (M+H)⁺=385.0; ¹H NMR (methanol-d4) δ 8.27 (s, 1H), 7.36 (s, 1H), 4.76-4.80 (m, 2H), 4.43-4.49 (m, 4H), 4.41 (t, J=7.6 Hz, 2H), 3.84-3.92 (q, J=6.0 Hz, 2H), 3.48 (s, 4H), 3.03-3.10 (m, 2H), 2.81 (d, J=7.2 Hz, 1H); ¹⁹F NMR (methanol-d4) δ −67.174.

### Example 74

### 4-(6-((3,3-difluorocyclobutyl)methyl)-2,6-diazaspiro[3.3]heptan-2-yl)-6-(2,2,2-trifluoroethyl)thieno[2,3-d]pyrimidine

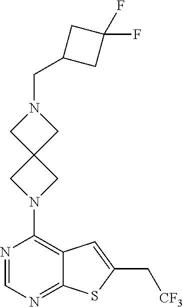 [SMILES: FC(F)(F)Cc1cc2c(N3CC4(CN(CC5CC(F)(F)C5)C4)C3)ncnc2s1]

To a mixture of 4-(2,6-diazaspiro[3.3]heptan-2-yl)-6-(2,2,2-trifluoroethyl)thieno[2,3-d]pyrimidine (50 mg, 0.16 mmol) and (3,3-difluorocyclobutyl)methyl 4-methylbenzenesulfonate (88 mg, 0.32 mmol) in DMF (3 mL) was added Et₃N (80.50 mg, 0.80 mmol) under a nitrogen atmosphere. The reaction mixture was stirred at 100° C. for 16 h. Water (80 mL) was added and the resulting mixture was extracted with EtOAc (3×80 mL). The organic layers were dried over anhydrous Na₂SO₄, filtered and concentrated in vacuo. The residue was purified by preparative RP-HPLC method D and dried by lyophilization to give the title compound (19 mg). LCMS method C: Rₜ=0.63 min; (M+H)⁺=419.1; ¹H NMR (DMSO-d6) δ 8.30 (s, 1H), 7.42 (s, 1H), 4.30-4.45 (m, 4H), 4.03 (q, J=11.2 Hz, 2H), 3.35-3.47 (m, 2H), 3.15-3.25 (m, 2H), 2.50-2.65 (m, 2H), 2.43 (d, J=7.2 Hz, 2H), 2.11-2.27 (m, 2H), 1.95-2.05 (m, 1H); ¹⁹F NMR (MeOH-d4) δ −67.732, −83.707˜−84.216, −96.620˜−97.129.

### Example 75

### 4-(6-((4-methoxycyclohexyl)methyl)-2,6-diazaspiro[3.3]heptan-2-yl)-6-(2,2,2-trifluoroethyl)thieno[2,3-d]pyrimidine

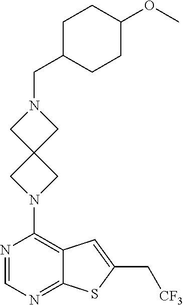 [SMILES: COC1CCC(CN2CC3(C2)CN(c2ncnc4sc(CC(F)(F)F)cc24)C3)CC1]

The title compound was prepared using procedures analogous to those described in Example 74, by reacting 4-(2,6-diazaspiro[3.3]heptan-2-yl)-6-(2,2,2-trifluoroethyl)thieno[2,3-d]pyrimidine with (4-methoxycyclohexyl)methyl 4-methylbenzenesulfonate to give a TFA salt of the title compound (21.0 mg, 23%). LCMS method E: Rₜ=0.84 min; (M+H)⁺=441.2; ¹H NMR (MeOH-d₄, 400 MHz): δ 8.39 (s, 1H), 7.40 (s, 1H), 4.33-4.80 (m, 8H), 3.94 (q, J=10.8 Hz, 2H), 3.50 (s, 1H), 3.32 (s, 3H), 3.15 (d, J=6.8 Hz, 2H), 1.92-2.00 (m, 2H), 1.65-1.75 (m, 1H), 1.46-1.57 (m, 4H), 1.29-1.46 (m, 2H).; ¹⁹F NMR (MeOH-d₄, 400 MHz): δ −67.70, −77.08.

### Example 76

### 4-((6-(6-(2,2,2-trifluoroethyl)thieno[2,3-d]pyrimidin-4-yl)-2,6-diazaspiro[3.3]heptan-2-yl)methyl)cyclohexan-1-ol

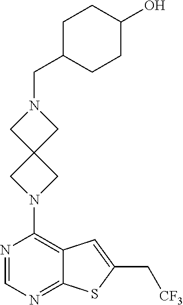 [SMILES: OC1CCC(CN2CC3(C2)CN(c2ncnc4sc(CC(F)(F)F)cc24)C3)CC1]

To a solution of 4-(6-((4-methoxycyclohexyl)methyl)-2,6-diazaspiro[3.3]heptan-2-yl)-6-(2,2,2-trifluoroethyl)thieno[2,3-d]pyrimidine (see Example 75) (10 mg, 0.023 mmol) in DCE (3 mL, anhydrous) was added TMSI (23 mg, 15 μL, 0.113 mmol) in one portion at 0-5° C. The resulting mixture was stirred at 27-35° C. under N₂ for 3 h. The reaction mixture was quenched with MeOH (1 mL) and concentrated in vacuo. The residue was purified by preparative RP-HPLC method A and dried by lyophilization to give the title compound (TFA salt) as a colorless oil (5.7 mg, 47%). LCMS method E: Rₜ=0.76; (M+H)⁺=472.1; ¹H NMR (MeOH-d₄, 400 MHz): δ 8.36 (s, 1H), 7.38 (s, 1H), 4.48-4.77 (m, 6H), 4.34-4.46 (m, 2H), 3.85-4.00 (m, 3H), 3.18 (d, J=7.2 Hz, 2H), 1.68-1.84 (m, 3H), 1.44-1.66 (m, 6H); ¹⁹F NMR (MeOH-d₄, 400 MHz): δ −67.69, −76.95.

### Example 77

### 3-amino-3-methyl-1-(6-(6-(2,2,2-trifluoroethyl)thieno[2,3-d]pyrimidin-4-yl)-2,6-diazaspiro[3.3]heptan-2-yl)butan-1-one

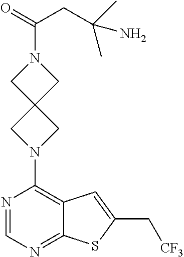 [SMILES: CC(C)(N)CC(=O)N1CC2(C1)CN(c1ncnc3sc(CC(F)(F)F)cc13)C2]

### Step 1. tert-butyl (2-methyl-4-oxo-4-(6-(6-(2,2,2-trifluoroethyl)thieno[2,3-d]pyrimidin-4-yl)-2,6-diazaspiro[3.3]heptan-2-yl)butan-2-yl)carbamate

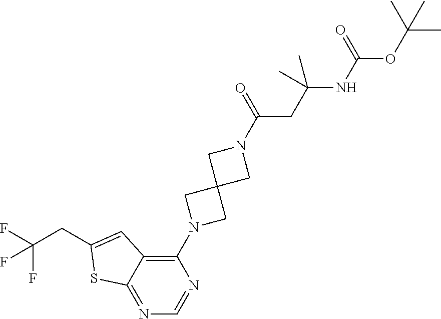 [SMILES: CC(C)(CC(=O)N1CC2(C1)CN(c1ncnc3sc(CC(F)(F)F)cc13)C2)NC(=O)OC(C)(C)C]

To a solution of 3-((tert-butoxycarbonyl)amino)-3-methylbutanoic acid (CAS registry number 129765-95-3; 35 mg, 0.16 mmol) in DMF (5 mL, anhydrous) was added DIEA (83 mg, 0.64 mmol), HATU (61 mg, 0.16 mmol), and 4-(2,6-diazaspiro[3.3]heptan-2-yl)-6-(2,2,2-trifluoroethyl)thieno[2,3-d]pyrimidine (50 mg, 0.16 mmol). The resulting mixture was stirred at 21-27° C. for 20 h. The reaction mixture was diluted with water (20 mL) and extracted with EtOAc (2×20 mL). The organic layers were washed with brine (3×30 mL), dried over anhydrous Na₂SO₄, filtered and concentrated in vacuo to give the desired product (65 mg, crude) as a yellow oil which was used in the next step directly. LCMS method C: Rₜ=0.76 min; (M+H)⁺=514.2.

### Step 2. 3-amino-3-methyl-1-(6-(6-(2,2,2-trifluoroethyl)thieno[2,3-d]pyrimidin-4-yl)-2,6-diazaspiro[3.3]heptan-2-yl)butan-1-one

To a solution of tert-butyl (2-methyl-4-oxo-4-(6-(6-(2,2,2-trifluoroethyl)thieno[2,3-d]pyrimidin-4-yl)-2,6-diazaspiro[3.3]heptan-2-yl)butan-2-yl)carbamate (60 mg, 0.12 mmol) in CH₂Cl₂ (2 mL, anhydrous) was added TFA (1 mL). The resulting mixture was stirred at 21-25° C. for 1 h. LCMS indicated the reaction went to completion. The reaction mixture was neutralized by NH₃.H₂O to pH=6-7. The mixture was filtered and concentrated in vacuo. The residue was purified by RP-HPLC method A and dried by lyophilization to give the title compound (80 mg). LCMS method C: Rₜ=0.59 min; (M+H)⁺=414.1; ¹H NMR (methanol-d4): δ 8.49 (s, 1H), 7.53 (s, 1H), 4.79 (s, 4H), 4.54 (s, 2H), 4.32 (s, 2H), 3.99 (q, J=10.4 Hz, 2H), 2.51 (s, 2H), 1.44 (s, 6H); ¹⁹FNMR (methanol-d4): δ −67.63, −77.11.

### Example 78

### piperidin-3-yl(6-(6-(2,2,2-trifluoroethyl)thieno[2,3-d]pyrimidin-4-yl)-2,6-diazaspiro[3.3]heptan-2-yl)methanone

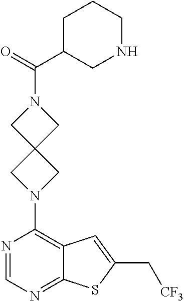 [SMILES: O=C(C1CCCNC1)N1CC2(C1)CN(c1ncnc3sc(CC(F)(F)F)cc13)C2]

The title compound was prepared using procedures analogous to those described in Example 77. The compound 1-(tert-butoxycarbonyl)piperidine-3-carboxylic acid (CAS registry number 84358-12-3) was utilized in Step 1. LCMS method C: Rₜ=0.59 min; (M+H)⁺=426.1; ¹H NMR: (methanol-d4): δ 8.47 (s, 1H), 7.51 (s, 1H), 4.76 (s, 4H), 4.52-4.60 (m, 2H), 4.24-4.34 (m, 2H), 3.98 (q, J=10.4 Hz, 2H), 3.17-3.32 (m, 3H), 3.07-3.17 (m, 1H), 2.80-2.91 (m, 1H), 1.91-2.05 (m, 2H), 1.73-1.89 (m, 2H); ¹⁹F NMR: (methanol-d4): δ −67.67, −77.18.

### Example 79

### piperidin-2-yl(6-(6-(2,2,2-trifluoroethyl)thieno[2,3-d]pyrimidin-4-yl)-2,6-diazaspiro[3.3]heptan-2-yl)methanone

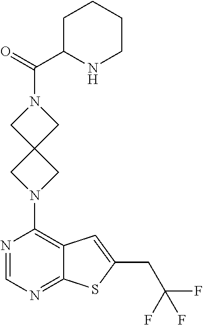 [SMILES: O=C(C1CCCCN1)N1CC2(C1)CN(c1ncnc3sc(CC(F)(F)F)cc13)C2]

The title compound was prepared using procedures analogous to those described in Example 77. The compound 1-(tert-butoxycarbonyl)piperidine-2-carboxylic acid (CAS registry number 98303-20-9) was utilized in Step 1. Yield: 24.3 mg. LCMS method C: Rₜ=0.60 min; (M+H)⁺=426.0; ¹H NMR (MeOH-d₄): δ 8.42 (s, 1H), 7.46 (s, 1H), 4.72 (m, 4H), 4.48-4.59 (m, 2H), 4.22-4.35 (m, 2H), 3.93 (m, 3H), 3.37 (d, J=12.8 Hz, 1H), 2.96-3.07 (m, 1H), 2.06-2.17 (m, 1H), 1.81-1.95 (m, 2H), 1.54-1.75 (m, 3H); ¹⁹F NMR (MeOH-d4): δ −67.684, −77.258.

### Example 80

### 7-(2-isopropyl-2,7-diazaspiro[3.5]nonan-7-yl)-2-(2,2,2-trifluoroethyl)thiazolo[5,4-d]pyrimidine

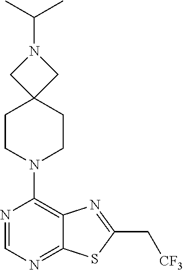 [SMILES: CC(C)N1CC2(CCN(c3ncnc4sc(CC(F)(F)F)nc34)CC2)C1]

### Step 1. N-(4,6-dichloropyrimidin-5-yl)-3,3,3-trifluoropropanamide

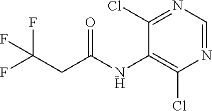 [SMILES: O=C(CC(F)(F)F)Nc1c(Cl)ncnc1Cl]

To a solution of 4,6-dichloropyrimidin-5-amine (1 g, 6.1 mmol) in CH₂ClCH₂Cl (50 mL) was added 3,3,3-trifluoropropanoyl chloride (1.7 g, 12.2 mmol) and PPh₃ (3.2 g, 12.2 mmol). The yellow solution was stirred at 60° C. for 4 h. The mixture was concentrated and purified by ISCO column (PE:EA=10:1 to 4:1) to afford N-(4,6-dichloropyrimidin-5-yl)-3,3,3-trifluoropropanamide (1.2 g). LCMS method E: Rₜ=0.70 min; (M+H)⁺=273.9.

### Step 2. tert-butyl 7-(6-chloro-5-(3,3,3-trifluoropropanamido)pyrimidin-4-yl)-2,7-diazaspiro[3.5]nonane-2-carboxylate

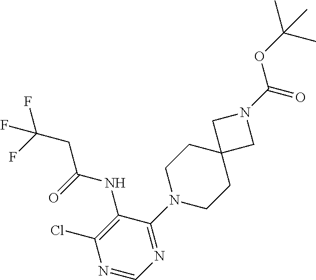 [SMILES: CC(C)(C)OC(=O)N1CC2(CCN(c3ncnc(Cl)c3NC(=O)CC(F)(F)F)CC2)C1]

To a solution of N-(4,6-dichloropyrimidin-5-yl)-3,3,3-trifluoropropanamide (500 mg, 1.82 mmol) in CH₃CN (20 mL) was added tert-butyl 2,7-diazaspiro[3.5]nonane-2-carboxylate (413 mg, 1.82 mmol) and Na₂CO₃ (386 mg, 3.64 mmol). The resulting mixture was stirred at 90° C. for about 3 h. The reaction mixture was filtered. The filtrate was concentrated in vacuo. The residue was purified by column chromatography on silica gel (petroleum ether:EtOAc=10:1 to 1:2) to give tert-butyl 7-(6-chloro-5-(3,3,3-trifluoropropanamido)pyrimidin-4-yl)-2,7-diazaspiro[3.5]nonane-2-carboxylate (540 mg). LCMS method C: Rₜ=0.78 min; (M+H)⁺=464.1; ¹H NMR (methanol-d4): δ 8.28 (s, 1H), 3.70 (s, 8H), 3.47 (q, J=10.4 Hz, 2H), 1.81-1.84 (m, 4H), 1.46 (s, 9H).

### Step 3. tert-butyl 7-(2-(2,2,2-trifluoroethyl)thiazolo[5,4-d]pyrimidin-7-yl)-2,7-diazaspiro[3.5]nonane-2-carboxylate

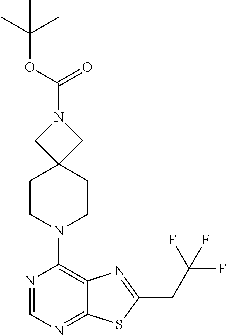 [SMILES: CC(C)(C)OC(=O)N1CC2(CCN(c3ncnc4sc(CC(F)(F)F)nc34)CC2)C1]

To a solution of tert-butyl 7-(6-chloro-5-(3,3,3-trifluoropropanamido)pyrimidin-4-yl)-2,7-diazaspiro[3.5]nonane-2-carboxylate (450 mg, 0.97 mmol) in toluene (15 mL, anhydrous) was added Lawesson's reagent (392 mg, 0.97 mmol). The resulting mixture was stirred at 100° C. under N₂ for about 4 h. The reaction mixture was concentrated in vacuo. The residue was purified by column chromatography on silica gel (petroleum ether:EtOAc=10:1 to 1:1) to give tert-butyl 7-(2-(2,2,2-trifluoroethyl)thiazolo[5,4-d]pyrimidin-7-yl)-2,7-diazaspiro[3.5]nonane-2-carboxylate (155 mg). LCMS method C: Rₜ=0.87 min; (M+H)⁺=444.1.

### Step 4. 7-(2,7-diazaspiro[3.5]nonan-7-yl)-2-(2,2,2-trifluoroethyl)thiazolo[5,4-d]pyrimidine

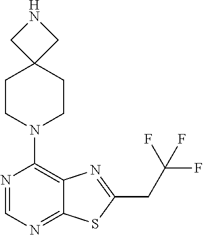 [SMILES: FC(F)(F)Cc1nc2c(N3CCC4(CC3)CNC4)ncnc2s1]

To a solution of tert-butyl 7-(2-(2,2,2-trifluoroethyl)thiazolo[5,4-d]pyrimidin-7-yl)-2,7-diazaspiro[3.5]nonane-2-carboxylate (50 mg, 0.11 mmol) in CH₂Cl₂ (4 mL, anhydrous) was added HCl-dioxane (4 N, 1 mL). The resulting mixture was stirred at 24-36° C. for about 4 h. The reaction mixture was concentrated in vacuo. The residue was purified by preparative RP-HPLC method D and dried by lyophilization to give 7-(2,7-diazaspiro[3.5]nonan-7-yl)-2-(2,2,2-trifluoroethyl)thiazolo[5,4-d]pyrimidine (30 mg). LCMS method C: Rₜ=0.61 min; (M+H)⁺=344.1.

### Step 5. (7-(2-isobutyl-2,7-diazaspiro[3.5]nonan-7-yl)-2-(2,2,2-trifluoroethyl) thiazolo[5,4-d]pyrimidine

To a solution of 7-(2,7-diazaspiro[3.5]nonan-7-yl)-2-(2,2,2-trifluoroethyl)thiazolo[5,4-d]pyrimidine (20 mg, 0.06 mmol) in anhydrous MeOH (2 mL) was added acetone (11 mg, 0.18 mmol) and the mixture was stirred at 17-24° C. for 30 min. Then NaBH₃CN (12 mg, 0.18 mmol) was added and the mixture was stirred at 17-24° C. for 3 h. The mixture was filtered and the filtrate was concentrated and purified by preparative RP-HPLC method B to give the title compound (12 mg). LCMS method C: Rₜ=0.64 min; (M+H)⁺=386.1; ¹H NMR (methanol-d4): 8.39 (s, 1H), 4.33-4.43 (m, 4H), 4.06-4.20 (m, 4H), 3.94-4.04 (m, 2H), 3.45-3.52 (m, 1H), 1.93-2.06 (m, 4H), 1.27 (d, J=6.4 Hz, 6H); ¹⁹F NMR (methanol-d4): −66.12 (s), −77.17 (s).

### Example 81

### 7-(2-isobutyl-2,7-diazaspiro[3.5]nonan-7-yl)-2-(2,2,2-trifluoroethyl) thiazolo[5,4-d]pyrimidine

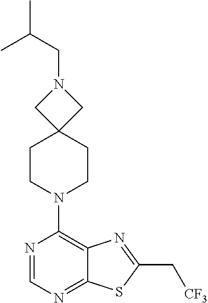 [SMILES: CC(C)CN1CC2(CCN(c3ncnc4sc(CC(F)(F)F)nc34)CC2)C1]

The title compound was prepared using procedures analogous to those described in Example 80, Step 5 by reductive amination between 7-(2,7-diazaspiro[3.5]nonan-7-yl)-2-(2,2,2-trifluoroethyl)thiazolo[5,4-d]pyrimidine (200 mg, 0.83 mmol) and isobutyraldehyde. LCMS method E: Rₜ=0.87 min; (M+H)⁺=400.2; ¹H NMR (methanol-d4): δ 8.40 (s, 1H), 4.27-4.56 (m, 4H), 4.20-4.25 (m, 2H), 4.16 (q, J=10.4 Hz, 2H), 3.90-4.10 (m, 2H), 3.15-3.18 (m, 2H), 1.96-2.10 (m, 5H), 1.05 (d, J=6.8 Hz, 6H); ¹⁹F NMR (methanol-d4): δ −66.11, −77.01.

### Example 82

### 7-(7-benzyl-2,7-diazaspiro[4.4]nonan-2-yl)-2-(2,2,2-trifluoroethyl)thiazolo[5,4-d]pyrimidine

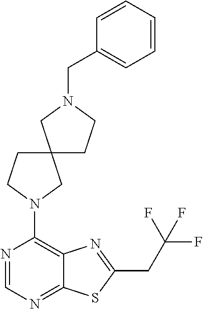 [SMILES: FC(F)(F)Cc1nc2c(N3CCC4(CCN(Cc5ccccc5)C4)C3)ncnc2s1]

### Step 1. tert-butyl 7-(6-chloro-5-(3,3,3-trifluoropropanamido)pyrimidin-4-yl)-2,7-diazaspiro[4.4]nonane-2-carboxylate

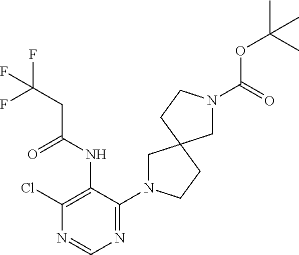 [SMILES: CC(C)(C)OC(=O)N1CCC2(CCN(c3ncnc(Cl)c3NC(=O)CC(F)(F)F)C2)C1]

To a solution of N-(4,6-dichloropyrimidin-5-yl)-3,3,3-trifluoropropanamide (see Example 80, Step 1; 500 mg, 1.82 mmol) in CH₃CN (20 mL) was added tert-butyl 2,7-diazaspiro[4.4]nonane-2-carboxylate (413 mg, 1.82 mmol) and Na₂CO₃ (386 mg, 3.64 mmol). The resulting mixture was stirred at 90° C. (oil temperature) for about 3 h. The reaction mixture was filtered and filtrate was concentrated in vacuo. The residue was purified by column chromatography on silica gel (petroleum ether:EtOAc=5:1 to 1:1) to give tert-butyl 7-(6-chloro-5-(3,3,3-trifluoropropanamido)pyrimidin-4-yl)-2,7-diazaspiro[4.4]nonane-2-carboxylate (550 mg). LCMS method C: Rₜ=0.77 min; (M+H)⁺=464.1.

### Step 2. tert-butyl 7-(2-(2,2,2-trifluoroethyl)thiazolo[5,4-d]pyrimidin-7-yl)-2,7-diazaspiro[4.4]nonane-2-carboxylate

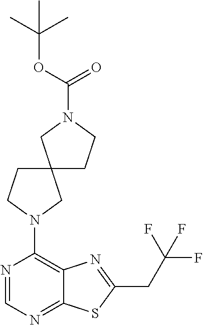 [SMILES: CC(C)(C)OC(=O)N1CCC2(CCN(c3ncnc4sc(CC(F)(F)F)nc34)C2)C1]

To a solution of tert-butyl 7-(6-chloro-5-(3,3,3-trifluoropropanamido)pyrimidin-4-yl)-2,7-diazaspiro[4.4]nonane-2-carboxylate (550 mg, 1.19 mmol) in toluene (20 mL, anhydrous) was added Lawesson reagent (480 mg, 1.19 mmol). The resulting mixture was stirred at 100° C. under N₂ for about 4 h. The reaction mixture was concentrated in vacuo. The residue was purified by column chromatography on silica gel (petroleum ether:EtOAc=10:1 to 5:1) to give tert-butyl 7-(2-(2,2,2-trifluoroethyl) thiazolo[5,4-d]pyrimidin-7-yl)-2,7-diazaspiro[4.4]nonane-2-carboxylate (350 mg). LCMS method C: Rₜ=0.85 min; (M+H)⁺=444.1.

### Step 3. 7-(2,7-diazaspiro[4.4]nonan-2-yl)-2-(2,2,2-trifluoroethyl)thiazolo[5,4-d]pyrimidine

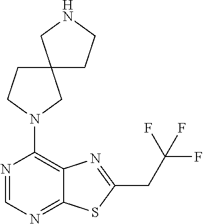 [SMILES: FC(F)(F)Cc1nc2c(N3CCC4(CCNC4)C3)ncnc2s1]

To a solution of tert-butyl 7-(2-(2,2,2-trifluoroethyl)thiazolo[5,4-d]pyrimidin-7-yl)-2,7-diazaspiro[4.4]nonane-2-carboxylate (350 mg, 0.79 mmol) in CH₂Cl₂ (10 mL, anhydrous) was added TFA (4 mL). The resulting mixture was stirred at RT for about 2 h. The reaction mixture was concentrated in vacuo to give 7-(2,7-diazaspiro[4.4]nonan-2-yl)-2-(2,2,2-trifluoroethyl)thiazolo[5,4-d]pyrimidine (460 mg). LCMS method C: Rₜ=0.56 min; (M+Na)⁺=343.9.

### Step 4. 7-(7-benzyl-2,7-diazaspiro[4.4]nonan-2-yl)-2-(2,2,2-trifluoroethyl)thiazolo[5,4-d]pyrimidine

To a solution of 7-(2,7-diazaspiro[4.4]nonan-2-yl)-2-(2,2,2-trifluoroethyl)thiazolo[5,4-d]pyrimidine (100 mg, 0.23 mmol) in MeOH (3 mL, anhydrous) was added benzaldehyde (29 mg, 0.27 mmol) and HOAc (0.1 mL). After stirring at 22-29° C. for 10 min, NaCNBH₃ (29 mg, 0.46 mmol) was added in one portion. The resulting mixture was stirred at 60° C. for 2 h. The reaction mixture was concentrated in vacuo. The residue was purified by preparative RP-HPLC method A and dried by lyophilization to give the title compound (32.0 mg) as a TFA salt. LCMS method E: Rₜ=0.87 min; (M+H)⁺=434.1; ¹H NMR (MeOH-d₄): δ 8.41 (s, 1H), 7.47-7.61 (m, 5H), 4.48 (s, 2H), 4.31 (s, 2H), 4.16 (q, J=10.4 Hz, 2H), 3.89 (s, 2H), 3.35-3.75 (m, 4H), 2.06-2.42 (m, 4H). ¹⁹F NMR (MeOH-d₄): δ −66.10, −77.21.

### Example 83

### 4-methyl-5-((7-(2-(2,2,2-trifluoroethyl)thiazolo[5,4-d]pyrimidin-7-yl)-2,7-diazaspiro[4.4]nonan-2-yl)methyl)-1H-indole-2-carbonitrile

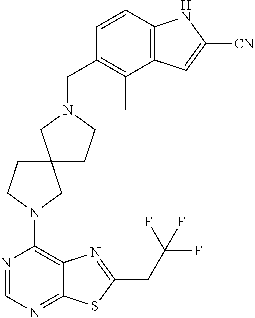 [SMILES: Cc1c(CN2CCC3(CCN(c4ncnc5sc(CC(F)(F)F)nc45)C3)C2)ccc2[nH]c(C#N)cc12]

The title compound was prepared using procedures analogous to those described in Example 82. In Step 4, 5-formyl-4-methyl-1H-indole-2-carbonitrile was used instead of benzaldehyde. LCMS method D: Rₜ=1.97 min; (M+H)⁺=512.2; ¹H NMR (MeOH-d₄, 400 MHz): δ 8.27-8.35 (m, 1H), 7.18-7.38 (m, 3H), 4.02-4.32 (m, 4H), 3.73-3.88 (m, 4H), 2.65-2.87 (m, 4H), 2.59 (s, 3H), 1.90-2.14 (m, 4H); ¹⁹F NMR (MeOH-d₄, 400 MHz): δ −66.15˜−66.09, −76.81˜−76.73.

### Example 84

### N-(4-((7-(2-(2,2,2-trifluoroethyl)thiazolo[5,4-d]pyrimidin-7-yl)-2,7-diazaspiro[4.4]nonan-2-yl)methyl)phenyl)methanesulfonamide

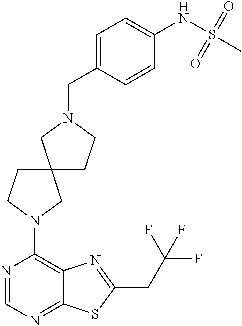 [SMILES: CS(=O)(=O)Nc1ccc(CN2CCC3(CCN(c4ncnc5sc(CC(F)(F)F)nc45)C3)C2)cc1]

### Step 1. 7-(7-(4-nitrobenzyl)-2,7-diazaspiro[4.4]nonan-2-yl)-2-(2,2,2-trifluoroethyl)thiazolo[5,4-d]pyrimidine

To a solution of 7-(2,7-diazaspiro[4.4]nonan-2-yl)-2-(2,2,2-trifluoroethyl)thiazolo[5,4-d]pyrimidine (see Example 28, Step 2) (50 mg, 0.15 mmol) in MeOH (2 mL, anhydrous) was added 4-nitrobenzaldehyde (27 mg, 0.17 mmol) and HOAc (9 mg, 0.15 mmol). After stirring at 23-28° C. for 30 min, NaCNBH₃ (19 mg, 0.30 mmol) was added in one portion. The resulting mixture was stirred at 50° C. (oil temperature) under N₂ for 20 h. LCMS determined the starting material was consumed completely. The reaction mixture was concentrated in vacuo. The residue was purified by basic preparative TLC (petroleum ether:EtOAc=1:1) to give 7-(7-(4-nitrobenzyl)-2,7-diazaspiro[4.4]nonan-2-yl)-2-(2,2,2-trifluoroethyl)thiazolo[5,4-d]pyrimidine as a yellow oil (50 mg, 70%). LCMS method C: Rₜ=0.68 min; (M+H)⁺=479.1.

### Step 2. 4-((7-(2-(2,2,2-trifluoroethyl)thiazolo[5,4-d]pyrimidin-7-yl)-2,7-diazaspiro[4.4]nonan-2-yl)methyl)aniline

To a solution of 7-(7-(4-nitrobenzyl)-2,7-diazaspiro[4.4]nonan-2-yl)-2-(2,2,2-trifluoroethyl) thiazolo[5,4-d]pyrimidine (50 mg, 0.10 mmol) in EtOAc (5 mL) was added Pd/C (10 mg, dry, 10%). The resulting mixture was stirred at 23-26° C. under H₂ (15 psi) for 20 h. LCMS determined the starting material was consumed completely. The reaction mixture was filtered. The filtrate was concentrated in vacuo to give 4-((7-(2-(2,2,2-trifluoroethyl)thiazolo[5,4-d]pyrimidin-7-yl)-2,7-diazaspiro[4.4]nonan-2-yl)methyl)aniline as a yellow oil (40 mg, 89%). LCMS method C: Rₜ=0.61 min; (M+H)⁺=449.1.

### Step 3. N-(4-((7-(2-(2,2,2-trifluoroethyl)thiazolo[5,4-d]pyrimidin-7-yl)-2,7-diazaspiro[4.4]nonan-2-yl)methyl)phenyl)methanesulfonamide

To a solution of 4-((7-(2-(2,2,2-trifluoroethyl)thiazolo[5,4-d]pyrimidin-7-yl)-2,7-diazaspiro[4.4] nonan-2-yl)methyl)aniline (40 mg, 0.089 mmol) in CH₂Cl₂ (2 mL, anhydrous) was added MsCl (11 mg, 0.089 mmol) and Et₃N (18 mg, 0.178 mmol). The resulting mixture was stirred at 24-28° C. under N₂ for 20 h. LCMS determined that 40% of starting the material remained. MsCl (11 mg, 0.089 mmol) was added. The resulting mixture was stirred at 24-28° C. under N₂ for 20 h. LCMS determined the starting material was consumed. The reaction mixture was concentrated in vacuo. The residue was purified by preparative RP-HPLC method A and dried by lyophilization to give the title compound (a TFA salt) as a colorless oil (3.6 mg, 6%). LCMS method C: Rₜ=0.66; (M+H)⁺=527.1; ¹H NMR (MeOH-d₄, 400 MHz): δ 8.40 (s, 1H), 7.53 (d, J=8.4 Hz, 2H), 7.36 (d, J=8.8 Hz, 2H), 4.23-4.47 (m, 4H), 4.10-4.19 (m, 2H), 3.37-4.08 (m, 6H), 3.03 (s, 3H), 2.12-2.34 (m, 4H); ¹⁹F NMR (MeOD, 400 MHz): δ −66.11, −77.12.

### Example 85

### 4-(2-isobutyl-2,7-diazaspiro[3.5]nonan-7-yl)-6-(2,2,2-trifluoroethyl)quinoline

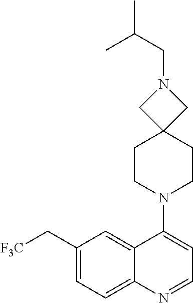 [SMILES: CC(C)CN1CC2(CCN(c3ccnc4ccc(CC(F)(F)F)cc34)CC2)C1]

### Step 1. 4-chloro-6-(4,4,5,5-tetramethyl-1,3,2-dioxaborolan-2-yl)quinoline

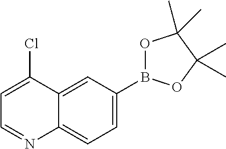 [SMILES: CC1(C)OB(c2ccc3nccc(Cl)c3c2)OC1(C)C]

To a solution of 6-bromo-4-chloroquinoline (CAS registry number 65340-70-7; 2 g, 8.4 mmol) in anhydrous dioxane (80 mL) was added 4,4,4′,4′,5,5,5′,5′-octamethyl-2,2′-bi(1,3,2-dioxaborolane) (2.36 g, 9.28 mmol), KOAc (1.6 g, 16.8 mmol) and Pd(dppf)Cl₂ (307 mg, 0.42 mmol) under N₂. The mixture was stirred at 90-100° C. for 16 h. The mixture was concentrated and the residue was purified by column chromatography on silica gel (petroleum ether:ethyl acetate=5:1) to give 4-chloro-6-(4,4,5,5-tetramethyl-1,3,2-dioxaborolan-2-yl)quinoline (2.1 g). LCMS method C: Rₜ=0.67 min; (M+H)⁺=436.0.

### Step 2. 4-chloro-6-(2,2,2-trifluoroethyl)quinoline

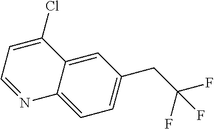 [SMILES: FC(F)(F)Cc1ccc2nccc(Cl)c2c1]

To 4-chloro-6-(4,4,5,5-tetramethyl-1,3,2-dioxaborolan-2-yl)quinoline (2.3 g, 7.95 mmol) in anhydrous dioxane (80 mL) was added 1,1,1-trifluoro-2-iodoethane (8.35 g, 39.8 mmol), X-phos (0.75 mg, 1.58 mmol), Cs₂CO₃ (112 mg, 1.164 mmol) and Pd₂(dba)₃ (18 mg, 0.019 mmol) under N₂. The mixture was stirred at 80-85° C. for 16 h. H₂O (300 mL) was poured into the mixture and the mixture was extracted with ethyl acetate (100 mL×3). The combined organic layers were washed with brine (200 mL), dried over Na₂SO₄, filtered and concentrated under reduced pressure. The residue was purified by preparative RP-HPLC method B and dried by lyophilization to give 4-chloro-6-(2,2,2-trifluoroethyl)quinoline (1.4 g). LCMS method C: Rₜ=0.76 min; (M+H)⁺=246.0; ¹H NMR (methanol-d4): δ 8.79 (d, J=4.8 Hz, 1H), 8.29 (s, 1H), 8.09 (d, J=8.8 Hz, 1H), 7.85 (d, J=8.4 Hz, 1H), 7.71 (d, J=4.8 Hz, 1H), 3.81 (q, J=10.8 Hz, 2H).

### Step 3. tert-butyl 7-(6-(2,2,2-trifluoroethyl)quinolin-4-yl)-2,7-diazaspiro[3.5]nonane-2-carboxylate

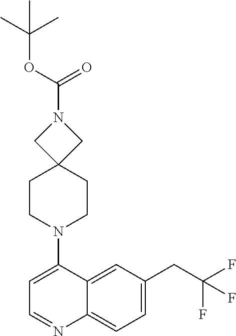 [SMILES: CC(C)(C)OC(=O)N1CC2(CCN(c3ccnc4ccc(CC(F)(F)F)cc34)CC2)C1]

A mixture of 4-chloro-6-(2,2,2-trifluoroethyl)quinoline (100 mg, 0.41 mmol), tert-butyl 2,7-diazaspiro[3.5]nonane-2-carboxylate (111 mg, 0.49 mmol), KI (136 mg, 0.82 mmol) and Et₃N (415 mg, 0.57 mL, 4.1 mmol) in anhydrous DMF (2 mL) was heated at 130° C. for 48 h. The mixture was added with H₂O (20 mL), extracted with ethyl acetate (3×30 mL). The combined organic layers were washed with H₂O (3×20 mL) and brine (20 mL), dried over anhydrous sodium sulfate, filtered and concentrated under reduced pressure. The residue was purified by column chromatograph on silica gel (eluting with petroleum ether:ethyl acetate=1:2 to 1:5) to give tert-butyl 7-(6-(2,2,2-trifluoroethyl)quinolin-4-yl)-2,7-diazaspiro[3.5]nonane-2-carboxylate (150 mg). LCMS method C: Rₜ=0.69 min; (M+H)⁺=436.0.

### Step 4. 4-(2,7-diazaspiro[3.5]nonan-7-yl)-6-(2,2,2-trifluoroethyl)quinoline

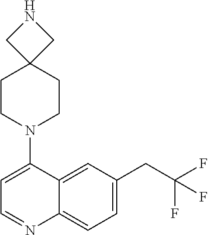 [SMILES: FC(F)(F)Cc1ccc2nccc(N3CCC4(CC3)CNC4)c2c1]

To a mixture of tert-butyl 7-(6-(2,2,2-trifluoroethyl)quinolin-4-yl)-2,7-diazaspiro[3.5]nonane-2-carboxylate (50 mg, 0.11 mmol) in anhydrous CH₂Cl₂ (3 mL) was added HCl/dioxane (4 N, 1 mL) under ice-cold water. The mixture was stirred at 21-25° C. for 1 h. The mixture was concentrated under reduced pressure and the residue was basified to pH=9-10 with 10% NaOH solution, extracted with ethyl acetate (3×30 mL). The combined organic layers were dried over anhydrous sodium sulfate, filtered and concentrated under reduced pressure to give 4-(2,7-diazaspiro[3.5]nonan-7-yl)-6-(2,2,2-trifluoroethyl)quinoline (35 mg, 90% crude yield) as a colorless oil, which was used in the next step directly without further purification. LCMS method C: Rₜ=0.50; (M+H)⁺=335.9.

### Step 5. 4-(2-isobutyl-2,7-diazaspiro[3.5]nonan-7-yl)-6-(2,2,2-trifluoroethyl)quinoline

A mixture of 4-(2,7-diazaspiro[3.5]nonan-7-yl)-6-(2,2,2-trifluoroethyl)quinoline (35 mg, 0.1 mmol, crude), isobutyraldehyde (22 mg, 0.3 mmol), NaBH₃CN (31 mg, 0.5 mmol) and HOAc (1 drop) in MeOH (3 mL) was stirred at 60° C. for 3 h. LCMS showed the reaction was completed. The mixture was concentrated under reduced pressure and the residue was purified by preparative RP-HPLC method D and dried by lyophilization to give the title compound (21 mg, 51%). LCMS method C: Rₜ=0.59 min; (M+H)⁺=392.2; ¹H NMR: (methanol-d4) δ 8.62 (d, J=5.2 Hz, 1H), 8.04 (s, 1H), 7.95 (d, J=8.8 Hz, 1H), 7.68 (d, J=8.8 Hz, 1H), 7.02 (d, J=5.6 Hz, 1H), 3.74 (q, J=11.2 Hz, 2H), 3.34 (s, 4H), 3.20-3.25 (m, 4H), 2.45 (d, J=7.2 Hz, 2H), 2.06-2.18 (m, 4H), 1.70-1.79 (m, 1H), 0.94 (d, J=6.8 Hz, 6H); ¹⁹F NMR (methanol-d4): δ −67.28.

### Example 86

### 4-methyl-5-((7-(6-(2,2,2-trifluoroethyl)quinolin-4-yl)-2,7-diazaspiro[4.4]nonan-2-yl)methyl)-1H-indole-2-carbonitrile (R and S Enantiomers)

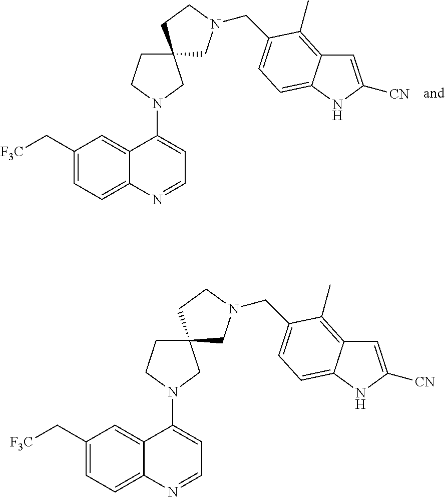 [SMILES: Cc1c(CN2CC[C@@]3(CCN(c4ccnc5ccc(CC(F)(F)F)cc45)C3)C2)ccc2[nH]c(C#N)cc12 ; Cc1c(CN2CC[C@]3(CCN(c4ccnc5ccc(CC(F)(F)F)cc45)C3)C2)ccc2[nH]c(C#N)cc12]

The title compounds were synthesized from 4-chloro-6-(2,2,2-trifluoroethyl)quinoline and tert-butyl 2,7-diazaspiro[4.4]nonane-2-carboxylate using procedures analogous to those described in Example 85 utilizing Step 3 through 5. In Step 5, 5-formyl-4-methyl-1H-indole-2-carbonitrile was used instead of isobutyraldehyde. The two isomers in the final compound were separated by preparative SFC separation (Method A) to give two separate isomers.

Isomer 1 (Example 86a): LCMS method C: Rₜ=0.60 min; (M+H)⁺=504.1; ¹H NMR (CD₃OD): δ 8.32 (d, J=5.6 Hz, 1H), 8.25 (s, 1H), 7.82 (d, J=8.8 Hz, 1H), 7.59 (d, J=8.8 Hz, 1H), 7.30 (d, J=8.8 Hz, 1H), 7.25 (s, 1H), 7.21 (d, J=7.6 Hz, 1H), 6.56 (d, J=5.6 Hz, 1H), 3.64-3.79 (m, 8H), 2.62-2.77 (m, 3H), 2.59-2.61 (m, 1H), 2.56 (s, 3H), 2.05-2.09 (m, 2H), 1.87-1.94 (m, 2H); ¹⁹F NMR (CD₃OD): δ −67.35; SFC: Rₜ=0.60 min, EE=92.09%.

Isomer 2 (Example 86b): LCMS method C: Rₜ=0.59 min; (M+H)⁺=504.1; ¹H NMR (CD₃OD): δ 8.28-8.33 (m, 2H), 7.83 (d, J=8.8 Hz, 1H), 7.64 (d, J=8.0 Hz, 1H), 7.20-7.39 (m, 3H), 6.59-6.61 (m, 1H), 3.67-3.84 (m, 9H), 2.60-2.77 (m, 3H), 2.57 (s, 3H), 2.07-2.12 (m, 2H), 1.90-1.96 (m, 2H); ¹⁹F NMR (CD₃OD): δ −67.35; SFC: Rₜ=6.56 min, EE=100%.

### Example 87

### 4-(2-(cyclopropylmethyl)-2,7-diazaspiro[3.5]nonan-7-yl)-6-(2,2,2-trifluoroethyl)quinoline

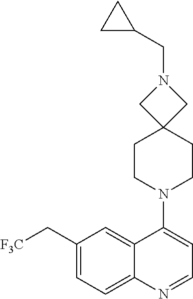 [SMILES: FC(F)(F)Cc1ccc2nccc(N3CCC4(CC3)CN(CC3CC3)C4)c2c1]

A mixture of 4-(2,7-diazaspiro[3.5]nonan-7-yl)-6-(2,2,2-trifluoroethyl)quinoline (35 mg, 0.1 mmol), (bromomethyl)cyclopropane (CAS registry number 7051-34-5; 14 mg, 0.1 mmol), KI (33 mg, 0.2 mmol), and Et₃N (101 mg, 1 mmol) in anhydrous DMF (2 mL) was stirred at 120° C. (oil bath) for 18 h. LCMS showed about 30% product was formed. The mixture was added with H₂O (20 mL), extracted with ethyl acetate (2×30 mL). The combined organic layers were washed with H₂O (2×20 mL) and brine (20 mL), dried over anhydrous sodium sulfate, filtered and concentrated under reduced pressure. The residue was purified by preparative RP-HPLC method A and dried by lyophilization to give the title compound (10 mg). LCMS method C: Rₜ=0.54 min; (M+H)⁺=389.9; ¹H NMR: (methanol-d4) δ 8.57 (d, J=7.2 Hz, 1H), 8.15 (s, 1H), 7.99 (s, 2H), 7.27 (d, J=6.8 Hz, 1H), 4.25-4.30 (m, 2H), 4.06-4.11 (m, 2H), 3.79-3.91 (m, 6H), 3.20 (d, J=7.6 Hz, 2H), 2.18-2.32 (m, 4H), 1.06-1.11 (m, 1H), 0.69-0.76 (m, 2H), 0.43-0.49 (m, 2H); ¹⁹F NMR (methanol-d4): δ −67.25, −76.99.

### Example 88

### 5,5-dimethyl-2-(6-(6-(trifluoromethoxy)quinazolin-4-yl)-2,6-diazaspiro[3.3]heptan-2-yl)-4,5-dihydrothiazole

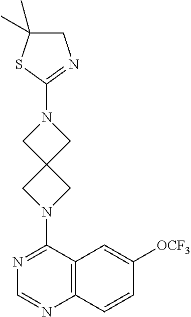 [SMILES: CC1(C)CN=C(N2CC3(C2)CN(c2ncnc4ccc(OC(F)(F)F)cc24)C3)S1]

### Step 1. tert-butyl 6-(6-(trifluoromethoxy)quinazolin-4-yl)-2,6-diazaspiro[3.3]heptane-2-carboxylate

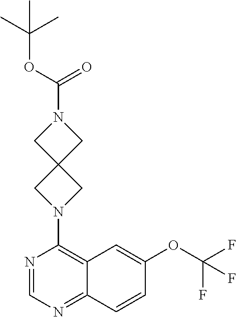 [SMILES: CC(C)(C)OC(=O)N1CC2(C1)CN(c1ncnc3ccc(OC(F)(F)F)cc13)C2]

A mixture of 4-chloro-6-(trifluoromethoxy)quinazoline (80 mg, 0.32 mmol), tert-butyl 2,6-diazaspiro[3.3]heptane-2-carboxylate (63 mg, 0.32 mmol) and Na₂CO₃ (102 mg, 0.96 mmol) in anhydrous DMF (5 mL) was stirred at 100° C. for 18 h. The mixture was added with H₂O (30 mL), extracted with ethyl acetate (3×40 mL). The combined organic layers were washed with H₂O (3×40 mL) and brine (40 mL), dried over anhydrous sodium sulfate, filtered and concentrated under reduced pressure. The residue was purified by column chromatograph on silica gel (eluting with ethyl acetate) to give tert-butyl 6-(6-(trifluoromethoxy)quinazolin-4-yl)-2,6-diazaspiro[3.3]heptane-2-carboxylate (120 mg). LCMS method C: Rₜ=0.67 min; (M+H)⁺=410.9.

### Step 2. 4-(2,6-diazaspiro[3.3]heptan-2-yl)-6-(trifluoromethoxy)quinazoline

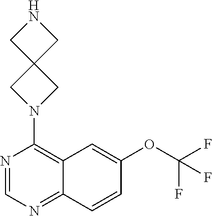 [SMILES: FC(F)(F)Oc1ccc2ncnc(N3CC4(CNC4)C3)c2c1]

To a mixture of tert-butyl 6-(6-(trifluoromethoxy)quinazolin-4-yl)-2,6-diazaspiro[3.3]heptane-2-carboxylate (120 mg, 0.29 mmol) in CH₂Cl₂ (3 mL) was added HCl-dioxane (4N, 1 mL) under ice-cold water. The mixture was stirred at 14-15° C. for 2 h. The mixture was concentrated under reduced pressure to give 4-(2,6-diazaspiro[3.3]heptan-2-yl)-6-(trifluoromethoxy)quinazoline as a white solid as HCl salt, which was used in the next step directly without further purification (101 mg). LCMS method D: Rₜ=1.85 min; (M+H)⁺=311.0.

### Step 3. N-(2-methylallyl)-6-(6-(trifluoromethoxy)quinazolin-4-yl)-2,6-diazaspiro[3.3]heptane-2-carbothioamide

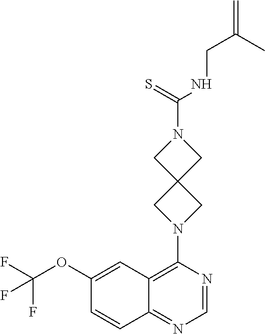 [SMILES: C=C(C)CNC(=S)N1CC2(C1)CN(c1ncnc3ccc(OC(F)(F)F)cc13)C2]

To a mixture of 4-(2,6-diazaspiro[3.3]heptan-2-yl)-6-(trifluoromethoxy)quinazoline (101 mg crude, 0.29 mmol) in anhydrous CH₂Cl₂ (6 mL) was added Et3N (147 mg, 0.2 mL, 1.45 mmol) and 3-isothiocyanato-2-methylprop-1-ene (50 mg, 0.44 mmol) under ice-cold water. The mixture was stirred at 14-15° C. for 2 h. The mixture was concentrated under reduced pressure. The residue was purified by column chromatograph on silica gel (eluting with ethyl acetate) to give N-(2-methylallyl)-6-(6-(trifluoromethoxy)quinazolin-4-yl)-2,6-diazaspiro[3.3]heptane-2-carbothioamide (60 mg). LCMS method C: Rₜ=0.64 min; (M+H)⁺=423.9; ¹H NMR (CDCl₃ 400 MHz): δ 8.59 (s, 1H), 7.84-7.86 (m, 1H), 7.53-7.55 (m, 2H), 5.10-5.11 (m, 1H), 4.80-4.84 (m, 2H), 4.65 (s, 4H), 4.32 (s, 4H), 4.14-4.16 (m, 2H), 1.94 (s, 3H).

### Step 4. 5,5-dimethyl-2-(6-(6-(trifluoromethoxy)quinazolin-4-yl)-2,6-diazaspiro[3.3]heptan-2-yl)-4,5-dihydrothiazole

A mixture of N-(2-methylallyl)-6-(6-(trifluoromethoxy)quinazolin-4-yl)-2,6-diazaspiro[3.3]heptane-2-carbothioamide (50 mg, 0.12 mmol) and concentrate HCl (1 mL) was stirred at 35° C. for 1 h. The mixture was basified to pH=8-9 with 10% NaOH solution and extracted with ethyl acetate (3×50 mL). The combined organic layers were washed with H₂O (30 mL) and brine (30 mL), dried over anhydrous sodium sulfate, filtered and concentrated under reduced pressure. The residue was purified by preparative RP-HPLC method D to give the title compound (3.4 mg). LCMS method C: Rₜ=0.57 min; (M+H)⁺=423.9.; ¹H NMR (CD₃OD): δ 8.47 (d, J=4.8 Hz, 1H), 7.84-7.86 (m, 2H), 7.75-7.77 (m, 1H), 4.78 (s, 4H), 4.24 (s, 4H), 3.63 (s, 2H), 1.54 (s, 6H); ¹⁹F NMR (CD₃OD): δ −59.591.

### Example 89

### 4-(2-isobutyl-2,7-diazaspiro[3.5]nonan-7-yl)-6-(2,2,2-trifluoroethyl) quinazoline

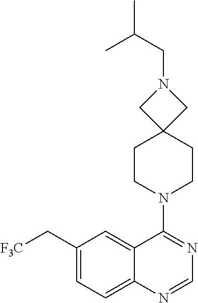 [SMILES: CC(C)CN1CC2(CCN(c3ncnc4ccc(CC(F)(F)F)cc34)CC2)C1]

### Step 1. methyl 2-((tert-butoxycarbonyl)amino)-5-(4,4,5,5-tetramethyl-1,3,2-dioxaborolan-2-yl)benzoate

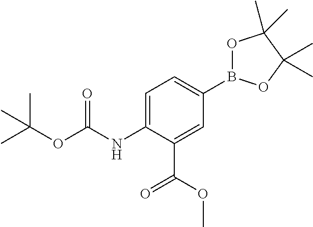 [SMILES: COC(=O)c1cc(B2OC(C)(C)C(C)(C)O2)ccc1NC(=O)OC(C)(C)C]

To a solution of methyl 5-bromo-2-((tert-butoxycarbonyl)amino)benzoate (1 g, 3.1 mmol) (See, ChemBioChem, 13(12), 1813-1817, S1813/1-S1813/75; 2012) in dioxane (20 mL) were added 4,4,4′,4′,5,5,5′,5′-octamethyl-2,2′-bi(1,3,2-dioxaborolane) (920 mg, 3.7 mmol), KOAc (600 mg, 6.2 mmol) and Pd(dppf)Cl₂ (225 mg, 0.31 mmol) and the mixture heated to 90° C. for 4 h under N₂. The mixture was cooled to 20-25° C. and filtered through silica gel to afford methyl 2-((tert-butoxycarbonyl)amino)-5-(4,4,5,5-tetramethyl-1,3,2-dioxaborolan-2-yl)benzoate (1.16 g), which was used in the next step without further purification. LCMS method C: Rₜ=0.97 min; (M+H)⁺=277.9.

### Step 2. methyl 2-((tert-butoxycarbonyl)amino)-5-(2,2,2-trifluoroethyl)benzoate

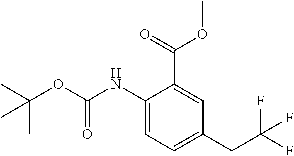 [SMILES: COC(=O)c1cc(CC(F)(F)F)ccc1NC(=O)OC(C)(C)C]

A mixture of methyl 2-((tert-butoxycarbonyl)amino)-5-(4,4,5,5-tetramethyl-1,3,2-dioxaborolan-2-yl)benzoate (1.15 g, 2.6 mmol), 1,1,1-trifluoro-2-iodoethane (2.18 g, 10.4 mmol), Pd₂(dba)₃ (950 mg, 1.04 mmol), Xantphos (1.0 g, 2.08 mmol) and Cs₂CO₃ (2.54 g, 7.8 mmol) in dioxane (100 mL) was heated at 90° C. for 16 h under N₂. The mixture was diluted with H₂O (50 mL) and extracted with EtOAc (50 mL×2). The combined organic layers were dried over Na₂SO₄, filtered and concentrated to give the crude product, which was purified by chromatography column (DCM:PE=1:10-1:5) to afford methyl 2-((tert-butoxycarbonyl)amino)-5-(2,2,2-trifluoroethyl)benzoate (820 mg). LCMS method CF: Rₜ=0.88 min; (M+H)⁺=233.7.

### Step 3. methyl 2-amino-5-(2,2,2-trifluoroethyl)benzoate

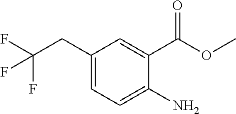 [SMILES: COC(=O)c1cc(CC(F)(F)F)ccc1N]

A mixture of methyl 2-((tert-butoxycarbonyl)amino)-5-(2,2,2-trifluoroethyl)benzoate (820 mg, 2.04 mmol) in HCl/MeOH (5 mL, 4 N) was stirred at 21-25° C. for 16 h. The mixture was concentrated, diluted with H₂O (50 mL), neutralized by Sat. NaHCO₃ (aq. 20 mL) and extracted with EtOAc (30 mL×2). The combined organic layers were dried over Na₂SO₄, filtered and concentrated to give methyl 2-amino-5-(2,2,2-trifluoroethyl)benzoate (450 mg). LCMS method C: Rₜ=0.72 min; (M+H)⁺=233.8.

### Step 4. 6-(2,2,2-trifluoroethyl)quinazolin-4-ol

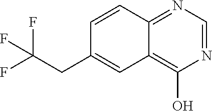 [SMILES: Oc1ncnc2ccc(CC(F)(F)F)cc12]

A mixture of methyl 2-amino-5-(2,2,2-trifluoroethyl)benzoate (450 mg, 1.93 mmol), ammonium formate (158 mg, 2.51 mmol) in formamide (10 mL) was heated at 140° C. for 16 h. The reaction was cooled to RT, diluted with H₂O (100 mL) and extracted with EtOAc (50 mL×2). The combined organic layers were dried over Na₂SO₄, filtered and concentrated to afford 6-(2,2,2-trifluoroethyl)quinazolin-4-ol (300 mg), which was used in the next step without purification. LCMS method C: Rₜ=0.62 min; (M+H)⁺=228.9.

### Step 5. 4-chloro-6-(2,2,2-trifluoroethyl)quinazohne

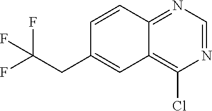 [SMILES: FC(F)(F)Cc1ccc2ncnc(Cl)c2c1]

A mixture of 6-(2,2,2-trifluoroethyl)quinazolin-4-ol (150 mg, 0.66 mmol) in SOCl₂ (3 mL) was heated at 80° C. for 1 h. The mixture was concentrated to afford 4-chloro-6-(2,2,2-trifluoroethyl)quinazoline (160 mg), which was used in the next step without purification. LCMS method C: Rₜ=0.72 min; (M+H)⁺=246.7.

### Step 6. tert-butyl 7-(6-(2,2,2-trifluoroethyl)quinazohn-4-yl)-2,7-diazaspiro[3.5]nonane-2-carboxylate

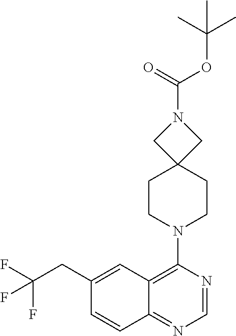 [SMILES: CC(C)(C)OC(=O)N1CC2(CCN(c3ncnc4ccc(CC(F)(F)F)cc34)CC2)C1]

A mixture of 4-chloro-6-(2,2,2-trifluoroethyl)quinazoline (80 mg, 0.3 mmol), tert-butyl 2,7-diazaspiro[3.5]nonane-2-carboxylate (66 mg, 0.3 mmol) and Na₂CO₃ (95 mg, 0.9 mmol) in CH₃CN (5 mL) was heated at 90° C. for 3 h. The mixture was diluted with H₂O (50 mL) and extracted with EtOAc (30 mL×2). The combined organic layer were dried over Na₂SO₄, filtered and concentrated to give the crude product, which was purified by preparative TLC (PE:EA=1:1) to afford tert-butyl 7-(6-(2,2,2-trifluoroethyl) quinazolin-4-yl)-2,7-diazaspiro[3.5]nonane-2-carboxylate (40 mg). LCMS method C: Rₜ=0.67 min; (M+H)⁺=437.1.

### Step 7. 4-(2,7-diazaspiro[3.5]nonan-7-yl)-6-(2,2,2-trifluoroethyl)quinazoline

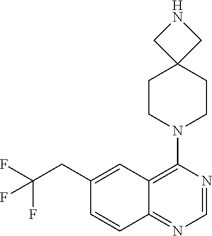 [SMILES: FC(F)(F)Cc1ccc2ncnc(N3CCC4(CC3)CNC4)c2c1]

To a solution of tert-butyl 7-(6-(2,2,2-trifluoroethyl)quinazolin-4-yl)-2,7-diazaspiro[3.5]nonane-2-carboxylate (40 mg, 0.092 mmol) in DCM (3 mL) was added TFA (0.5 mL). The reaction was stirred at 24-33° C. for 4 h. The mixture was concentrated, diluted with DCM:MeOH=10:1 (20 mL) and neutralized with saturated NaHCO₃ (aq. 20 mL). The separated organic layers were dried over Na₂SO₄, filtered and concentrated to afford 4-(2,7-diazaspiro[3.5]nonan-7-yl)-6-(2,2,2-trifluoroethyl)quinazoline (30 mg), which was used in the next step without purification. LCMS method C: Rₜ=0.30 min; (M+H)⁺=336.9.

### Step 8. 4-(2,7-diazaspiro[3.5]nonan-7-yl)-6-(2,2,2-trifluoroethyl)quinazoline

To a solution of 4-(2,7-diazaspiro[3.5]nonan-7-yl)-6-(2,2,2-trifluoroethyl)quinazoline (30 mg, 0.076 mmol) in MeOH (3 mL) were added isobutyraldehyde (6.5 mg, 0.091 mmol), NaBH₃CN (9.6 mg, 0.152 mmol) and HOAc (1 drop). Then the reaction was heated at 50° C. for 2 h under N₂. The mixture was concentrated and purified by preparative RP-LCMS method D to afford the title compound (13.5 mg). LCMS method D: Rₜ=1.77 min; (M+H)⁺=393.2; ¹H NMR (CD₃OD): δ 8.54 (s, 1H), 7.95 (s, 1H), 7.77 (s, 2H), 3.76-3.78 (m, 4H), 3.71-3.75 (m, 2H), 3.24 (s, 4H), 2.42-2.44 (m, 2H), 1.96-1.99 (m, 4H), 1.68-1.75 (m, 1H), 0.92 (d, J=6.4 Hz, 6H); ¹⁹F NMR (CD₃OD): δ −67.410.

### Example 90

### 4-methyl-5-((7-(6-(2,2,2-trifluoroethyl)quinazolin-4-yl)-2,7-diazaspiro[4.4]nonan-2-yl)methyl)-1H-indole-2-carbonitrile

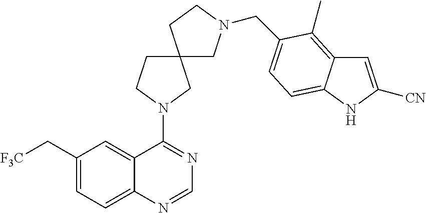 [SMILES: Cc1c(CN2CCC3(CCN(c4ncnc5ccc(CC(F)(F)F)cc45)C3)C2)ccc2[nH]c(C#N)cc12]

The title compound was synthesized from 4-chloro-6-(2,2,2-trifluoroethyl)quinazoline and tert-butyl 2,7-diazaspiro[4.4]nonane-2-carboxylate using procedures analogous to those described in Example 89, Steps 6-8. In Step 8, 5-formyl-4-methyl-1H-indole-2-carbonitrile was used instead of isobutyraldehyde. LCMS method D: Rₜ=1.77 min; (M+H)⁺=505.2; ¹H NMR (CD₃OD): δ 8.38 (s, 1H), 8.22 (s, 1H), 7.65-7.75 (m, 2H), 7.29 (d, J=7.6 Hz, 1H), 7.24 (s, 1H), 7.19 (d, J=8.4 Hz, 1H), 3.82-4.02 (m, 4H), 3.66-3.78 (m, 4H), 2.73-2.82 (m, 1H), 2.64-2.72 (m, 2H), 2.55-2.60 (m, 1H), 2.55 (s, 3H), 2.04-2.12 (m, 2H), 1.90 (t, J=6.8 Hz, 2H); ¹⁹F NMR (CD₃OD): δ −67.364.

### Example 91

### 4-(6-((tetrahydro-2H-pyran-4-yl)methyl)-2,6-diazaspiro[3.3]heptan-2-yl)-6-(2,2,2-trifluoroethyl)quinazoline

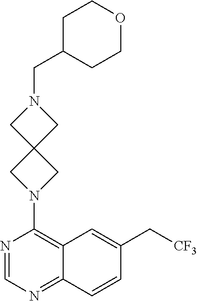 [SMILES: FC(F)(F)Cc1ccc2ncnc(N3CC4(CN(CC5CCOCC5)C4)C3)c2c1]

The title compound was synthesized from 4-chloro-6-(2,2,2-trifluoroethyl)quinazoline and tert-butyl 2,6-diazaspiro[3.3]heptane-2-carboxylate using procedures analogous to those described in Example 89, Steps 6-8. In Step 8, tetrahydro-2H-pyran-4-carbaldehyde was used instead of isobutyraldehyde. LCMS method D: Rₜ=1.61 min; (M+H)⁺=407.1; ¹H NMR (MeOD): δ 8.69 (s, 1H), 7.97-8.07 (m, 2H), 7.81 (d, J=8.8 Hz, 1H), 5.25 (s, 1H), 4.24-4.79 (m, 7H), 3.96 (q, J=11.2 Hz, 2H), 3.75-3.87 (m, 2H), 3.36-3.49 (m, 2H), 3.19 (d, J=6.8 Hz, 2H), 1.86-2.03 (m, 1H), 1.66 (d, J=10.8 Hz, 2H), 1.27-1.42 (m, 2H); ¹⁹F NMR (MeOD): δ −67.21, −76.95.

### Example 92

### 4-methyl-5-((2-(6-(2,2,2-trifluoroethyl)thieno[2,3-d]pyrimidin-4-yl)-2,6-diazaspiro[3.4]octan-6-yl)methyl)-1H-indole-2-carbonitrile

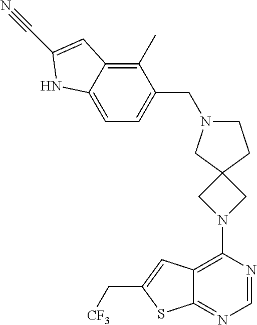 [SMILES: Cc1c(CN2CCC3(C2)CN(c2ncnc4sc(CC(F)(F)F)cc24)C3)ccc2[nH]c(C#N)cc12]

The title compound was synthesized from 4-chloro-6-(2,2,2-trifluoroethyl)thieno[2,3-d]pyrimidine and tert-butyl 2,6-diazaspiro[3.4]octane-6-carboxylate (CAS registry number 885270-84-8) using procedures analogous to those described in Example 1, Steps 4 through 6. In Step 6, 5-formyl-4-methyl-1H-indole-2-carbonitrile was used instead of benzaldehyde. LCMS method B: Rₜ=1.01 min; (M+H)⁺=497.5; ¹H NMR (methanol-d4): δ 8.38 (s, 1H), 7.45 (d, J=8.4 Hz, 1H), 7.38 (m, 3H), 4.61 (s, 2H), 4.38-4.64 (m, 4H), 3.94 (m, 1H), 3.91 (q, J=10.4 Hz, 2H), 3.65 (m, 2H), 3.48 (m, 1H), 2.69 (s, 3H), 2.64 (m, 1H), 2.49 (m, 1H). ¹⁹F NMR (methanol-d4): δ −67.704 9t, J=10.4 Hz).

### Example 93

### 4-(6-((tetrahydro-2H-pyran-4-yl)methyl)-2,6-diazaspiro[3.4]octan-2-yl)-6-(2,2,2-trifluoroethyl)thieno[2,3-d]pyrimidine

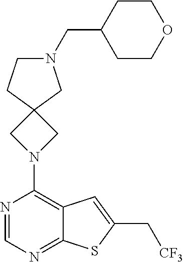 [SMILES: FC(F)(F)Cc1cc2c(N3CC4(CCN(CC5CCOCC5)C4)C3)ncnc2s1]

The title compound was synthesized from 4-chloro-6-(2,2,2-trifluoroethyl)thieno[2,3-d]pyrimidine and tert-butyl 2,6-diazaspiro[3.4]octane-6-carboxylate using procedures analogous to those described in Example 1,Steps 4 through 6. In Step 6, tetrahydro-2H-pyran-4-carbaldehyde was used instead of benzaldehyde. The title compound was isolated as a TFA salt. LCMS method B: Rₜ=0.80 min; (M+H)⁺=427.5; ¹H NMR (methanol-d4): δ 8.45 (s, 1H), 7.43 (s, 1H), 4.64 (m, 4H), 4.11 (m, 1H), 3.95 (m, 4H), 3.83 (m, 1H), 3.52 (m, 1H), 3.45 (m, 2H), 3.18 (d, J=6.8 Hz, 2H), 2.42-2.64 (m, 2H), 2.08 (m, 1H), 1.74 (m, 2H), 1.38 (m, 2H). ¹⁹F NMR (methanol-d4): δ −67.701 (t, J=10.4 Hz).

### Example 94

### 4-(6-(2-methylbutyl)-2,6-diazaspiro[3.4]octan-2-yl)-6-(2,2,2-trifluoroethyl)thieno[2,3-d]pyrimidine

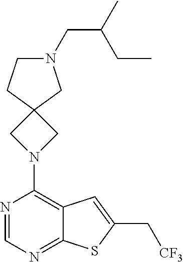 [SMILES: CCC(C)CN1CCC2(C1)CN(c1ncnc3sc(CC(F)(F)F)cc13)C2]

The title compound was synthesized from 4-chloro-6-(2,2,2-trifluoroethyl)thieno[2,3-d]pyrimidine and tert-butyl 2,6-diazaspiro[3.4]octane-6-carboxylate using procedures analogous to those described in Example 1, Steps 4 through 6. In step 6, 2-methylbutanal was used instead of benzaldehyde. The title compound was isolated as a TFA salt. LCMS method B: Rₜ=0.93 min; (M+H)⁺=399.5.

### Example 95

### 4-(6-benzyl-2,6-diazaspiro[3.4]octan-2-yl)-6-(2,2,2-trifluoroethyl)thieno[2,3-d]pyrimidine

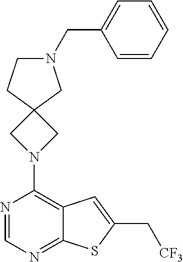 [SMILES: FC(F)(F)Cc1cc2c(N3CC4(CCN(Cc5ccccc5)C4)C3)ncnc2s1]

The title compound was synthesized from 4-chloro-6-(2,2,2-trifluoroethyl)thieno[2,3-d]pyrimidine and tert-butyl 2,6-diazaspiro[3.4]octane-6-carboxylate using procedures analogous to those described in Example 1, Steps 4 through 6. The title compound was isolated as a TFA salt. LCMS method B: Rₜ=0.95 min; (M+H)⁺=419.5.

### Example 96

### 4-methyl-5-((2-(6-(2,2,2-trifluoroethyl)thieno[2,3-d]pyrimidin-4-yl)-2,7-diazaspiro[3.5]nonan-7-yl)methyl)-1H-indole-2-carbonitrile hydrochloride

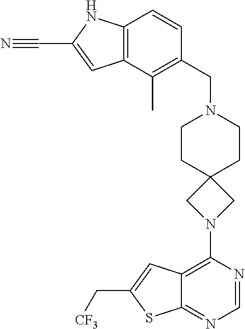 [SMILES: Cc1c(CN2CCC3(CC2)CN(c2ncnc4sc(CC(F)(F)F)cc24)C3)ccc2[nH]c(C#N)cc12]

The title compound was synthesized from 4-chloro-6-(2,2,2-trifluoroethyl)thieno[2,3-d]pyrimidine and tert-butyl 2,7-diazaspiro[3.5]nonane-7-carboxylate (CAS registry number 896464-16-7) using procedures analogous to those described in Example 1, Steps 4 through 6. In Step 6, 5-formyl-4-methyl-1H-indole-2-carbonitrile was used instead of benzaldehyde. The title compound was isolated as an HCl salt. LCMS method B: Rₜ=1.06 min; (M+H)⁺=511.6; ¹H NMR (methanol-d4): δ 8.52 (s, 1H), 7.55 (s, 1H), 7.48 (d, J=8.8 Hz, 1H), 7.42 (d, J=8.8 Hz, 1H), 7.41 (s, 1H), 4.10-4.70 (m, 4H), 4.53 (s, 2H), 3.98 (q, J=10.4 Hz, 2H), 3.56 (m, 2H), 3.26 (m, 2H), 2.70 (s, 3H), 2.35 (m, 2H), 2.12 (m, 2H). ¹⁹F NMR (methanol-d4): δ −67.659 (t, J=10.4 Hz).

### Example 97

### N-(4-((2-(6-(2,2,2-trifluoroethyl)thieno[2,3-d]pyrimidin-4-yl)-2,7-diazaspiro[3.5]nonan-7-yl)methyl)phenyl)methanesulfonamide

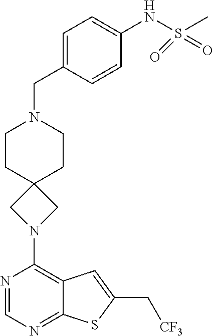 [SMILES: CS(=O)(=O)Nc1ccc(CN2CCC3(CC2)CN(c2ncnc4sc(CC(F)(F)F)cc24)C3)cc1]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. In step 6, N-(4-formylphenyl)methanesulfonamide was used instead of benzaldehyde. The title compound was isolated as a TFA salt. LCMS method B: Rₜ=1.01 min; (M+H)⁺=526.6.

### Example 98

### 4-(7-benzyl-2,7-diazaspiro[3.5]nonan-2-yl)-6-(2,2,2-trifluoroethyl)thieno[2,3-d]pyrimidine

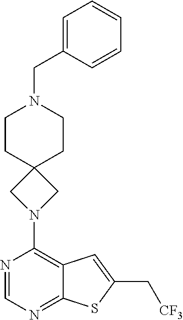 [SMILES: FC(F)(F)Cc1cc2c(N3CC4(CCN(Cc5ccccc5)CC4)C3)ncnc2s1]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. The title compound was isolated as a TFA salt. LCMS method B: Rₜ=0.95 min; (M+H)⁺=433.5.

### Example 99

### 4-(7-((tetrahydro-2H-pyran-4-yl)methyl)-2,7-diazaspiro[3.5]nonan-2-yl)-6-(2,2,2-trifluoroethyl)thieno[2,3-d]pyrimidine

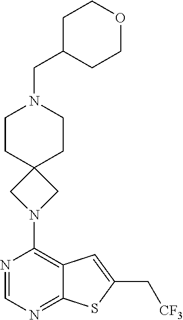 [SMILES: FC(F)(F)Cc1cc2c(N3CC4(CCN(CC5CCOCC5)CC4)C3)ncnc2s1]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. In Step 6, tetrahydro-2H-pyran-4-carbaldehyde was used instead of benzaldehyde. The title compound was isolated as a TFA salt. LCMS method B: Rₜ=0.83 min; (M+H)⁺=411.5.

### Example 100

### 4-(7-(2-methylbutyl)-2,7-diazaspiro[3.5]nonan-2-yl)-6-(2,2,2-trifluoroethyl)thieno[2,3-d]pyrimidine

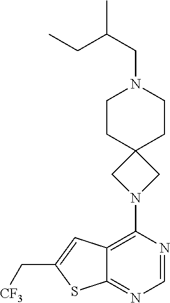 [SMILES: CCC(C)CN1CCC2(CC1)CN(c1ncnc3sc(CC(F)(F)F)cc13)C2]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. In Step 6, 2-methylbutanal was used instead of benzaldehyde. The title compound was isolated as a TFA salt. LCMS method B: Rₜ=0.94 min; (M+H)⁺=413.5.

### Example 101

### 4-(7-isobutyl-2,7-diazaspiro[3.5]nonan-2-yl)-6-(2,2,2-trifluoroethyl)thieno[2,3-d]pyrimidine

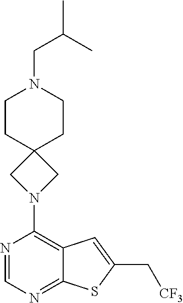 [SMILES: CC(C)CN1CCC2(CC1)CN(c1ncnc3sc(CC(F)(F)F)cc13)C2]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. In Step 6, isobutyraldehyde was used instead of benzaldehyde. The title compound was isolated as a TFA salt. LCMS method B: Rₜ=0.87 min; (M+H)⁺=399.5. ¹H NMR (methanol-d4): δ 8.42 (s, 1H), 7.46 (s, 1H), 4.38 (m, 4H), 3.94 (q, J=10.4 Hz, 2H), 3.60 (m, 2H), 3.06 (m, 2H), 3.00 (d, J=6. 8 Hz, 2H), 2.34 (m, 2H), 2.18 (m, 2H), 1.96 (m, 1H), 1.04 (m, 6H). ¹⁹F NMR (methanol-d4): δ −67.669 (t, J=10.4 Hz).

### Example 102

### 6-(2,2,2-trifluoroethyl)-4-(7-(((1r,4r)-4-(trifluoromethyl)cyclohexyl)methyl)-2,7-diazaspiro[3.5]nonan-2-yl)thieno[2,3-d]pyrimidine

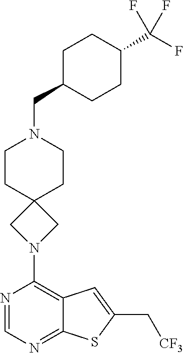 [SMILES: FC(F)(F)Cc1cc2c(N3CC4(CCN(C[C@H]5CC[C@H](C(F)(F)F)CC5)CC4)C3)ncnc2s1]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. In Step 6, (1r,4r)-4-(trifluoromethyl)cyclohexane-1-carbaldehyde was used instead of benzaldehyde. The title compound was isolated as a TFA salt. LCMS method B: Rₜ=1.15 min; (M+H)⁺=507.6.

### Example 103

### N-(4-((2-(6-(2,2,2-trifluoroethyl)thieno[2,3-d]pyrimidin-4-yl)-2,7-diazaspiro[3.5]nonan-7-yl)methyl)phenyl)acetamide

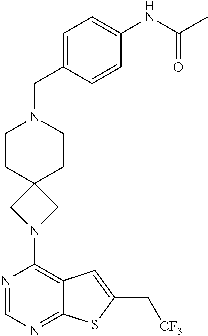 [SMILES: CC(=O)Nc1ccc(CN2CCC3(CC2)CN(c2ncnc4sc(CC(F)(F)F)cc24)C3)cc1]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Step 4 through 6. In Step 6, N-(4-formylphenyl)acetamide was used instead of benzaldehyde. The title compound was isolated as a TFA salt. LCMS method B: Rₜ=1.06 min; (M+H)⁺=490.7.

### Example 104

### 4-(2-(6-(2,2,2-trifluoroethyl)thieno[2,3-d]pyrimidin-4-yl)-2,7-diazaspiro[3.5]nonan-7-yl)tetrahydro-2H-thiopyran 1,1-dioxide

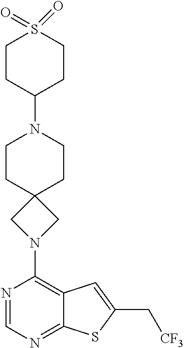 [SMILES: O=S1(=O)CCC(N2CCC3(CC2)CN(c2ncnc4sc(CC(F)(F)F)cc24)C3)CC1]

The title compound was synthesized from 4-chloro-6-(2,2,2-trifluoroethyl)thieno[2,3-d]pyrimidine and tert-butyl 2,7-diazaspiro[3.5]nonane-7-carboxylate using procedures analogous to those described in Example 1, Steps 4 through 6. In Step 6, tetrahydro-4H-thiopyran-4-one 1,1-dioxide (CAS registry number 17396-35-9) was used instead of benzaldehyde. The title compound was isolated as a TFA salt. LCMS method B: Rₜ=0.85 min; (M+H)⁺=475.6.

### Example 105

### N-((1r,4r)-4-((2-(6-(2,2,2-trifluoroethyl)thieno[2,3-d]pyrimidin-4-yl)-2,7-diazaspiro[3.5]nonan-7-yl)methyl)cyclohexyl)acetamide

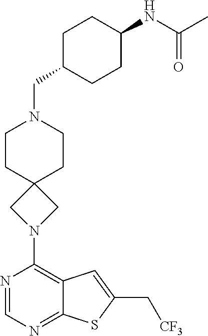 [SMILES: CC(=O)N[C@H]1CC[C@H](CN2CCC3(CC2)CN(c2ncnc4sc(CC(F)(F)F)cc24)C3)CC1]

The title compound was synthesized from 4-(2,7-diazaspiro[3.5]nonan-2-yl)-6-(2,2,2-trifluoroethyl)thieno[2,3-d]pyrimidine and tert-butyl ((1r,4r)-4-(hydroxymethyl)cyclohexyl)carbamate using procedures analogous to those described in Example 46, Steps 1 through 4. In Step 4, acetyl chloride was used instead of methyl carbonochloridate. The title compound was isolated as a TFA salt. LCMS method B: Rₜ=0.91 min; (M+H)⁺=496.7.

### Example 106

### N-((1r,4r)-4-((2-(6-(2,2,2-trifluoroethyl)thieno[2,3-d]pyrimidin-4-yl)-2,7-diazaspiro[3.5]nonan-7-yl)methyl)cyclohexyl)methanesulfonamide

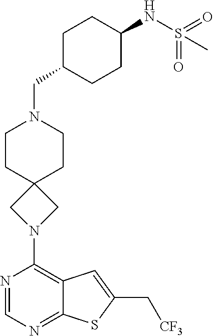 [SMILES: CS(=O)(=O)N[C@H]1CC[C@H](CN2CCC3(CC2)CN(c2ncnc4sc(CC(F)(F)F)cc24)C3)CC1]

The title compound was synthesized from 4-(2,7-diazaspiro[3.5]nonan-2-yl)-6-(2,2,2-trifluoroethyl)thieno[2,3-d]pyrimidine and tert-butyl ((1r,4r)-4-(hydroxymethyl)cyclohexyl)carbamate using procedures analogous to those described in Example 46, Steps 1 through 4. In Step 4, methanesulfonic anhydride was used instead of methyl carbonochloridate. The title compound was isolated as a TFA salt. LCMS method B: Rₜ=0.93 min; (M+H)⁺=532.6; ¹H NMR (DMSO-d6): δ 8.84 (br, 1H), 8.36 (s, 1H), 7.38 (s, 1H), 7.02 (d, 1H), 4.20 (m, 4H), 4.04 (q, J=10.4 Hz, 2H), 3.42 (m, 2H), 3.04 (m, 1H), 2.94 (m, 3H), 2.92 (s, 3H), 2.18 (m, 2H), 1.94 (m, 4H), 1.78 (m, 3H), 1.24 (m, 2H), 1.02 (m, 2H). ¹⁹F NMR (methanol-d4): δ −64.628 (t, J=10.4 Hz).

### Example 107

### methyl ((1r,4r)-4-((2-(6-(2,2,2-trifluoroethyl)thieno[2,3-d]pyrimidin-4-yl)-2,7-diazaspiro[3.5]nonan-7-yl)methyl)cyclohexyl)carbamate

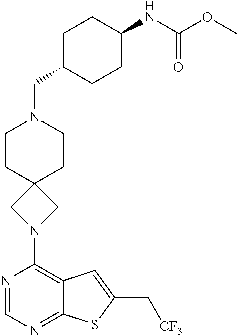 [SMILES: COC(=O)N[C@H]1CC[C@H](CN2CCC3(CC2)CN(c2ncnc4sc(CC(F)(F)F)cc24)C3)CC1]

The title compound was synthesized from 4-(2,7-diazaspiro[3.5]nonan-2-yl)-6-(2,2,2-trifluoroethyl)thieno[2,3-d]pyrimidine and tert-butyl ((1r,4r)-4-(hydroxymethyl)cyclohexyl)carbamate using procedures analogous to those described in Example 46, Steps 1 through 4. The title compound was isolated as a TFA salt. LCMS method B: Rₜ=0.96 min; (M+H)⁺=512.6.

### Example 108

### 4-(2-isobutyl-2,6-diazaspiro[3.4]octan-6-yl)-6-(2,2,2-trifluoroethyl)thieno[2,3-d]pyrimidine

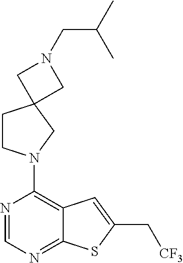 [SMILES: CC(C)CN1CC2(CCN(c3ncnc4sc(CC(F)(F)F)cc34)C2)C1]

The title compound was synthesized from 44-chloro-6-(2,2,2-trifluoroethyl)thieno[2,3-d]pyrimidine and tert-butyl 2,6-diazaspiro[3.4]octane-2-carboxylate (CAS registry number 885270-84-8) using procedures analogous to those described in Example 1, Steps 4 through 6. In Step 6, isobutyraldehyde was used instead of benzaldehyde. The title compound was isolated as a TFA salt. LCMS method B: Rₜ=0.88 min; (M+H)⁺=385.5; ¹H NMR (methanol-d4): δ 8.42 (s, 1H), 7.68 (s, 1H), 4.06-4.42 (m, 6H), 4.00 (m, 2H), 3.94 (q, J=10.4 Hz, 2H), 3.16 (d, J=6. 8 Hz, 2H), 2.48 (m, 2H), 1.98 (m, 1H), 1.04 (d, J=6.4 Hz, 6H). ¹⁹F NMR (methanol-d4): δ −67.722 (t, J=10.4 Hz).

### Example 109

### 4-(2-((tetrahydro-2H-pyran-4-yl)methyl)-2,6-diazaspiro[3.4]octan-6-yl)-6-(2,2,2-trifluoroethyl)thieno[2,3-d]pyrimidine

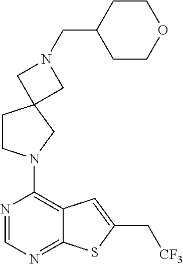 [SMILES: FC(F)(F)Cc1cc2c(N3CCC4(CN(CC5CCOCC5)C4)C3)ncnc2s1]

The title compound was synthesized from 4-chloro-6-(2,2,2-trifluoroethyl)thieno[2,3-d]pyrimidine and tert-butyl 2,6-diazaspiro[3.4]octane-2-carboxylate using procedures analogous to those described in Example 1, Steps 4 through 6. In Step 6, tetrahydro-2H-pyran-4-carbaldehyde was used instead of benzaldehyde. The title compound was isolated as a TFA salt. LCMS method B: Rₜ=0.81 min; (M+H)⁺=427.6.

### Example 110

### 4-(2-(2-methylbutyl)-2,6-diazaspiro[3.4]octan-6-yl)-6-(2,2,2-trifluoroethyl)thieno[2,3-d]pyrimidine

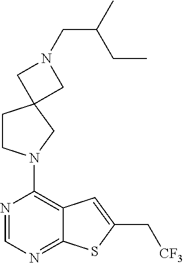 [SMILES: CCC(C)CN1CC2(CCN(c3ncnc4sc(CC(F)(F)F)cc34)C2)C1]

The title compound was synthesized from 4-chloro-6-(2,2,2-trifluoroethyl)thieno[2,3-d]pyrimidine and tert-butyl 2,6-diazaspiro[3.4]octane-2-carboxylate using procedures analogous to those described in Example 1, Steps 4 through 6. In Step 6, 2-methylbutanal was used instead of benzaldehyde. The title compound was isolated as a TFA salt. LCMS method B: Rₜ=0.94 min; (M+H)⁺=399.6.

### Example 111

### 4-(2-benzyl-2,6-diazaspiro[3.4]octan-6-yl)-6-(2,2,2-trifluoroethyl)thieno[2,3-d]pyrimidine

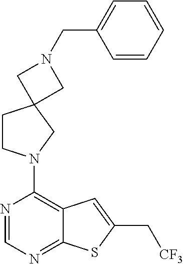 [SMILES: FC(F)(F)Cc1cc2c(N3CCC4(CN(Cc5ccccc5)C4)C3)ncnc2s1]

The title compound was synthesized from 4-chloro-6-(2,2,2-trifluoroethyl)thieno[2,3-d]pyrimidine and tert-butyl 2,6-diazaspiro[3.4]octane-2-carboxylate using procedures analogous to those described in Example 1, Steps 4 through 6. The title compound was isolated as a TFA salt. LCMS method B: Rₜ=0.94 min; (M+H)⁺=419.5.

### Example 112

### 4-methyl-5-((6-(6-(2,2,2-trifluoroethyl)thieno[2,3-d]pyrimidin-4-yl)-2,6-diazaspiro[3.4]octan-2-yl)methyl)-1H-indole-2-carbonitrile

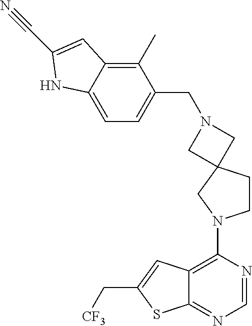 [SMILES: Cc1c(CN2CC3(CCN(c4ncnc5sc(CC(F)(F)F)cc45)C3)C2)ccc2[nH]c(C#N)cc12]

The title compound was synthesized from 4-chloro-6-(2,2,2-trifluoroethyl)thieno[2,3-d]pyrimidine and tert-butyl 2,6-diazaspiro[3.4]octane-2-carboxylate using procedures analogous to those described in Example 1, Steps 4 through 6. In Step 6, 5-formyl-4-methyl-1H-indole-2-carbonitrile was used instead of benzaldehyde. The title compound was isolated as a TFA salt. LCMS method B: Rₜ=1.03 min; (M+H)⁺=497.5.

### Example 113

### 4-(8-isobutyl-2,8-diazaspiro[4.5]decan-2-yl)-6-(2,2,2-trifluoroethyl)thieno[2,3-d]pyrimidine

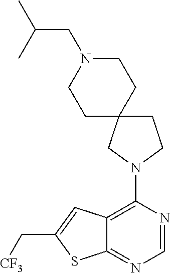 [SMILES: CC(C)CN1CCC2(CC1)CCN(c1ncnc3sc(CC(F)(F)F)cc13)C2]

The title compound was synthesized from 4-chloro-6-(2,2,2-trifluoroethyl)thieno[2,3-d]pyrimidine and tert-butyl 2,8-diazaspiro[4.5]decane-8-carboxylate (CAS registry number 236406-39-6) using procedures analogous to those described in Example 1, Step 4 through 6. In Step 6, isobutyraldehyde was used instead of benzaldehyde. The title compound was isolated as a TFA salt. LCMS method B: Rₜ=0.89 min; (M+H)⁺=413.5.

### Example 114

### 4-(8-(2-methylbutyl)-2,8-diazaspiro[4.5]decan-2-yl)-6-(2,2,2-trifluoroethyl)thieno[2,3-d]pyrimidine

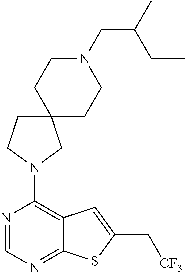 [SMILES: CCC(C)CN1CCC2(CC1)CCN(c1ncnc3sc(CC(F)(F)F)cc13)C2]

The title compound was synthesized from 4-chloro-6-(2,2,2-trifluoroethyl)thieno[2,3-d]pyrimidine and tert-butyl 2,8-diazaspiro[4.5]decane-8-carboxylate using procedures analogous to those described in Example 1, Steps 4 through 6. In Step 6, 2-methylbutanal was used instead of benzaldehyde. The title compound was isolated as a TFA salt. LCMS method B: Rₜ=0.98 min; (M+H)⁺=427.5.

### Example 115

### 4-(8-((tetrahydro-2H-pyran-4-yl)methyl)-2,8-diazaspiro[4.5]decan-2-yl)-6-(2,2,2-trifluoroethyl)thieno[2,3-d]pyrimidine

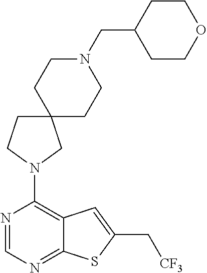 [SMILES: FC(F)(F)Cc1cc2c(N3CCC4(CCN(CC5CCOCC5)CC4)C3)ncnc2s1]

The title compound was synthesized from 4-chloro-6-(2,2,2-trifluoroethyl)thieno[2,3-d]pyrimidine and tert-butyl 2,8-diazaspiro[4.5]decane-8-carboxylate using procedures analogous to those described in Example 1, Steps 4 through 6. In Step 6, tetrahydro-2H-pyran-4-carbaldehyde was used instead of benzaldehyde. The title compound was isolated as a TFA salt. LCMS method B: Rₜ=0.87 min; (M+H)⁺=455.5.

### Example 116

### 4-(8-benzyl-2,8-diazaspiro[4.5]decan-2-yl)-6-(2,2,2-trifluoroethyl)thieno[2,3-d]pyrimidine

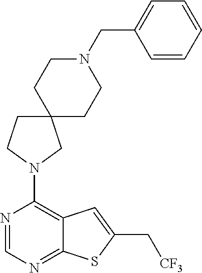 [SMILES: FC(F)(F)Cc1cc2c(N3CCC4(CCN(Cc5ccccc5)CC4)C3)ncnc2s1]

The title compound was synthesized from 4-chloro-6-(2,2,2-trifluoroethyl)thieno[2,3-d]pyrimidine and tert-butyl 2,8-diazaspiro[4.5]decane-8-carboxylate using procedures analogous to those described in Example 1, Steps 4 through 6. The title compound was isolated as a TFA salt. LCMS method B: Rₜ=0.97 min; (M+H)⁺=447.5.

### Example 117

### 4-methyl-5-((2-(6-(2,2,2-trifluoroethyl)thieno[2,3-d]pyrimidin-4-yl)-2,8-diazaspiro[4.5]decan-8-yl)methyl)-1H-indole-2-carbonitrile

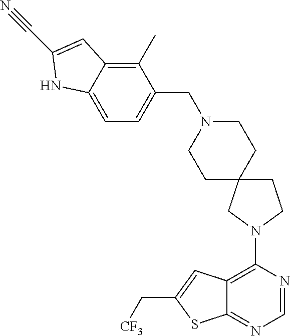 [SMILES: Cc1c(CN2CCC3(CC2)CCN(c2ncnc4sc(CC(F)(F)F)cc24)C3)ccc2[nH]c(C#N)cc12]

The title compound was synthesized from 4-chloro-6-(2,2,2-trifluoroethyl)thieno[2,3-d]pyrimidine and tert-butyl 2,8-diazaspiro[4.5]decane-8-carboxylate using procedures analogous to those described in Example 1, Steps 4 through 6. In Step 6, 5-formyl-4-methyl-1H-indole-2-carbonitrile was used instead of benzaldehyde. The title compound was isolated as a TFA salt. LCMS method B: Rt=1.05 min; (M+H)+=525.6.

### Example 118

### 7-((2-oxo-2,3-dihydro-1H-benzoldlimidazol-5-yl)methyl)-2-(6-(2,2,2-trifluoroethyl)thieno[2,3-d]pyrimidin-4-yl)-2,7-diazaspiro[4.4]nonan-3-one

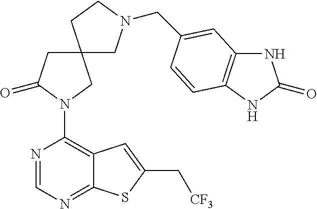 [SMILES: O=C1CC2(CCN(Cc3ccc4[nH]c(=O)[nH]c4c3)C2)CN1c1ncnc2sc(CC(F)(F)F)cc12]

### Step 1. tert-butyl 8-oxo-7-(6-(2,2,2-trifluoroethyl)thieno[2,3-dipyrimidin-4-yl)-2,7-diazaspiro[4.4]nonane-2-carboxylate

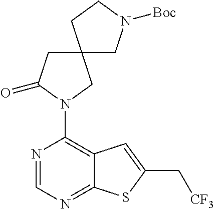 [SMILES: CC(C)(C)OC(=O)N1CCC2(CC(=O)N(c3ncnc4sc(CC(F)(F)F)cc34)C2)C1]

To a solution of 4-chloro-6-(2,2,2-trifluoroethyl)thieno[2,3-d]pyrimidine (130 mg, 0.51 mmol) in dioxane (4 mL) was added tert-butyl 8-oxo-2,7-diazaspiro[4.4]nonane-2-carboxylate (122 mg, 0.51 mmol) and Cs₂CO₃ (336 mg, 1.03 mmol). Then Pd₂dba₃ (24 mg, 0.026 mmol) and Xantphos (30 mg, 0.052 mmol) were added under N₂. The reaction was heated in a microwave at 100° C. for 1 h. LCMS showed about 65% of the desired compound was detected. The mixture was concentrated, diluted with H₂O (50 mL) and extracted with EtOAc (30 mL×2). The combined organic layers were dried over Na₂SO₄, filtered and concentrated to give the crude product, which was purified by preparative TLC on silica (PE:EA=2:1) to afford tert-butyl 8-oxo-7-(6-(2,2,2-trifluoroethyl)thieno[2,3-d]pyrimidin-4-yl)-2,7-diazaspiro[4.4]nonane-2-carboxylate (140 mg, 60%) as a yellow solid. LCMS Method C: t_{R}=0.85 min; [M-56+H]⁺=401.

### Step 2. 2-(6-(2,2,2-trifluoroethyl)thieno[2,3-d]pyrimidin-4-yl)-2,7-diazaspiro[4.4]nonan-3-one

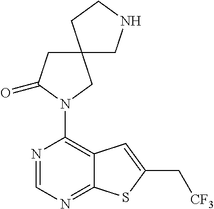 [SMILES: O=C1CC2(CCNC2)CN1c1ncnc2sc(CC(F)(F)F)cc12]

To a solution of tert-butyl 8-oxo-7-(6-(2,2,2-trifluoroethyl)thieno[2,3-d]pyrimidin-4-yl)-2,7-diaspiro[4.4]-nonane-2-carboxylate (60 mg, 0.13 mmol) in DCM (5 mL) was added HCl-dioxane (4 N in dioxane, 1 mL). The reaction was stirred at 20-26° C. for 2 h. LCMS showed about 80% of the desired compound. The mixture was neutralized with sat. aq. NaHCO₃ (30 mL) and extracted with DCM (30 mL×2). The combined organic layers were dried over Na₂SO₄, filtered and concentrated to give 2-(6-(2,2,2-trifluoroethyl)thieno[2,3-d]pyrimidin-4-yl)-2,7-diazaspiro[4.4]nonan-3-one (40 mg, 85%) as yellow oil, which was used for the next step without purification. LCMS method C: t_{R}=0.61 min; [M+H]⁺=357.

### Step 3. 7-((2-oxo-2,3-dihydro-1H-benzo[d]imidazol-5-yl)methyl)-2-(6-(2,2,2-trifluoroethyl)-thieno[2,3-d]pyrimidin-4-yl)-2,7-diazaspiro[4.4]nonan-3-one

To a solution of 2-(6-(2,2,2-trifluoroethyl)thieno[2,3-d]pyrimidin-4-yl)-2,7-diazaspiro[4.4]nonan-3-one (40 mg, 0.09 mmol, 80% purity) in MeOH (5 mL) wes added 2-oxo-2,3-dihydro-1H-benzo[d]imidazole-5-carbaldehyde (18 mg, 0.09 mmol) and NaBH₃CN (14 mg, 0.22 mmol). The resulting solution was stirred at 50° C. for 16 h. About 93% of the desired compound was detected by LCMS. The mixture was concentrated and purified by basic preparative RP-HPLC Method D to afford the title compound (7.9 mg, yield 18%) as a white solid. LCMS Method E: t_{R}=1.06 min; [M+H]⁺=503.1; ¹H NMR (MeOH_d₄): δ 8.83 (s, 1H), 7.53 (s, 1H), 7.14 (s, 1H), 7.09 (d, J=8.8 Hz, 1H), 7.02 (d, J=8.8 Hz, 1H), 4.61 (s, 3H), 4.08-4.14 (m, 2H), 3.97-3.40 (m, 2H), 3.87-3.94 (m, 2H), 2.97-3.04 (m, 1H), 2.31-2.39 (m, 3H), 2.04-2.07 (m, 1H); ¹⁹F NMR: (MeOH-d₄): δ −67.334.

### Example 119

### 1-(2-hydroxyethyl)-5-((7-(6-(2,2,2-trifluoroethyl)thieno[3,2-d]pyrimidin-4-yl)-2,7-diazaspiro[4.4]nonan-2-yl)methyl)-1H-benzo[d]imidazol-2(3H)-one

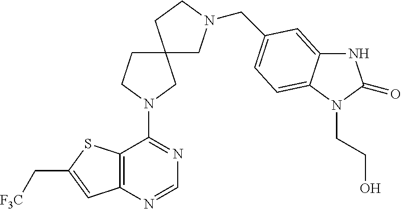 [SMILES: O=c1[nH]c2cc(CN3CCC4(CCN(c5ncnc6cc(CC(F)(F)F)sc56)C4)C3)ccc2n1CCO]

### Step 1. 1-(4-chlorothieno[3,2-d]pyrimidin-6-yl)-2,2,2-trifluoroethanone

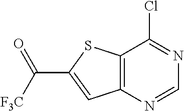 [SMILES: O=C(c1cc2ncnc(Cl)c2s1)C(F)(F)F]

To a solution of 6-bromo-4-chlorothieno[3,2-d]pyrimidine (4.0 g, 16.03 mmol) in THF (80 mL, anhydrous) was added n-butyllithium (n-BuLi, 7.1 mL, 17.63 mmol, 2.5 M in hexane) dropwise via syringe at −70° C. under N₂. After stirring at −70° C. for 30 min, 2,2,2-trifluoro-N-methoxy-N-methylacetamide (2.8 g, 17.63 mmol) was added in one portion via syringe. The resulting mixture was warmed to 18-23° C. and stirred for 2 h. The reaction mixture was diluted with 1 N HCl (15 mL) and extracted with EtOAc (3×20 mL). The combined organic layers were concentrated under reduced pressure and purified by flash column on silica gel (petroleum ether/ethyl acetate=1/1) to give crude 1-(4-chlorothieno[3,2-d]pyrimidin-6-yl)-2,2,2-trifluoroethanone as orange oil, which was used for the next step without further purification. Yield: 4.2 g (98% crude yield); LCMS method C: Rₜ=0.694 min; (M+18)⁺=284.8, 286.8 (chloro isotopes).

### Step 2. 1-(4-chlorothieno[3,2-d]pyrimidin-6-yl)-2,2,2-trifluoroethanol

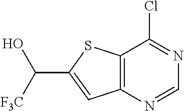 [SMILES: OC(c1cc2ncnc(Cl)c2s1)C(F)(F)F]

To a solution of 1-(4-chlorothieno[3,2-d]pyrimidin-6-yl)-2,2,2-trifluoroethanone (4.2 g, 15.75 mmol, crude) in MeOH (40 mL, anhydrous) was added NaBH₄ (359 mg, 9.45 mmol) in portions at 0-5° C. The resulting mixture was stirred at 18-21° C. for 40 min. The reaction mixture was diluted with brine (40 mL) and extracted with EtOAc (2×40 mL). The combined organic layers were dried over anhydrous Na₂SO₄, concentrated under reduced pressure, and purified by column chromatography on silica gel (petroleum ether/ethyl acetate=5/1 to 3/1) to give 1-(4-chlorothieno[3,2-d]pyrimidin-6-yl)-2,2,2-trifluoroethanol (Rf=0.2-0.3, petroleum ether/ethyl acetate=5/1) as yellow solid. Yield: 600 mg (14%); LCMS method C: Rₜ=0.727 min. (M+18)⁺=268.8, 270.8 (chloro isotopes); ¹H NMR (CD₃CN): δ ppm 8.97 (s, 1H), 7.68 (s, 1H), 5.65-5.70 (m, 1H). ¹⁹F NMR (CD₃CN): δ ppm −78.88.

### Step 3. O-(1-(4-chlorothieno[3,2-d]pyrimidin-6-yl)-2,2,2-trifluoroethyl)O-phenyl carbonothioate

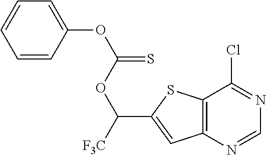 [SMILES: FC(F)(F)C(OC(=S)Oc1ccccc1)c1cc2ncnc(Cl)c2s1]

To a solution of 1-(4-chlorothieno[3,2-d]pyrimidin-6-yl)-2,2,2-trifluoroethanol (600 mg, 2.23 mmol) in CH₃CN (15 mL) was added Et₃N (271 mg, 2.68 mmol) and DMAP (27 mg, 0.22 mmol). The resulting mixture was cooled to 0-5° C. and then O-phenyl carbonochloridothioate (463 mg, 2.68 mmol) was added dropwise via syringe. The resulting mixture was stirred at 19-22° C. under N₂ for 1 h. The reaction mixture was concentrated and the residue was diluted with CH₂Cl₂ (15 mL) then filtered through silica gel. The filtrate was concentrated to give crude O-(1-(4-chlorothieno[3,2-d]pyrimidin-6-yl)-2,2,2-trifluoroethyl) O-phenyl carbonothioate as yellow oil. Yield: 460 mg (51%); LCMS method C: Rₜ=0.959 min. (M+H)⁺=404.8, 406.8 (chloro isotopes).

### Step 4. 4-chloro-6-(2,2,2-trifluoroethyl)thieno[3,2-d]pyrimidine

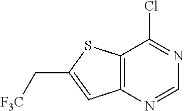 [SMILES: FC(F)(F)Cc1cc2ncnc(Cl)c2s1]

To a solution of O-(1-(4-chlorothieno[3,2-d]pyrimidin-6-yl)-2,2,2-trifluoroethyl) O-phenyl carbonothioate (460 mg, 1.14 mmol) in toluene (15 mL, anhydrous) was added Bu₃SnH (664 mg, 2.28 mmol) and azobisisobutyronitrile (AIBN, 94 mg, 0.57 mmol). The resulting mixture was degassed and refilled with N₂ for 3 times then stirred at 115° C. (oil temperature) under N₂ for 20 h. The mixture was concentrated and the residue was purified by column chromatography on silica gel (petroleum ether/ethyl acetate=20/1 to 3/1) to give 4-chloro-6-(2,2,2-trifluoroethyl)thieno[3,2-d]pyrimidine as yellow solid. Yield: 150 mg (52%); LCMS method C: Rₜ=0.675 min. (M+H)⁺=252.8); ¹H NMR (CDCl₃): δ ppm 8.98 (s, 1H), 7.53 (s, 1H), 3.79 (q, J=10.0 Hz, 2H). ¹⁹F NMR (CD₃CN): δ ppm −66.27.

### Step 5. tert-butyl 7-(6-(2,2,2-trifluoroethyl)thieno[3,2-d]pyrimidin-4-yl)-2,7-diazaspiro[4.4]nonane-2-carboxylate

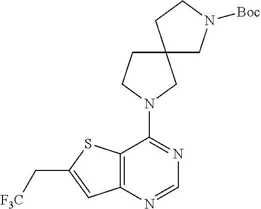 [SMILES: CC(C)(C)OC(=O)N1CCC2(CCN(c3ncnc4cc(CC(F)(F)F)sc34)C2)C1]

To a solution of 4-chloro-6-(2,2,2-trifluoroethyl)thieno[3,2-d]pyrimidine (100 mg, 0.4 mmol) and tert-butyl 2,7-diazaspiro[4.4]nonane-2-carboxylate, semi oxalate salt (119 mg, 0.44 mmol) in MeCN (5 mL) was added K₂CO₃ (218 mg, 1.6 mmol). The resulting mixture was stirred at 90° C. (oil bath) for 16 h. The mixture was filtered and the filtrate was concentrated then the residue was purified by flash chromatography on silica gel (petroleum ether/ethyl acetate=100/1 to 0/100) to give tert-butyl 7-(6-(2,2,2-trifluoroethyl)thieno[3,2-d]pyrimidin-4-yl)-2,7-diazaspiro[4.4] nonane-2-carboxylate as yellow oil. Yield: 50 mg (28%); LCMS method C: Rₜ=0.730 min. (M+H)⁺=443.0; ¹H NMR (CDCl₃): δ 8.51 (s, 1H), 7.34 (s, 1H), 3.95-4.20 (m, 2H), 3.65-3.90 (m, 4H), 3.25-3.60 (m, 4H), 1.90-2.20 (m, 4H), 1.47 (s, 9H).

### Step 6. 4-(2,7-diazaspiro[4.4]nonan-2-yl)-6-(2,2,2-trifluoroethyl)thieno[3,2-d]pyrimidine

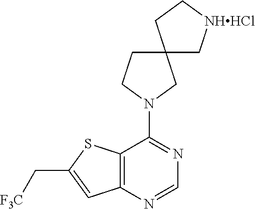

A solution of tert-butyl 7-(6-(2,2,2-trifluoroethyl)thieno[3,2-d]pyrimidin-4-yl)-2,7-diazaspiro[4.4]nonane-2-carboxylate (50 mg, 0.11 mmol) in HCl solution (4 M in dioxane, 2 mL) was stirred at 10-15° C. for about 2 h. The resulting mixture was concentrated to give crude 4-(2,7-diazaspiro[4.4]nonan-2-yl)-6-(2,2,2-trifluoroethyl)thieno[3,2-d]pyrimidine (HCl salt) as yellow solid, which was used for next step without further purification. Yield: 50 mg (100% crude, HCl salt); LCMS method C: Rₜ=0.313 min. (M+H)⁺=343.1);

### Step 7. 1-(2-hydroxyethyl)-5-((7-(6-(2,2,2-trifluoroethyl)thieno[3,2-d]pyrimidin-4-yl)-2,7-diazaspiro[4.4]nonan-2-yl)methyl)-1H-benzo[d]imidazol-2(3H)-one

A solution of 4-(2,7-diazaspiro[4.4]nonan-2-yl)-6-(2,2,2-trifluoroethyl)thieno[3,2-d]pyrimidine (50 mg, 0.11 mmol, HCl salt, crude) and triethylamine (13 mg, 0.12 mmol) in MeOH (2 mL, anhydrous) was stirred at 10-15° C. for 30 min. Then 1-(2-hydroxyethyl)-2-oxo-2,3-dihydro-1H-benzo[d]imidazole-5-carbaldehyde (24 mg, 0.11 mmol) and NaBH₃CN (29 mg, 0.47 mmol) was added. The resulting mixture was stirred at 10-15° C. for about 16 h. The resulting mixture was concentrated and purified by basic preparative RP-HPLC method D to give 1-(2-hydroxyethyl)-5-((7-(6-(2,2,2-trifluoroethypthieno[3,2-d]pyrimidin-4-yl)-2,7-diazaspiro[4.4]nonan-2-yl)methyl)-1H-benzo[d]imidazol-2(3H)-one as white solid. Yield: 20 mg (34%); LCMS method C: Rₜ=0.857 min. (M+H)⁺=533.3; ¹H NMR (CD₃OD): δ ppm 8.31 (s, 1H), 7.30 (s, 1H), 7.00-7.15 (m, 3H), 3.70-4.05 (m, 10H), 3.66 (s, 2H), 2.75-2.80 (m, 1H), 2.60-2.70 (m, 2H), 2.55-2.60 (m, 1H), 2.00-2.15 (m, 2H), 1.85-1.95 (m, 2H). ¹⁹F NMR (CD₃OD): δ ppm −67.37.

### Example 120

### 1-(2-hydroxyethyl)-6-((7-(6-(2,2,2-trifluoroethyl)thieno[2,3-d] pyrimidin-4-yl)-2,7-diazaspiro[4.4]nonan-2-yHmethyl)-1H-benzoldlimidazol-2(3H)-one

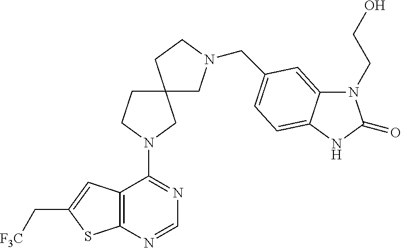 [SMILES: O=c1[nH]c2ccc(CN3CCC4(CCN(c5ncnc6sc(CC(F)(F)F)cc56)C4)C3)cc2n1CCO]

### Step 1. 4-chloro-6-(2,2,2-trifluoroethyl)thieno[2,3-d]pyrimidine

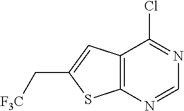 [SMILES: FC(F)(F)Cc1cc2c(Cl)ncnc2s1]

To a solution of 6-(2,2,2-trifluoroethyl)thieno[2,3-d]pyrimidin-4(3H)-one (5 g, 21.35 mmol) in SOCl₂ (85 mL) was added DMF (3.5 mL, anhydrous) under N₂. The reaction mixture was refluxed at 80° C. for 3 h. The mixture was concentrated under reduced pressure. The resulting residue was mixed with EtOAc (500 mL), basified to pH 8-9 with sat. NaHCO₃ (aq). The mixture was separated and the organic layer was washed with brine (2×200 mL), dried over anhydrous Na₂SO₄, filtered, and concentrated under reduced pressure to give the crude product as yellow oil, which was used for the next step without further purification. Yield: 5.2 g (96.4% crude); LCMS method D: Rₜ=1.023 min. (M+H)⁺=253.1, 255.1 (chloro isotopes).

### Step 2. tert-butyl 7-(6-(2,2,2-trifluoroethyl)thieno[2,3-d]pyrimidin-4-yl)-2,7-diazaspiro[4.4]nonane-2-carboxylate

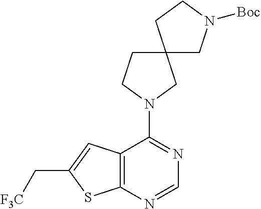 [SMILES: CC(C)(C)OC(=O)N1CCC2(CCN(c3ncnc4sc(CC(F)(F)F)cc34)C2)C1]

To a solution of 4-chloro-6-(2,2,2-trifluoroethyl)thieno[2,3-d]pyrimidine (70 mg, 0.277 mmol) and tert-butyl 2,7-diazaspiro[4.4]nonane-2-carboxylate, semi oxalate salt (75 mg, 0.277 mmol) in isopropanol (ⁱPrOH, 5 mL) was added N,N-diisopropylethylamine (DIEA, 107 mg, 0.831 mmol). The reaction mixture was heated at 95° C. in an oil bath for 18 h. The reaction mixture was then concentrated under reduced pressure to afford the crude product as red solid, which was used for the next step without further purification. Yield: 122.6 mg (100% crude); LCMS method D: Rₜ=1.023 min.; (M+H)⁺=443.2.

### Step 3. 4-(2,7-diazaspiro[4.4]nonan-2-yl)-6-(2,2,2-trifluoroethypthieno[2,3-d]pyrimidine

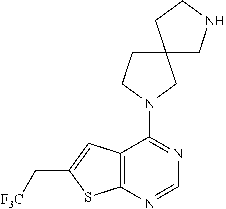 [SMILES: FC(F)(F)Cc1cc2c(N3CCC4(CCNC4)C3)ncnc2s1]

To a solution of tert-butyl 7-(6-(2,2,2-trifluoroethyl)thieno[2,3-d]pyrimidin-4-yl)-2,7-diazaspiro[4.4]nonane-2-carboxylate (122.6 mg, 0.277 mmol) in DCM (3 mL, anhydrous) was added TFA (1 mL) slowly at 0° C. under N₂. The reaction was stirred at 6-22° C. for 30 min. The reaction mixture was concentrated under reduced pressure to afford the crude product as red solid (TFA salt), which was used for the next step without further purification. Yield: crude (100% crude); LCMS method D: Rₜ=0.612 min. (M+H)⁺=343.1.

### Step 4. 1-(2-hydroxyethyl)-6-((7-(6-(2,2,2-trifluoroethyl)thieno[2,3-d]pyrimidin-4-yl)-2,7-diazaspiro[4.4]nonan-2-yl)methyl)-1H-benzo[d]imidazol-2(3H)-one

To a mixture of 4-(2,7-diazaspiro[4.4]nonan-2-yl)-6-(2,2,2-trifluoroethyl)thieno[2,3-d]pyrimidine (50 mg, 0.146 mmol, TFA salt) and 1-(2-hydroxyethyl)-2-oxo-2,3-dihydro-1H-benzo[d]imidazole-5-carbaldehyde (30 mg, 0.146 mmol) was added anhydrous MeOH (5 mL). The reaction mixture was stirred at 65° C. for 1 h under N₂. Then NaBH₃CN (45.8 mg, 0.73 mmol) was added, followed by stirring at 65° C. for 18 h. The reaction mixture was filtered and the filtrate was concentrated under reduced pressure. The residue was diluted with MeOH (5 mL) and the mixture was purified by neutral preparative HPLC method F to afford 1-(2-hydroxyethyl)-6-((7-(6-(2,2,2-trifluoroethyl)thieno[2,3-d]pyrimidin-4-yl)-2,7-diazaspiro[4.4]nonan-2-yl)methyl)-1H-benzo[d]imidazol-2(3H)-one as white solid. Yield: 43.40 mg (55.8% crude); LCMS method D: Rₜ=0.921 min. (M+H)⁺=533.3. ¹H NMR (CD₃OD): δ ppm 8.28 (s, 1H), 7.61 (s, 1H), 7.04-7.19 (m, 3H), 3.95-4.02 (m, 2H), 3.67-3.93 (m, 10H), 2.59-2.85 (m, 4H), 1.89-2.13 (m, 4H). ¹⁹F NMR (CD₃OD): δ ppm −67.71.

### Biological Assays

### Assay 1

Potencies of inhibitor compounds against menin/MLL binding were assessed by AlphaLISA assay using biotinylated (1) wild-type menin or (2) mutated menin (described in Nature (2012) Vol. 482, pp. 542-548) and MLL-AF9 fusion protein bearing a FLAG epitope at its C-terminus. Menin proteins were expressed in E. coli and covalently modified with biotin using EZ-Link™ Sulfo-NHS-Biotin (ThermoFisher Cat. No. 21217) according to manufacturer's protocol. MLL1-1,396 fused to AF91-92 and the C-terminal FLAG peptide was expressed in HEK293 cells and used as a lysate cleared at 21,000×g for 10 min.

Compounds (2 μL of solutions in DMSO) were dispensed in white 96-well half-area plates (Corning Cat. No. 3693) and incubated for 30 min at RT with 5 nM biotinylated menin and appropriate amount of MLL-AF9-FLAG lysate in 40 μL of 50 mM Tris-HCl buffer pH 7.4 containing 5% (v/v) DMSO, 50 mM NaCl, 0.01% (w/v) bovine serum albumin (BSA) and 1 mM DTT. To this incubation mixture, 40 μl of AlphaLISA anti-FLAG acceptor (PerkinElmer Cat. No. AL112C) and streptavidin donor (PerkinElmer Cat. No. 6760002) beads (10 μg/mL each) was added and incubation continued at RT for 60 min. Alpha (amplified luminescent proximity homogeneous assay) signal was measured on an Envision multi-label plate reader at the end of the incubation. All steps were conducted under dim fluorescent light.

Percent inhibition values were calculated based on uninhibited (DMSO) and fully inhibited (10 μM MI-2-2, EMD Millipore Cat. No. 444825) controls. These percent inhibition values were regressed against compound concentrations in the assay using four parameter logit non-linear curve fitting (XLFit, IDBS). The IC₅₀ values were derived from the curve fitting as inflection points on the dose-response curves and are set out in Table 1 below.

### Assay 2: (Cell Assay)

Potencies of inhibitor compounds against menin/MLL were tested in a mammalian cellular two-hybrid assay system. HEK293 cells (American Type Culture Collection (ATCC); Manassas, Va.) were co-transfected with three plasmids: one encoding Gal4-Menin (200 ng per 10⁶ cells), another encoding MLL₁₋₄₆-VP16 (100 ng per 10⁶ cells), and a reporter plasmid pNL3.3-4×UAS (500 ng per 10⁶ cells) encoding secreted NanoLuc® luciferase (Promega) with the upstream 4×UAS repeats for Gal4 binding. Transfection was performed in Opti-MEM® media using Lipofectamine® LTX with Plus™ Reagent (ThermoFisher Scientific) according to manufacturer's protocol. Cells were incubated with transfection reagents at 37° C. 5% CO₂ for 5-6 h, harvested by trypsinization and centrifugation at 300×g for 5 min and resuspended in Gibco® DMEM media (phenol red-free, no antibiotics) containing 10% delipidated fetal bovine serum (FBS) (ThermoFisher Scientific).

Concentration of cells in the media was adjusted to 3.5×10⁵ cells/ml, and 100 μl of the cell suspension was dispensed into each well of 96-well Costar™ black clear bottom cell culture plates (Fisher Scientific). Tested compounds were added as 100 μL of 2× solutions in the media. Final concentration of DMSO was 0.1% in all wells. The cells were incubated with tested compounds at 37° C. 5% CO₂ for 16-18 h.

At the end of the incubation, 40 μL of the conditioned media from each well was transferred into each well of 96-well Costar™ black half-area plates (Fisher Scientific), to which 40 μl of 2× reconstituted NanoGlo® luciferase assay reagent (Promega) was added and thoroughly mixed on a microshaker for 2-3 min. Remaining media in the culture plates was removed by emptying the wells and blotting the plates on filter paper. The cells were lysed with 1× CellTiter-Glo® Luminescent Cell Viability Assay reagent (Promega) for assessing toxicity of the tested compounds. Both, Nano-Glo® and CellTiter-Glo® luminescence intensities were measured on EnVision® multilabel plate reader (PerkinElmer). Percent inhibition values were calculated based on Nano-Glo® luciferase activity observed with the tested compounds vs. DMSO control and commercially available menin inhibitor MI-2-2 (4-(4-(5,5-Dimethyl-4,5-dihydrothiazol-2-yl)piperazin-1-yl)-6-(2,2,2-trifluoroethyl)thieno[2,3-d]pyrimidine) (EMD Millipore, Cat. No. 444825) incubated with cells at 5 μM. These percent inhibition values were regressed against compound concentrations in the assay using non-linear 4-parameter logit curve fitting (XLFit, IDBS). Half maximal inhibitory concentrations (IC₅₀) were calculated as the parameters of the fit corresponding to the inflection points on semi-logarithmic dose-response curves.

Data for Assays 1 and 2 are provided below in Table 1 (“n/a” refers to data not available; “+++” means<100 nM; “++” means≥100 nM and <1000 nM; and “+” means≥1000 nM).

TABLE 1

| | Assay 1 | Assay 1 | |
|---|---|---|---|
| | Wild Type Menin | Mutant Menin | Assay 2 |
| Ex. No. | IC50 (nM) | IC50 (nM) | IC50 (nM) |
| 1 | + | n/a | + |
| 2 | ++ | n/a | + |
| 3 | + | n/a | n/a |
| 4 | + | n/a | n/a |
| 5 | ++ | n/a | n/a |
| 6 | + | n/a | n/a |
| 7 | + | n/a | n/a |
| 8 | + | n/a | n/a |
| 9 | ++ | n/a | n/a |
| 10 | n/a | ++ | n/a |
| 11 | ++ | n/a | n/a |
| 12 | ++ | n/a | n/a |
| 13 | + | n/a | n/a |
| 14 | ++ | n/a | n/a |
| 15 | + | n/a | n/a |
| 16 | + | n/a | n/a |
| 17 | + | n/a | n/a |
| 18 | ++ | n/a | + |
| 19 | ++ | n/a | n/a |
| 20 | ++ | n/a | + |
| 21 | ++ | n/a | n/a |
| 22 | ++ | n/a | + |
| 23 | ++ | n/a | + |
| 24 | ++ | n/a | + |
| 25 | n/a | ++ | + |
| 26 | +++ | n/a | ++ |
| 27 | n/a | ++ | n/a |
| 28 | +++ | n/a | ++ |
| 29 | +++ | n/a | ++ |
| 30 | +++ | n/a | +++ |
| 30a | +++ | n/a | +++ |
| 30b | +++ | n/a | +++ |
| 31 | ++ | n/a | n/a |
| 32 | ++ | n/a | n/a |
| 33 | +++ | n/a | ++ |
| 34 | +++ | n/a | ++ |
| 35 | ++ | n/a | n/a |
| 36 | ++ | n/a | n/a |
| 37 | ++ | n/a | n/a |
| 38 | +++ | n/a | ++ |
| 39 | +++ | n/a | +++ |
| 40 | +++ | n/a | ++ |
| 41 | +++ | n/a | +++ |
| 42 | +++ | n/a | ++ |
| 43 | +++ | n/a | ++ |
| 44 | +++ | n/a | ++ |
| 45 | +++ | n/a | ++ |
| 46 | +++ | n/a | ++ |
| 47 | ++ | n/a | n/a |
| 48 | +++ | n/a | ++ |
| 49 | +++ | n/a | ++ |
| 50 | ++ | n/a | n/a |
| 51 | ++ | n/a | n/a |
| 52 | +++ | n/a | ++ |
| 53 | +++ | n/a | n/a |
| 54 | +++ | n/a | ++ |
| 55 | ++ | n/a | n/a |
| 56 | ++ | n/a | n/a |
| 57 | ++ | n/a | n/a |
| 58 | ++ | n/a | ++ |
| 59 | + | n/a | n/a |
| 60a | + | n/a | n/a |
| 60b | ++ | n/a | n/a |
| 61 | ++ | n/a | + |
| 62 | ++ | n/a | n/a |
| 63 | +++ | n/a | ++ |
| 64 | ++ | n/a | n/a |
| 65 | +++ | n/a | ++ |
| 66 | ++ | n/a | ++ |
| 67 | ++ | n/a | n/a |
| 68 | +++ | n/a | ++ |
| 69 | +++ | n/a | ++ |
| 70 | ++ | n/a | n/a |
| 71 | ++ | n/a | n/a |
| 72 | ++ | n/a | n/a |
| 73 | + | n/a | n/a |
| 74 | + | n/a | n/a |
| 75 | ++ | n/a | ++ |
| 76 | +++ | n/a | ++ |
| 77 | ++ | n/a | n/a |
| 78 | ++ | n/a | n/a |
| 79 | ++ | n/a | n/a |
| 80 | + | n/a | n/a |
| 81 | + | n/a | n/a |
| 82 | ++ | n/a | n/a |
| 83 | +++ | n/a | n/a |
| 84 | +++ | n/a | n/a |
| 85 | + | n/a | n/a |
| 86a | ++ | n/a | n/a |
| 86b | ++ | n/a | n/a |
| 87 | + | n/a | n/a |
| 88 | n/a | + | n/a |
| 89 | + | n/a | n/a |
| 90 | +++ | n/a | ++ |
| 91 | + | n/a | n/a |
| 92 | +++ | n/a | ++ |
| 93 | +++ | n/a | + |
| 94 | ++ | n/a | n/a |
| 95 | + | n/a | n/a |
| 96 | +++ | n/a | +++ |
| 97 | +++ | n/a | ++ |
| 98 | ++ | n/a | ++ |
| 99 | ++ | n/a | + |
| 100 | ++ | n/a | n/a |
| 101 | ++ | n/a | n/a |
| 102 | ++ | n/a | n/a |
| 103 | ++ | n/a | n/a |
| 104 | ++ | n/a | n/a |
| 105 | ++ | n/a | n/a |
| 106 | +++ | n/a | ++ |
| 107 | +++ | n/a | ++ |
| 108 | +++ | n/a | ++ |
| 109 | ++ | n/a | ++ |
| 110 | ++ | n/a | ++ |
| 111 | ++ | n/a | n/a |
| 112 | ++ | n/a | n/a |
| 113 | ++ | n/a | n/a |
| 114 | ++ | n/a | n/a |
| 115 | ++ | n/a | n/a |
| 116 | + | n/a | n/a |
| 117 | + | n/a | n/a |
| 118 | ++ | n/a | n/a |
| 119 | +++ | n/a | ++ |
| 120 | +++ | n/a | + |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound of Formula I: 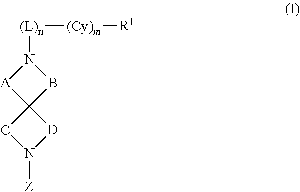
or a pharmaceutically acceptable salt thereof, wherein:
A, B, C, and D are each independently selected from —C(R^{A1})(R^{A2})—, —C(R^{A1})(R^{A2})—C(R^{A1})(R^{A2})—, —C(═O)— and —C(R^{A1})(R^{A2})—C(═O)—, wherein no more than one of A, B, C, and D is —C(R^{A1})(R^{A2})—C(═O)— or —C(═O)—;
L is selected from —C₁₋₆ alkylene- and —(C₁₋₄ alkylene)ₐ-Y—(C₁₋₄ alkylene)_{b}-, wherein the C₁₋₆ alkylene group and any C₁₋₄ alkylene group of the —(C₁₋₄ alkylene)ₐ-Y—(C₁₋₄ alkylene)_{b}- group is optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino;
Y is independently selected from —O—, —S—, —S(═O)—, —S(═O)₂—, —C(═O)—, —C(═O)NR^{y}—, —C(═O)O—, —OC(═O)NR^{y}—, —NR^{y}—, —NR^{y}C(═O)O—, —NR^{y}C(═O)NR^{y}—, —S(═O)₂NR^{y}—, —C(═NR^{z})—, and —C(═NR^{z})—NR^{y}—, wherein each R^{y} is independently selected from H or C₁₋₆ alkyl, and wherein each R^{z} is independently selected from H, C₁₋₆ alkyl, and CN;
Cy is C₆₋₁₄ aryl, C₃₋₁₈ cycloalkyl, 5-16 membered heteroaryl, or 4-18 membered heterocycloalkyl, each of which is optionally substituted with 1, 2, 3, or 4 substituents independently selected from R^{Cy};
each R^{Cy} is independently selected from halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR¹R^{d1};
R¹ is H, Cy¹, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2} and S(O)₂NR^{c2}R^{d2}, wherein said C₁₋₆ alkyl, C₂₋₆ alkenyl, and C₂₋₆ alkynyl are each optionally substituted by 1, 2, 3, or 4 substituents independently selected from CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
Z is a group of Formula (Z-1) or (Z-2): 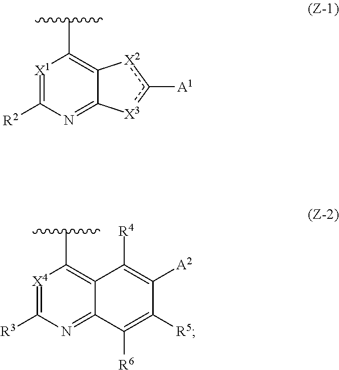
X¹ is selected from CR⁷ and N;
X² is selected from S, CR⁸, and N;
X³ is selected from CH, S, O, and NR^{N};
X⁴ is selected from CR⁹ and N; [symbol]
represents a single or double bond, wherein one [symbol] in the group of Formula (Z-1) is a double bond and the other is a single bond;
each R², R³, R⁴, R⁵, R⁶, R⁷, R⁸, and R⁹ is independently selected from H, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, and C₂₋₆ alkynyl are each optionally substituted by 1, 2, 3, or 4 substituents independently selected from CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
R^{N} is H or C₁₋₆ alkyl optionally substituted by 1, 2, 3, or 4 substituents independently selected from CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
A¹ is selected from H, halo, C₁₋₆ alkyl, C₁₋₄ haloalkyl, C₁₋₄ haloalkoxy, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, are each optionally substituted by 1, 2, 3, or 4 substituents independently selected from CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
A² is selected from H, halo, C₁₋₆ alkyl, C₁₋₄ haloalkyl, C₁₋₄ haloalkoxy, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{A1} is independently selected form H, halo, C₁₋₄ alkyl, C₁₋₄ alkoxy, C₁₋₄ haloalkyl, C₁₋₄ haloalkoxy, CN, NO₂, and OH;
each R^{A2} is independently selected form H, halo, C₁₋₄ alkyl, C₁₋₄ alkoxy, C₁₋₄ haloalkyl, C₁₋₄ haloalkoxy, CN, NO₂, and OH;
each Cy¹ is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy1};
each R^{Cy1} is independently selected from halo, C₁₋₆ alkyl, C₁₋₄ haloalkyl, C₁₋₄ cyanoalkyl, C₂₋₆ alkenyl, C₂₋₆ alkynyl, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, C(═NR^{e6})NR^{c6}R^{d6}, NR^{c6}C(═NR^{e6})NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}, wherein said C₁₋₆ alkyl, C₂₋₆ alkenyl, and C₂₋₆ alkynyl are each optionally substituted by 1, 2, 3, or 4 substituents independently selected from CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, C(═NR^{e6})NR^{c6}R^{d6}, NR^{c6}C(═NR^{e6})NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6};
each R^{a1}, R^{b1}, R^{c1}, R^{d1}, R^{a2}, R^{b2}, R^{c2}, R^{d2}, R^{a3}, R^{b3}, R^{c3}, R^{d3}, R^{a4}, R^{b4}, R^{c4}, R^{d4}, R^{a5}, R^{b5}, R^{c5}, R^{d5}, R^{a6}, R^{b6}, R^{c6}, and R^{d6} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycloalkyl)-C₁₋₆ alkyl, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl, C₃₋₁₀ cycloalky-C₁₋₆ alkyl, (5-10 membered heteroaryl)-C₁₋₆ alkyl, and (4-10 membered heterocycloalkyl)-C₁₋₆ alkyl are each optionally substituted with 1, 2, 3, 4, or 5 substituents independently selected from R^{g};
each R^{e1}, R^{e2}, R^{e3}, R^{e4}, R^{e5}, and R^{e6} is independently selected from H, C₁₋₄ alkyl, and CN;
each R^{g} is independently selected from the group consisting of OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkyl sulfonyl, carboxy, C₁₋₆ alkylcarbonyl, and C₁₋₆ alkoxycarbonyl;
n is 0 or 1;
m is 0 or 1;
a is 0 or 1; and
b is 0 or 1,
wherein any cycloalkyl or heterocycloalkyl group is optionally further substituted by 1 or 2 oxo groups; and
wherein the compound is not:

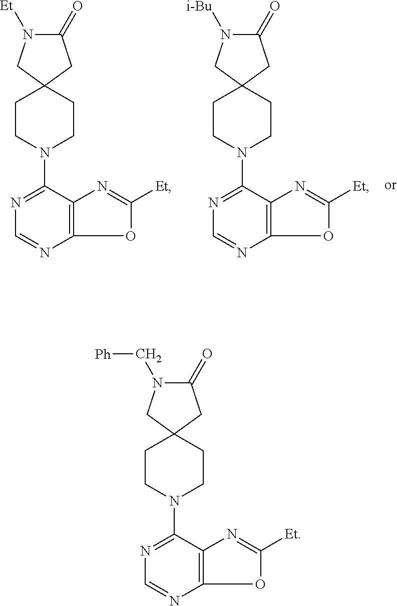

2. The compound of claim 1, or a pharmaceutically acceptable salt thereof, wherein the moiety formed by A, B, C, D, the nitrogen atom to which A and B are attached, the nitrogen atom to which C and D are attached, and the spirocyclic carbon atom, is selected from the following spirocycles having Formulae (i) to (x): 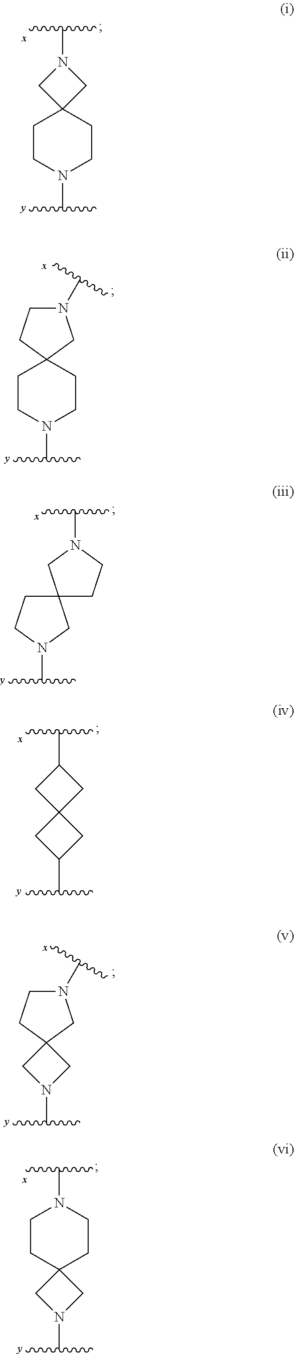 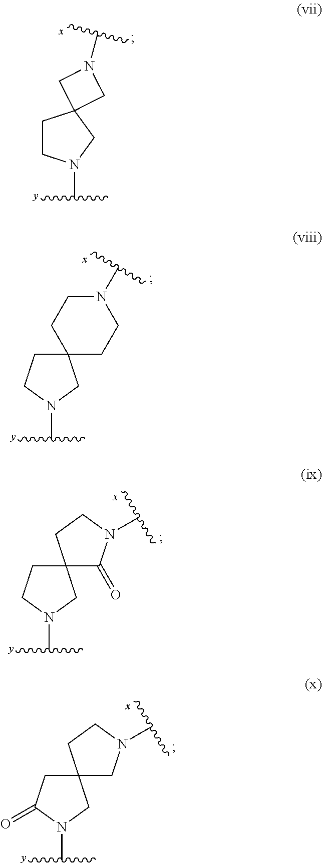 [SMILES: *N1CCC2(C1)CN(*)C2 ; *N1CCC2(CC1)CCN(*)C2 ; *N1CCC2(CCN(*)C2=O)C1 ; *N1CCC2(CC(=O)N(*)C2)C1]
wherein “x” indicates the point of attachment to L and “y” indicates the point of attachment to Z.

3. (canceled)

4. The compound of claim 1, or a pharmaceutically acceptable salt thereof, wherein L is selected from methylene, ethylene, and —(C═O)—(C₁₋₄ alkylene)_{b}-, wherein the methylene, ethylene, and C₁₋₄ alkylene group are each optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino.

5.-16. (canceled)

17. The compound of claim 1, or a pharmaceutically acceptable salt thereof, wherein Z is a group of Formula (Z-1):

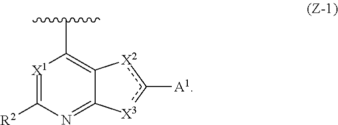

18.-26. (canceled)

27. The compound of claim 1, or a pharmaceutically acceptable salt thereof, wherein A¹ is C₁₋₄ haloalkyl.

28. The compound of claim 1, or a pharmaceutically acceptable salt thereof, wherein A¹ is 2,2,2-trifluoroethyl.

29. The compound of claim 1, or a pharmaceutically acceptable salt thereof, wherein Z is a group of Formula (Z-2):

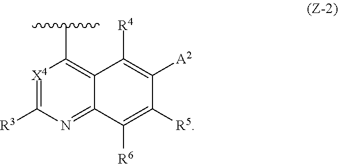

30.-38. (canceled)

39. The compound of claim 1, or a pharmaceutically acceptable salt thereof, having Formula (IIa), (IIb), (IIc), (IId), (IIe), (IIf), (IIg), (IIg), (IIi), or (IIj):

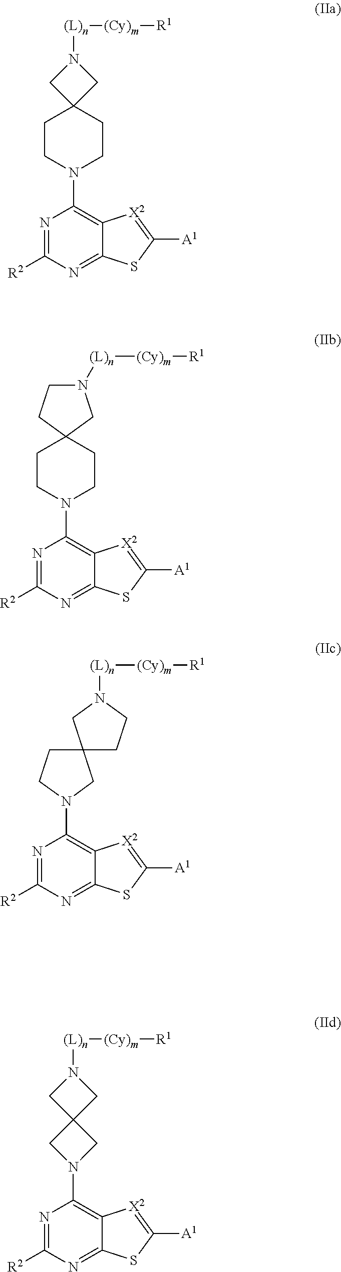

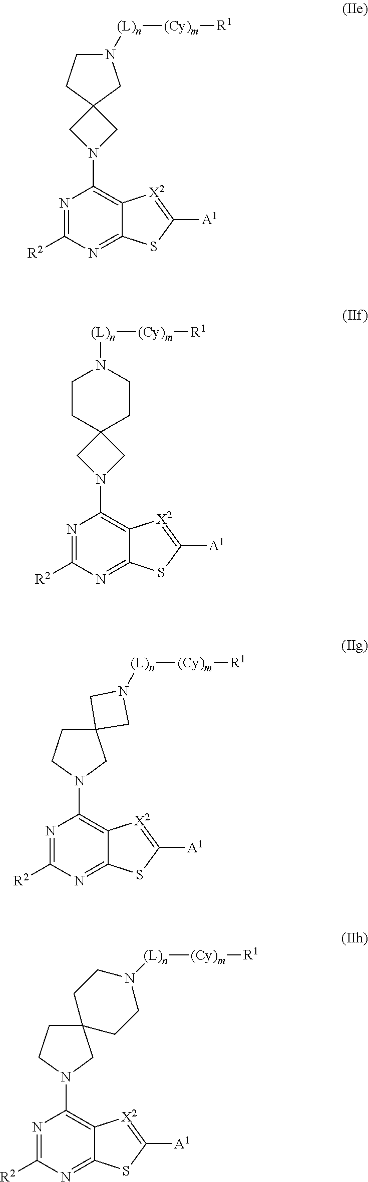

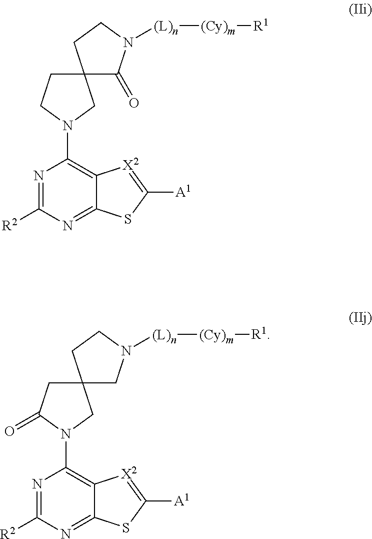

40. The compound of claim 1, or a pharmaceutically acceptable salt thereof, having Formula (IIIa), (IIIb), or (IIIc):

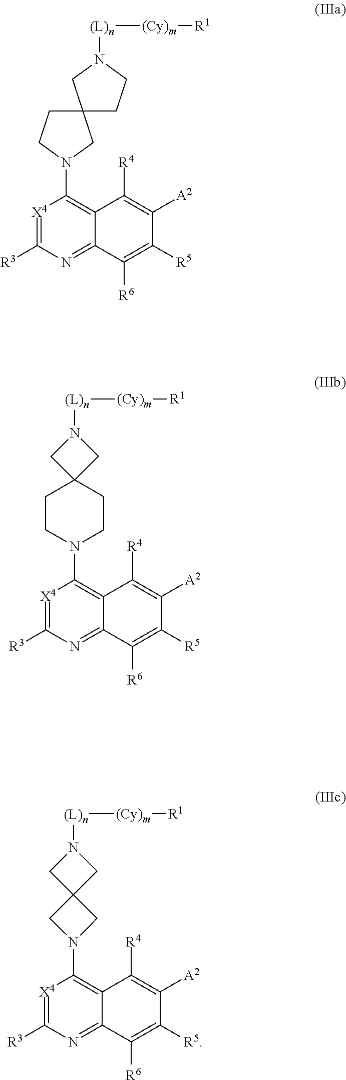

41. The compound of claim 1 selected from:
4-(2-benzyl-2,7-diazaspiro[3.5]nonan-7-yl)-6-(2,2,2-trifluoroethyl) thieno[2,3-d]pyrimidine;
4-(2-isobutyl-2,7-diazaspiro[3.5]nonan-7-yl)-6-(2,2,2-trifluoroethyl)thieno[2,3-d]pyrimidine;
4-(2-isopropyl-2,7-diazaspiro[3.5]nonan-7-yl)-6-(2,2,2-trifluoroethyl)thieno[2,3-d]pyrimidine;
4-(2-((tetrahydrofuran-2-yl)methyl)-2,7-diazaspiro[3.5]nonan-7-yl)-6-(2,2,2-trifluoroethyl)thieno[2,3-d]pyrimidine;
4-(2-((tetrahydrofuran-3-yl)methyl)-2,7-diazaspiro[3.5]nonan-7-yl)-6-(2,2,2-trifluoroethyl)thieno[2,3-d]pyrimidine;
4-(2-((tetrahydro-2H-pyran-2-yl)methyl)-2,7-diazaspiro[3.5] nonan-7-yl)-6-(2,2,2-trifluoroethyl)thieno[2,3-d]pyrimidine;
4-(2-((tetrahydro-2H-pyran-3-yl)methyl)-2,7-diazaspiro[3.5] nonan-7-yl)-6-(2,2,2-trifluoroethyl)thieno[2,3-d]pyrimidine;
4-(2-(tetrahydro-2H-pyran-4-yl)-2,7-diazaspiro[3.5]nonan-7-yl)-6-(2,2,2-trifluoroethyl)thieno[2,3-d]pyrimidine;
4-(2-cyclohexyl-2,7-diazaspiro[3.5]nonan-7-yl)-6-(2,2,2-trifluoroethyl)thieno[2,3-d]pyrimidine;
4-(2-(5,5-dimethyl-4,5-dihydrothiazol-2-yl)-2,7-diazaspiro[3.5] nonan-7-yl)-6-(2,2,2-trifluoroethyl)thieno[2,3-d]pyrimidine;
4-(2-((3,3-difluorocyclohexyl)methyl)-2,7-diazaspiro[3.5]nonan-7-yl)-6-(2,2,2-trifluoroethyl)thieno[2,3-d]pyrimidine;
4-(2-((4,4-difluorocyclohexyl)methyl)-2,7-diazaspiro[3.5] nonan-7-yl)-6-(2,2,2-trifluoroethyl)thieno[2,3-d]pyrimidine;
4-(2-((3-methoxycyclobutyl)methyl)-2,7-diazaspiro[3.5]nonan-7-yl)-6-(2,2,2-trifluoroethyl)thieno[2,3-d]pyrimidine;
4-(2-((3,3-difluorocyclobutyl)methyl)-2,7-diazaspiro[3.5]nonan-7-yl)-6-(2,2,2-trifluoroethyl)thieno[2,3-d]pyrimidine;
2-methyl-1-(7-(6-(2,2,2-trifluoroethyl)thieno[2,3-d]pyrimidin-4-yl)-2,7-diazaspiro[3.5]nonan-2-yl)propan-2-ol;
4-(2-(2-fluoro-2-methylpropyl)-2,7-diazaspiro[3.5]nonan-7-yl)-6-(2,2,2-trifluoroethyl)thieno[2,3-d]pyrimidine;
3-amino-3-methyl-1-(7-(6-(2,2,2-trifluoroethyl)thieno[2,3-d]pyrimidin-4-yl)-2,7-diazaspiro[3.5]nonan-2-yl)butan-1-one;
4-(2-neopentyl-2,7-diazaspiro[3.5]nonan-7-yl)-6-(2,2,2-trifluoroethyl)thieno[2,3-d]pyrimidine;
4-(2-((tetrahydro-2H-pyran-4-yl)methyl)-2,7-diazaspiro[3.5]nonan-7-yl)-6-(2,2,2-trifluoroethyl)thieno[2,3-d]pyrimidine;
3-((7-(6-(2,2,2-trifluoroethyl)thieno[2,3-d]pyrimidin-4-yl)-2,7-diazaspiro[3.5]nonan-2-yl)methyl)-1H-indole-6-carbonitrile;
4-methyl-5-((7-(6-(2,2,2-trifluoroethyl)thieno[2,3-d]pyrimidin-4-yl)-2,7-diazaspiro[3.5]nonan-2-yl)methyl)-1H-indole-2-carbonitrile;
4-(2-(2-methylbutyl)-2,7-diazaspiro[3.5]nonan-7-yl)-6-(2,2,2-trifluoroethyl)thieno[2,3-d]pyrimidine;
4-(2-benzyl-2,8-diazaspiro[4.5]decan-8-yl)-6-(2,2,2-trifluoroethyl) thieno[2,3-d]pyrimidine;
4-(2-isobutyl-2,8-diazaspiro[4.5]decan-8-yl)-6-(2,2,2-trifluoroethyl)thieno[2,3-d]pyrimidine;
4-(2-isopropyl-2,8-diazaspiro[4.5]decan-8-yl)-6-(2,2,2-trifluoroethyl)thieno[2,3-d]pyrimidine;
4-methyl-5-((8-(6-(2,2,2-trifluoroethyl)thieno[2,3-d]pyrimidin-4-yl)-2,8-diazaspiro[4.5]decan-2-yl)methyl)-1H-indole-2-carbonitrile;
4-(2-(5,5-dimethyl-4,5-dihydrothiazol-2-yl)-2,8-diazaspiro[4.5]decan-8-yl)-6-(2,2,2-trifluoroethyl)thieno[2,3-d]pyrimidine;
4-(7-((1H-indol-5-yl)methyl)-2,7-diazaspiro[4.4]nonan-2-yl)-6-(2,2,2-trifluoroethyl)thieno[2,3-d]pyrimidine;
4-(7-((1H-indol-6-yl)methyl)-2,7-diazaspiro[4.4]nonan-2-yl)-6-(2,2,2-trifluoroethyl)thieno[2,3-d]pyrimidine;
4-methyl-5-((7-(6-(2,2,2-trifluoroethyl)thieno[2,3-d]pyrimidin-4-yl)-2,7-diazaspiro[4.4]nonan-2-yl)methyl)-1H-indole-2-carbonitrile;
(R)-4-methyl-5-((7-(6-(2,2,2-trifluoroethyl)thieno[2,3-d]pyrimidin-4-yl)-2,7-diazaspiro[4.4]nonan-2-yl)methyl)-1H-indole-2-carbonitrile;
(S)-4-methyl-5-((7-(6-(2,2,2-trifluoroethyl)thieno[2,3-d]pyrimidin-4-yl)-2,7-diazaspiro[4.4]nonan-2-yl)methyl)-1H-indole-2-carbonitrile;
4-(7-((tetrahydrofuran-3-yl)methyl)-2,7-diazaspiro[4.4]nonan-2-yl)-6-(2,2,2-trifluoroethyl)thieno[2,3-d]pyrimidine;
4-(7-((4,4-difluorocyclohexyl)methyl)-2,7-diazaspiro[4.4]nonan-2-yl)-6-(2,2,2-trifluoroethyl)thieno[2,3-d]pyrimidine;
4-((7-(6-(2,2,2-trifluoroethyl)thieno[2,3-d]pyrimidin-4-yl)-2,7-diazaspiro[4.4]nonan-2-yl)methyl)benzonitrile;
N-(4-((7-(6-(2,2,2-trifluoroethyl)thieno[2,3-d]pyrimidin-4-yl)-2,7-diazaspiro[4.4]nonan-2-yl)methyl)phenyl)acetamide;
3-((7-(6-(2,2,2-Trifluoroethyl)thieno[2,3-d]pyrimidin-4-yl)-2,7-diazaspiro[4.4]nonan-2-yl)methyl)benzonitrile;
4-(7-(2-chloro-4-(methylsulfonyl)benzyl)-2,7-diazaspiro[4.4]nonan-2-yl)-6-(2,2,2-trifluoroethyl)thieno[2,3-d]pyrimidine;
6-(2,2,2-trifluoroethyl)-4-(7-(((1r,4r)-4-(trifluoromethyl)cyclohexyl)methyl)-2,7-diazaspiro[4.4]nonan-2-yl)thieno[2,3-d]pyrimidine;
N-(4-((7-(6-(2,2,2-trifluoroethyl)thieno[2,3-d]pyrimidin-4-yl)-2,7-diazaspiro[4.4]nonan-2-yl)methyl)phenyl)methanesulfonamide;
5-((7-(6-(2,2,2-trifluoroethyl)thieno[2,3-d]pyrimidin-4-yl)-2,7-diazaspiro[4.4]nonan-2-yl)methyl)-1H-benzo[d]imidazol-2(3H)-one;
6-((7-(6-(2,2,2-trifluoroethyl)thieno[2,3-d]pyrimidin-4-yl)-2,7-diazaspiro[4.4]nonan-2-yl)methyl)benzo[d]oxazol-2(3H)-one;
1-methyl-5-((7-(6-(2,2,2-trifluoroethyl)thieno[2,3-d]pyrimidin-4-yl)-2,7-diazaspiro[4.4]nonan-2-yl)methyl)-1,3-dihydro-2H-benzo[d]imidazol-2-one;
N-(3-methyl-4-((7-(6-(2,2,2-trifluoroethyl)thieno[2,3-d]pyrimidin-4-yl)-2,7-diazaspiro[4.4]nonan-2-yl)methyl)phenyl)acetamide;
4-(7-benzyl-2,7-diazaspiro[4.4]nonan-2-yl)-6-(2,2,2-trifluoroethyl)thieno[2,3-d]pyrimidine;
N-(3-chloro-4-((7-(6-(2,2,2-trifluoroethyl)thieno[2,3-d]pyrimidin-4-yl)-2,7-diazaspiro[4.4]nonan-2-yl)methyl)phenyl)acetamide;
N-(3-fluoro-4-((7-(6-(2,2,2-trifluoroethyl)thieno[2,3-d]pyrimidin-4-yl)-2,7-diazaspiro[4.4]nonan-2-yl)methyl)phenyl)acetamide;
Methyl ((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)carbamate;
N-((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)acetamide;
N-((1r,4r)-4-((7-(6-(2,2,2-trifluoroethyl)thieno[2,3-d]pyrimidin-4-yl)-2,7-diazaspiro[4.4]nonan-2-yl)methyl)cyclohexyl)methanesulfonamide;
N-((1s,4s)-4-((7-(6-(2,2,2-trifluoroethyl)thieno[2,3-d]pyrimidin-4-yl)-2,7-diazaspiro[4.4]nonan-2-yl)methyl)cyclohexyl)methanesulfonamide;
N-((1s,4s)-4-((7-(6-(2,2,2-trifluoroethyl)thieno[2,3-d]pyrimidin-4-yl)-2,7-diazaspiro[4.4]nonan-2-yl)methyl)cyclohexyl)acetamide;
methyl ((1s,4s)-4-((7-(6-(2,2,2-trifluoroethyl)thieno[2,3-d]pyrimidin-4-yl)-2,7-diazaspiro[4.4]nonan-2-yl)methyl)cyclohexyl)carbamate;
N-(3-((7-(6-(2,2,2-trifluoroethyl)thieno[2,3-d]pyrimidin-4-yl)-2,7-diazaspiro[4.4]nonan-2-yl)methyl)phenyl)acetamide;
methyl (3-((7-(6-(2,2,2-trifluoroethyl)thieno[2,3-d]pyrimidin-4-yl)-2,7-diazaspiro[4.4]nonan-2-yl)methyl)phenyl)carbamate;
N-(3-methyl-4-((7-(6-(2,2,2-trifluoroethyl)thieno[2,3-d]pyrimidin-4-yl)-2,7-diazaspiro[4.4]nonan-2-yl)methyl)phenyl)methanesulfonamide;
piperidin-3-yl(7-(6-(2,2,2-trifluoroethyl)thieno[2,3-d]pyrimidin-4-yl)-2,7-diazaspiro[4.4]nonan-2-yl)methanone;
piperidin-2-yl(7-(6-(2,2,2-trifluoroethyl)thieno[2,3-d]pyrimidin-4-yl)-2,7-diazaspiro[4.4]nonan-2-yl)methanone;
4-(7-prolyl-2,7-diazaspiro[4.4]nonan-2-yl)-6-(2,2,2-trifluoroethyl)thieno[2,3-d]pyrimidine;
2-amino-2-methyl-1-(7-(6-(2,2,2-trifluoroethyl)thieno[2,3-d]pyrimidin-4-yl)-2,7-diazaspiro[4.4]nonan-2-yl)propan-1-one;
2-benzyl-7-(6-(2,2,2-trifluoroethyl)thieno[2,3-d]pyrimidin-4-yl)-2,7-diazaspiro[4.4]nonan-1-one;
((1s,4s)-4-methoxycyclohexyl)(6-(6-(2,2,2-trifluoroethyl)thieno[2,3-d]pyrimidin-4-yl)-2,6-diazaspiro[3.3]heptan-2-yl)methanone;
((1r,4r)-4-methoxycyclohexyl)(6-(6-(2,2,2-trifluoroethyl)thieno[2,3-d]pyrimidin-4-yl)-2,6-diazaspiro[3.3]heptan-2-yl)methanone;
2-(4-((methylsulfonyl)methyl)phenyl)-1-(6-(6-(2,2,2-trifluoroethyl)thieno[2,3-d]pyrimidin-4-yl)-2,6-diazaspiro[3.3]heptan-2-yl)ethan-1-one;
2-(4-(methylsulfonyl)phenyl)-1-(6-(6-(2,2,2-trifluoroethyl)thieno[2,3-d]pyrimidin-4-yl)-2,6-diazaspiro[3.3]heptan-2-yl)ethan-1-one;
4-(6-((3,3-difluorocyclohexyl)methyl)-2,6-diazaspiro[3.3]heptan-2-yl)-6-(2,2,2-trifluoroethyl)thieno[2,3-d]pyrimidine;
4-(6-((4,4-dimethylcyclohexyl)methyl)-2,6-diazaspiro[3.3]heptan-2-yl)-6-(2,2,2-trifluoroethyl)thieno[2,3-d]pyrimidine;
4-(6-((3,3-dimethylcyclohexyl)methyl)-2,6-diazaspiro[3.3]heptan-2-yl)-6-(2,2,2-trifluoroethyl)thieno[2,3-d]pyrimidine;
4-(6-((tetrahydro-2H-pyran-4-yl)methyl)-2,6-diazaspiro[3.3]heptan-2-yl)-6-(2,2,2-trifluoroethyl)thieno[2,3-d]pyrimidine;
4-methyl-5-((6-(6-(2,2,2-trifluoroethyl)thieno[2,3-d]pyrimidin-4-yl)-2,6-diazaspiro[3.3]heptan-2-yl)methyl)-1H-indole-2-carbonitrile;
4-(6-(cyclopentylmethyl)-2,6-diazaspiro[3.3]heptan-2-yl)-6-(2,2,2-trifluoroethyl)thieno[2,3-d]pyrimidine;
4-(6-(cyclohexylmethyl)-2,6-diazaspiro[3.3]heptan-2-yl)-6-(2,2,2-trifluoroethyl)thieno[2,3-d]pyrimidine;
4-(6-((4,4-difluorocyclohexyl)methyl)-2,6-diazaspiro[3.3]heptan-2-yl)-6-(2,2,2-trifluoroethyl)thieno[2,3-d]pyrimidine;
N-((1r,4r)-4-((6-(6-(2,2,2-trifluoroethyl)thieno[2,3-d]pyrimidin-4-yl)-2,6-diazaspiro[3.3]heptan-2-yl)methyl)cyclohexyl)ethanesulfonamide;
N-((1r,4r)-4-((6-(6-(2,2,2-trifluoroethyl)thieno[2,3-d]pyrimidin-4-yl)-2,6-diazaspiro[3.3]heptan-2-yl)methyl)cyclohexyl)cyclopropanesulfonamide;
4-(6-(oxetan-3-ylmethyl)-2,6-diazaspiro[3.3]heptan-2-yl)-6-(2,2,2-trifluoroethyl)thieno[2,3-d]pyrimidine;
4-(6-((3,3-difluorocyclobutyl)methyl)-2,6-diazaspiro[3.3]heptan-2-yl)-6-(2,2,2-trifluoroethyl)thieno[2,3-d]pyrimidine;
4-(6-((4-methoxycyclohexyl)methyl)-2,6-diazaspiro[3.3]heptan-2-yl)-6-(2,2,2-trifluoroethyl)thieno[2,3-d]pyrimidine;
4-((6-(6-(2,2,2-trifluoroethyl)thieno[2,3-d]pyrimidin-4-yl)-2,6-diazaspiro[3.3]heptan-2-yl)methyl)cyclohexan-1-ol;
3-amino-3-methyl-1-(6-(6-(2,2,2-trifluoroethyl)thieno[2,3-d]pyrimidin-4-yl)-2,6-diazaspiro[3.3]heptan-2-yl)butan-1-one;
piperidin-3-yl(6-(6-(2,2,2-trifluoroethyl)thieno[2,3-d]pyrimidin-4-yl)-2,6-diazaspiro[3.3]heptan-2-yl)methanone;
piperidin-2-yl(6-(6-(2,2,2-trifluoroethyl)thieno[2,3-d]pyrimidin-4-yl)-2,6-diazaspiro[3.3]heptan-2-yl)methanone;
7-(2-isopropyl-2,7-diazaspiro[3.5]nonan-7-yl)-2-(2,2,2-trifluoroethyl)thiazolo[5,4-d]pyrimidine;
7-(2-isobutyl-2,7-diazaspiro[3.5]nonan-7-yl)-2-(2,2,2-trifluoroethyl) thiazolo[5,4-d]pyrimidine;
7-(7-benzyl-2,7-diazaspiro[4.4]nonan-2-yl)-2-(2,2,2-trifluoroethyl)thiazolo[5,4-d]pyrimidine;
4-methyl-5-((7-(2-(2,2,2-trifluoroethyl)thiazolo[5,4-d]pyrimidin-7-yl)-2,7-diazaspiro[4.4]nonan-2-yl)methyl)-1H-indole-2-carbonitrile;
N-(4-((7-(2-(2,2,2-trifluoroethyl)thiazolo[5,4-d]pyrimidin-7-yl)-2,7-diazaspiro[4.4]nonan-2-yl)methyl)phenyl)methanesulfonamide;
4-(2-isobutyl-2,7-diazaspiro[3.5]nonan-7-yl)-6-(2,2,2-trifluoroethyl) quinoline;
(S)-4-methyl-5-((7-(6-(2,2,2-trifluoroethyl)quinolin-4-yl)-2,7-diazaspiro[4.4]nonan-2-yl)methyl)-1H-indole-2-carbonitrile;
(R)-4-methyl-5-((7-(6-(2,2,2-trifluoroethyl)quinolin-4-yl)-2,7-diazaspiro[4.4]nonan-2-yl)methyl)-1H-indole-2-carbonitrile;
4-(2-(cyclopropylmethyl)-2,7-diazaspiro[3.5]nonan-7-yl)-6-(2,2,2-trifluoroethyl)quinoline;
5,5-dimethyl-2-(6-(6-(trifluoromethoxy)quinazolin-4-yl)-2,6-diazaspiro[3.3]heptan-2-yl)-4,5-dihydrothiazole;
4-(2-isobutyl-2,7-diazaspiro[3.5]nonan-7-yl)-6-(2,2,2-trifluoroethyl)quinazoline;
4-methyl-5-((7-(6-(2,2,2-trifluoroethyl)quinazolin-4-yl)-2,7-diazaspiro[4.4]nonan-2-yl)methyl)-1H-indole-2-carbonitrile;
4-(6-((tetrahydro-2H-pyran-4-yl)methyl)-2,6-diazaspiro[3.3]heptan-2-yl)-6-(2,2,2-trifluoroethyl)quinazoline;
4-methyl-5-((2-(6-(2,2,2-trifluoroethyl)thieno[2,3-d]pyrimidin-4-yl)-2,6-diazaspiro[3.4]octan-6-yl)methyl)-1H-indole-2-carbonitrile;
4-(6-((tetrahydro-2H-pyran-4-yl)methyl)-2,6-diazaspiro[3.4]octan-2-yl)-6-(2,2,2-trifluoroethyl)thieno[2,3-d]pyrimidine;
4-(6-(2-methylbutyl)-2,6-diazaspiro[3.4]octan-2-yl)-6-(2,2,2-trifluoroethyl)thieno[2,3-d]pyrimidine;
4-(6-benzyl-2,6-diazaspiro[3.4]octan-2-yl)-6-(2,2,2-trifluoroethyl)thieno[2,3-d]pyrimidine;
4-methyl-5-((2-(6-(2,2,2-trifluoroethyl)thieno[2,3-d]pyrimidin-4-yl)-2,7-diazaspiro[3.5]nonan-7-yl)methyl)-1H-indole-2-carbonitrile;
N-(4-((2-(6-(2,2,2-trifluoroethyl)thieno[2,3-d]pyrimidin-4-yl)-2,7-diazaspiro[3.5]nonan-7-yl)methyl)phenyl)methanesulfonamide;
4-(7-benzyl-2,7-diazaspiro[3.5]nonan-2-yl)-6-(2,2,2-trifluoroethyl)thieno[2,3-d]pyrimidine;
4-(7-((tetrahydro-2H-pyran-4-yl)methyl)-2,7-diazaspiro[3.5]nonan-2-yl)-6-(2,2,2-trifluoroethyl)thieno[2,3-d]pyrimidine;
4-(7-(2-methylbutyl)-2,7-diazaspiro[3.5]nonan-2-yl)-6-(2,2,2-trifluoroethyl)thieno[2,3-d]pyrimidine;
4-(7-isobutyl-2,7-diazaspiro[3.5]nonan-2-yl)-6-(2,2,2-trifluoroethyl)thieno[2,3-d]pyrimidine;
6-(2,2,2-trifluoroethyl)-4-(7-(((1r,40-4-(trifluoromethyl)cyclohexyl)methyl)-2,7-diazaspiro[3.5]nonan-2-yl)thieno[2,3-d]pyrimidine;
N-(4-((2-(6-(2,2,2-trifluoroethyl)thieno[2,3-d]pyrimidin-4-yl)-2,7-diazaspiro[3.5]nonan-7-yl)methyl)phenyl)acetamide;
4-(2-(6-(2,2,2-trifluoroethyl)thieno[2,3-d]pyrimidin-4-yl)-2,7-diazaspiro[3.5]nonan-7-yl)tetrahydro-2H-thiopyran 1,1-dioxide;
N-((1r,4r)-4-((2-(6-(2,2,2-trifluoroethyl)thieno[2,3-d]pyrimidin-4-yl)-2,7-diazaspiro[3.5]nonan-7-yl)methyl)cyclohexyl)acetamide;
N-((1r,4r)-4-((2-(6-(2,2,2-trifluoroethyl)thieno[2,3-d]pyrimidin-4-yl)-2,7-diazaspiro[3.5]nonan-7-yl)methyl)cyclohexyl)methanesulfonamide;
methyl ((1r,4r)-4-((2-(6-(2,2,2-trifluoroethyl)thieno[2,3-d]pyrimidin-4-yl)-2,7-diazaspiro[3.5]nonan-7-yl)methyl)cyclohexyl)carbamate;
4-(2-isobutyl-2,6-diazaspiro[3.4]octan-6-yl)-6-(2,2,2-trifluoroethyl)thieno[2,3-d]pyrimidine;
4-(2-((tetrahydro-2H-pyran-4-yl)methyl)-2,6-diazaspiro[3.4]octan-6-yl)-6-(2,2,2-trifluoroethyl)thieno[2,3-d]pyrimidine;
4-(2-(2-methylbutyl)-2,6-diazaspiro[3.4]octan-6-yl)-6-(2,2,2-trifluoroethyl)thieno[2,3-d]pyrimidine;
4-(2-benzyl-2,6-diazaspiro[3.4]octan-6-yl)-6-(2,2,2-trifluoroethyl)thieno[2,3-d]pyrimidine;
4-methyl-5-((6-(6-(2,2,2-trifluoroethyl)thieno[2,3-d]pyrimidin-4-yl)-2,6-diazaspiro[3.4]octan-2-yl)methyl)-1H-indole-2-carbonitrile;
4-(8-isobutyl-2,8-diazaspiro[4.5]decan-2-yl)-6-(2,2,2-trifluoroethyl)thieno[2,3-d]pyrimidine;
4-(8-(2-methylbutyl)-2,8-diazaspiro[4.5]decan-2-yl)-6-(2,2,2-trifluoroethyl)thieno[2,3-d]pyrimidine;
4-(8-((tetrahydro-2H-pyran-4-yl)methyl)-2,8-diazaspiro[4.5]decan-2-yl)-6-(2,2,2-trifluoroethyl)thieno[2,3-d]pyrimidine;
4-(8-benzyl-2,8-diazaspiro[4.5]decan-2-yl)-6-(2,2,2-trifluoroethyl)thieno[2,3-d]pyrimidine;
4-methyl-5-((2-(6-(2,2,2-trifluoroethyl)thieno[2,3-d]pyrimidin-4-yl)-2,8-diazaspiro[4.5]decan-8-yl)methyl)-1H-indole-2-carbonitrile;
7-((2-oxo-2,3-dihydro-1H-benzo[d]imidazol-5-yl)methyl)-2-(6-(2,2,2-trifluoroethyl)thieno[2,3-d]pyrimidin-4-yl)-2,7-diazaspiro[4.4]nonan-3-one;
1-(2-hydroxyethyl)-5-((7-(6-(2,2,2-trifluoroethyl)thieno[3,2-d]pyrimidin-4-yl)-2,7-diazaspiro[4.4]nonan-2-yl)methyl)-1H-benzo[d]imidazol-2(3H)-one; and
1-(2-hydroxyethyl)-6-((7-(6-(2,2,2-trifluoroethyl)thieno[2,3-d] pyrimidin-4-yl)-2,7-diazaspiro[4.4]nonan-2-yl)methyl)-1H-benzo[d]imidazol-2(3H)-one;
or a pharmaceutically acceptable salt thereof.

42. A pharmaceutical composition comprising a compound of claim 1, or a pharmaceutically acceptable salt thereof, and at least one pharmaceutically acceptable carrier.

43. A method of inhibiting the interaction between menin and MLL comprising contacting the menin and MLL with a compound of claim 1, or a pharmaceutically acceptable salt thereof.

44. A method of treating cancer in a patient comprising administering to the patient a therapeutically effective amount of a compound of claim 1, or a pharmaceutically acceptable salt thereof.

45. The method of claim 44 wherein the cancer is a hematological cancer.

46. The method of claim 45 wherein the cancer is leukemia.

47. The method of claim 45 wherein the cancer is lymphoma.

48. The method of claim 45 wherein the cancer is mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Szary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.

49. A method of treating insulin resistance, pre-diabetes, diabetes, or risk of diabetes in a patient comprising administering to the patient a therapeutically effective amount of a compound of claim 1, or a pharmaceutically acceptable salt thereof.

50. A method of treating hyperglycemia in a patient comprising administering to the patient a therapeutically effective amount of a compound of claim 1, or a pharmaceutically acceptable salt thereof.
